SEQUENCE LISTING (56 sequences)

SEQ ID NO: 1 - protein, 215 aa, Mus musculus [sequence omitted]

SEQ ID NO: 2 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 3 - protein, 6 aa, synthetic construct
GPSLKL

SEQ ID NO: 4 - protein, 10 aa, synthetic construct
PPKGETKKKF

SEQ ID NO: 5 - protein, 87 aa, Homo sapiens
KDPNAPKRPPSAFFLFSSEYRPKIKGEHPGLSIGDVAKKLGEMWNNTAADDKQPYEKKAEKLKEKYEKDIAAYRAKGKPDAAKKGVV

SEQ ID NO: 6 - protein, 97 aa, Homo sapiens
PPKGETKKKFKDPNAPKRPPSAFFLFSSEYRPKIKGEHPGLSIGDVAKKLGEMWNNTAADDKQPYEKKAEKLKEKYEKDIAAYRAKGKPDAAKKGVV

SEQ ID NO: 7 - protein, 21 aa, Homo sapiens
NFELRDKSSRPGRNPKTGDVV

SEQ ID NO: 8 - protein, 21 aa, Homo sapiens
SLHHRQPRLGRNPKTGDSVNL

SEQ ID NO: 9 - protein, 473 aa, synthetic construct
MDPKGSLSWRILLFLSLAFELSYGEVKLVESGGGLVQPGGSLRLSCAASGFTFRTYAMSWVRQAPGKGLEWVATIGSDRRHTYYPDSVKGRFTISRDNAKNTLYLQMNSLRAEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 473 aa, synthetic construct
MDPKGSLSWRILLFLSLAFELSYGEVQLVESGGGLVQPGGSLRLSCAASGFTFRTYAMSWVRQAPGKGLEWVATIGSDRRHTYYPDSVKGRFTISRDNSKNTLYLQMNSLRAEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 473 aa, synthetic construct
MDPKGSLSWRILLFLSLAFELSYGEVKLVQSGAEVKKPGASVKVSCKASGFTFRTYAMSWVRQAPGQRLEWVATIGSDRRHTYYPDKFQGRVTITRDNAKNTLYMELSSLRSEDTAVYYCVGPYDGYYGEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 473 aa, synthetic construct
MDPKGSLSWRILLFLSLAFELSYGEVQLVESGGGLVKPGGSLRLSCAASGFTFSRYGMSWVRQAPGKGLEWVATISSGGSYTYYTDSVKGRFTISRDNAKNSLYLQMNSLRAEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 473 aa, synthetic construct
MDPKGSLSWRILLFLSLAFELSYGEVQLVESGGGLVKPGGSLRLSCAASGFTFSRYGMSWVRQAPGKGLEWVSTISSGGSYTYYTDSVKGRFTISRDNAKNSLYLQMNSLRAEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 473 aa, synthetic construct
MDPKGSLSWRILLFLSLAFELSYGEVQLVESGGGLVQPGRSLRLSCTASGFTFSRYGMSWVRQAPGKGLEWVATISSGGSYTYYTDSVKGRFTISRDNAKNILYLQMNSLKTEDTAVYYCERHGGDGYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 239 aa, synthetic construct
METDTLLLWVLLLWVPGSTGDVVMTQSPLSLPVTLGQPASISCRSSQSLLDSDGKTFLNWLQQRPGQSPRRLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 239 aa, synthetic construct
METDTLLLWVLLLWVPGSTGDVVMTQSPLSLPVTLGQPASISCRSSQSLLDSDGKTFLNWLQQRPGQSPRRLIYLVSKRDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 239 aa, synthetic construct
METDTLLLWVLLLWVPGSTGDVVMTQSPDSLAVSLGERATINCKSSQSLLDSDGKTFLNWLQQKPGQPPKRLIYLVSKLDSGVPDRFSGSGSGTDFTLTISSLQAEDVAVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 233 aa, synthetic construct
METDTLLLWVLLLWVPGSTGDIQMTQSPSSLSASVGDRVTITCRASQDISNYLNWYQQKPGKAVKLLIYYTSRLHSGVPSRFSGSGSGTDYTLTISSLQPEDFATYFCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 233 aa, synthetic construct
METDTLLLWVLLLWVPGSTGDIQMTQSPSSLSASVGDRVTITCRASQDISNYLNWYQQKPGKAVKLLIYYTSRLHSGVPSRFSGSGSGTDYTLTISSLQPEDFATYYCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 233 aa, synthetic construct
METDTLLLWVLLLWVPGSTGDIVMTQSPATLSLSPGERATLSCRASQDISNYLNWYQQKPGQAVRLLIYYTSRLHSGIPARFSGSGSGTDYTLTISSLEPEDFAVYFCQQGNPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 473 aa, synthetic construct
  VARIANT 27: note = Any amino acid, such as Q or K
  VARIANT 30: note = Any amino acid, such as E or Q
  VARIANT 33: note = Any amino acid, such as G or A
  VARIANT 34: note = Any amino acid, such as G or E
  VARIANT 35: note = Any amino acid, such as L or V
  VARIANT 36: note = Any amino acid, such as V or K
  VARIANT 37: note = Any amino acid, such as Q or K, or absent
  VARIANT 40: note = Any amino acid, such as R, G or A
  VARIANT 42: note = Any amino acid, such as L or V
  VARIANT 43: note = Any amino acid, such as R or K
  VARIANT 44: note = Any amino acid, such as L or V
  VARIANT 47: note = Any amino acid, such as T, A or K
  VARIANT 54: note = Any amino acid, such as S or R, or absent
  VARIANT 55: note = Any amino acid, such as R or T, or absent
  VARIANT 57: note = Any amino acid, such as G or A, or absent
  VARIANT 67: note = Any amino acid, such as K or Q
  VARIANT 68: note = Any amino acid, such as G or R
  VARIANT 73: note = Any amino acid, such as A or S
  VARIANT 76: note = Any amino acid, such as S or G, or absent
  VARIANT 78: note = Any amino acid, such as G or D, or absent
  VARIANT 79: note = Any amino acid, such as G or R, or absent
  VARIANT 80: note = Any amino acid, such as S or R, or absent
  VARIANT 81: note = Any amino acid, such as Y or H, or absent
  VARIANT 85: note = Any amino acid, such as T or P, or absent
  VARIANT 87: note = Any amino acid, such as S or K
  VARIANT 88: note = Any amino acid, such as V or F
  VARIANT 89: note = Any amino acid, such as K or Q
  VARIANT 92: note = Any amino acid, such as F or V
  VARIANT 95: note = Any amino acid, such as S or T
  VARIANT 99: note = Any amino acid, such as A or S
  VARIANT 102: note = Any amino acid, such as I, S or T
  VARIANT 105: note = Any amino acid, such as L or M
  VARIANT 106: note = Any amino acid, such as Q or E
  VARIANT 107: note = Any amino acid, such as M or L
  VARIANT 108: note = Any amino acid, such as N or S
  VARIANT 111: note = Any amino acid, such as K or R
  VARIANT 112: note = Any amino acid, such as T, A or S
  VARIANT 121: note = Any amino acid, such as E or V, or absent
  VARIANT 122: note = Any amino acid, such as R or G, or absent
  VARIANT 123: note = Any amino acid, such as H or P, or absent
  VARIANT 124: note = Any amino acid, such as G or Y, or absent
  VARIANT 125: note = Any amino acid, such as G or D, or absent
  VARIANT 126: note = Any amino acid, such as D or G, or absent
  VARIANT 127: note = Any amino acid, such as G or Y, or absent
  VARIANT 129: note = Any amino acid, such as W or G, or absent
  VARIANT 130: note = Any amino acid, such as Y or E, or absent
  VARIANT 133: note = Any amino acid, such as V or Y, or absent
  VARIANT 139: note = Any amino acid, such as M or L, or absent
MDPKGSLSWRILLFLSLAFELSYGEVQLVESGGGLVXPGGSLRLSCAASGFTFXXYXMSWVRQAPGKGLEWVATIXSXXXXTYYXDSVKGRFTISRDNAKNTLYLQMNSLRAEDTAVYYCXXXXXXXYXXFDXWGQGTXVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 239 aa, synthetic construct
  VARIANT 22: note = Any amino acid, such as V or I, or absent
  VARIANT 23: note = Any amino acid, such as V or Q
  VARIANT 29: note = Any amino acid, such as L, D, S, or A, or absent
  VARIANT 30: note = Any amino acid, such as S or T
  VARIANT 32: note = Any amino acid, such as S, P, or A
  VARIANT 33: note = Any amino acid, such as V, A, or L
  VARIANT 34: note = Any amino acid, such as S or T
  VARIANT 35: note = Any amino acid, such as L, V, or P
  VARIANT 37: note = Any amino acid, such as Q, E, or D, or absent
  VARIANT 38: note = Any amino acid, such as R or P
  VARIANT 39: note = Any amino acid, such as A or V
  VARIANT 40: note = Any amino acid, such as T or S
  VARIANT 41: note = Any amino acid, such as I or L
  VARIANT 42: note = Any amino acid, such as S, N, or T
  VARIANT 44: note = Any amino acid, such as R or K
  VARIANT 45: note = Any amino acid, such as S or A, or absent
  VARIANT 48: note = Any amino acid, such as S or D, or absent
  VARIANT 49: note = Any amino acid, such as L or I, or absent
  VARIANT 50: note = Any amino acid, such as L or S, or absent
  VARIANT 51: note = Any amino acid, such as D or N, or absent
  VARIANT 52: note = Any amino acid, such as S or Y, or absent
  VARIANT 53: note = Any amino acid, such as D or L, or absent
  VARIANT 54: note = Any amino acid, such as G or N, or absent
  VARIANT 55: note = Any amino acid, such as K or W, or absent
  VARIANT 56: note = Any amino acid, such as T or Y, or absent
  VARIANT 57: note = Any amino acid, such as F or Q, or absent
  VARIANT 61: note = Any amino acid, such as L, or absent
  VARIANT 64: note = Any amino acid, such as K or R
  VARIANT 67: note = Any amino acid, such as Q or K
  VARIANT 68: note = Any amino acid, such as A, S, or P
  VARIANT 69: note = Any amino acid, such as P or V, or absent
  VARIANT 70: note = Any amino acid, such as R or K, or absent
  VARIANT 71: note = Any amino acid, such as R or L, or absent
  VARIANT 75: note = Any amino acid, such as L or Y, or absent
  VARIANT 76: note = Any amino acid, such as V or T, or absent
  VARIANT 78: note = Any amino acid, such as K or R, or absent
  VARIANT 79: note = Any amino acid, such as L or R
  VARIANT 80: note = Any amino acid, such as D or H, or absent
  VARIANT 83: note = Any amino acid, such as V or I
  VARIANT 85: note = Any amino acid, such as D, S, or A
  VARIANT 96: note = Any amino acid, such as F or Y, or absent
  VARIANT 99: note = Any amino acid, such as T or K
  VARIANT 102: note = Any amino acid, such as S or R
  VARIANT 103: note = Any amino acid, such as L or V
  VARIANT 104: note = Any amino acid, such as E or Q, or absent
  VARIANT 105: note = Any amino acid, such as A or P, or absent
  VARIANT 108: note = Any amino acid, such as V or F, or absent
  VARIANT 109: note = Any amino acid, such as A or G
  VARIANT 110: note = Any amino acid, such as V or T
  VARIANT 112: note = Any amino acid, such as Y or F
  VARIANT 114: note = Any amino acid, such as W or Q, or absent
  VARIANT 117: note = Any amino acid, such as T or N, or absent
  VARIANT 118: note = Any amino acid, such as H or P, or absent
  VARIANT 119: note = Any amino acid, such as F or L, or absent
  VARIANT 120: note = Any amino acid, such as P or R, or absent
  VARIANT 121: note = Any amino acid, such as Y, or absent
  VARIANT 125: note = Any amino acid, such as Q or G, or absent
  VARIANT 129: note = Any amino acid, such as L or V, or absent
METDTLLLWVLLLWVPGSTGDXVMTQSPXSLSVSLGXRATISCRXSQXXXXXXXXXXLNWXQQKPGQAXXXLIYXXSXLXSGVPDRFSGSGSGTDXTLTISSLXXEDXAVYYCXQGXXXXXTFGXGTKXEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 330 aa, Homo sapiens [sequence omitted]

SEQ ID NO: 24 - protein, 473 aa, synthetic construct; identical to SEQ ID NO: 9
  VARIANT 27: note = K or Q
  VARIANT 30: note = E or Q
  VARIANT 33: note = G or A
  VARIANT 34: note = G or E
  VARIANT 35: note = L or V
  VARIANT 36: note = V or K
  VARIANT 37: note = Q or K
  VARIANT 40: note = G or A
  VARIANT 42: note = L or V
  VARIANT 43: note = R or K
  VARIANT 44: note = L or V
  VARIANT 47: note = A or K
  VARIANT 67: note = K or Q
  VARIANT 68: note = G or R
  VARIANT 87: note = S or K
  VARIANT 88: note = V or F
  VARIANT 89: note = K or Q
  VARIANT 92: note = F or V
  VARIANT 95: note = S or T
  VARIANT 99: note = A or S
  VARIANT 105: note = L or M
  VARIANT 106: note = Q or E
  VARIANT 107: note = M or L
  VARIANT 108: note = N or S
  VARIANT 112: note = A or S

SEQ ID NO: 25 - protein, 239 aa, synthetic construct; identical to SEQ ID NO: 15
  VARIANT 29: note = D or L
  VARIANT 32: note = A or P
  VARIANT 34: note = S or T
  VARIANT 37: note = E or Q
  VARIANT 38: note = R or P
  VARIANT 40: note = T or S
  VARIANT 42: note = N or S
  VARIANT 44: note = K or R
  VARIANT 64: note = K or R
  VARIANT 68: note = P or S
  VARIANT 70: note = K or R
  VARIANT 79: note = L or R
  VARIANT 99: note = T or K
  VARIANT 102: note = S or R
  VARIANT 103: note = L or V
  VARIANT 104: note = Q or E
  VARIANT 109: note = A or G

SEQ ID NO: 26 - protein, 60 aa, synthetic construct
  VARIANT 7: note = Any amino acid
  VARIANT 21..40: note = Any amino acid
  VARIANT 21..40: note = This region may encompass 1-20 residues
  VARIANT 55: note = Any amino acid
RPGRNPXTGDVVPVSARRVVXXXXXXXXXXXXXXXXXXXXFSLHHRQPRLGRNPXTGDSV

SEQ ID NO: 27 - protein, 60 aa, synthetic construct
  VARIANT 21..40: note = Any amino acid
  VARIANT 21..40: note = This region may encompass 1-20 residues
RPGRNPKTGDVVPVSARRVVXXXXXXXXXXXXXXXXXXXXFSLHHRQPRLGRNPKTGDSV

SEQ ID NO: 28 - protein, 44 aa, synthetic construct
RPGRNPKTGDVVPVSARRVVGPSLFSLHHRQPRLGRNPKTGDSV

SEQ ID NO: 29 - protein, 137 aa, Escherichia coli
MSEALKILNNIRTLRAQARECTLETLEEMLEKLEVVVNERREEESAAAAEVEERTRKLQQYREMLIADGIDPNELLNSLAAVKSGTKAKRAQRPAKYSYVDENGETKTWTGQGRTPAVIKKAMDEQGKSLDDFLIKQ

SEQ ID NO: 30 - protein, 134 aa, Haemophilus influenzae
MNELVRGLTNLRSLRAAVRELTLEQAENALEKLQTAIEEKRANEAELIKAETERKERLAKYKELMEKEGITPEELHKIFGTKTVSIQAKRAPRPAKYAFIDENGEHKTWTGQGRTPRPIQNALNKGKSLSDFEI

SEQ ID NO: 31 - protein, 134 aa, Mycobacterium tuberculosis
MPDPQDRPDSEPSDASTPPAKKLPAKKAAKKAPARKTPAKKAPAKKTPAKGAKSAPPKPAEAPVSLQQRIETNGQLAAAAKDAAAQAKSTVEGANDALARNASVPAPSHSPVPLIVAVTLSLLALLLIRQLRRR

SEQ ID NO: 32 - protein, 134 aa, Streptococcus pneumoniae
MNELVRSLTNLRSLRAAVRELTLEQAENALEKLQTAIEEKRANEAELIKAETERKERLAKYKELMEKEGITPEELHEIFGTKTVSIRAKRPPRPAKYAFIDENGEHKTWTGQGRTPRPIQNALNKGKSLSDFEI

SEQ ID NO: 33 - protein, 135 aa, Klebsiella pneumoniae
MSEALKILNNIRTLRAQARECTLETLEEMLEKLEVVVNERREEENAAAAEIEERTRKLQQYREMLIADGIDPNELLSTMAAVKAGTKTKRAARPAKYSYVDENGETKTWTGQGRTPAVIKKAMDEQGKSLDDFLI

SEQ ID NO: 34 - protein, 120 aa, Pseudomonas aeruginosa
MSLINEYRNTEQTIKELQARLASLQQDGRMKAELEFDTKLRALMSEYNKSLRDVIILLDPQAQNRSSKTPPTSGRRERQLKRYLNPNTSEVVETKGGNHKILKEWKTQFGADVVESWLQS

SEQ ID NO: 35 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 36 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 37 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 38 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 39 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 41 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 42 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 60 aa, synthetic construct; identical to SEQ ID NO: 26
  VARIANT 7: note = Any amino acid
  VARIANT 21..40: note = Any amino acid
  VARIANT 21..40: note = This region may encompass 1-20 residues
  VARIANT 55: note = Any amino acid

SEQ ID NO: 44 - protein, 60 aa, synthetic construct; identical to SEQ ID NO: 27
  VARIANT 21..40: note = Any amino acid
  VARIANT 21..40: note = This region may encompass 1-20 residues

SEQ ID NO: 45 - protein, 44 aa, synthetic construct; identical to SEQ ID NO: 28

SEQ ID NO: 46 - protein, 60 aa, synthetic construct
  VARIANT 21..40: note = Any amino acid
  VARIANT 21..40: note = This region may encompass 1-20 residues
FLEEIRLSLESGQDVKLSGFXXXXXXXXXXXXXXXXXXXXTLSAKEIENMVKDILEFISQ

SEQ ID NO: 47 - protein, 7 aa, synthetic construct
GFTFRTY

SEQ ID NO: 48 - protein, 6 aa, synthetic construct
GSDRRH

SEQ ID NO: 49 - protein, 13 aa, synthetic construct
VGPYDGYYGEFDY

SEQ ID NO: 50 - protein, 44 aa, synthetic construct
FLEEIRLSLESGQDVKLSGFGPSLTLSAKEIENMVKDILEFISQ

SEQ ID NO: 51 - protein, 11 aa, synthetic construct
QSLLDSDGKTF

SEQ ID NO: 52 - protein, 7 aa, synthetic construct
WQGTHFP

SEQ ID NO: 53 - protein, 8 aa, synthetic construct
GFTFSRYG

SEQ ID NO: 54 - protein, 8 aa, synthetic construct
ISSGGSYT

SEQ ID NO: 55 - protein, 13 aa, synthetic construct
ERHGGDGYWYFDV

SEQ ID NO: 56 - protein, 9 aa, synthetic construct
WQGTHFPYT

(19) United States
(12) Patent Application Publication
(10) Pub. No.: US 2026/0152531 A1
(43) Pub. Date: Jun. 4, 2026

(54) COMPOSITIONS AND METHODS FOR TREATING BIOFILMS AND NEUTROPHIL EXTRACELLULAR TRAP FORMATION

(71) Applicant: RESEARCH INSTITUTE AT NATIONWIDE CHILDREN'S HOSPITAL, Columbus, OH (US)

(72) Inventors: Steven D. Goodman, Columbus, OH (US); Lauren O. Bakaletz, Columbus, OH (US); Santiago Partida-Sanchez, Columbus, CA (US)

(21) Appl. No.: 19/126,589

(22) PCT Filed: Nov. 8, 2023
(86) PCT No.: PCT/US2023/079162

§ 371 Date: May 1, 2025

Related U.S. Application Data
(60) Provisional application No. 63/424,851, filed on Nov. 11, 2022.
(60) Provisional application No. 63/424,888, filed on Nov. 12, 2022.

(51) Int. Cl. C07K 14/005; C07K 16/00

(52) U.S. Cl. CPC C07K 14/4702; A61K 38/164; A61P 31/04; C07K 16/12; C12N 15/11; C07K 2317/565

(57) ABSTRACT

Provided herein is a synthetic polypeptide derived from High Mobility Group Box 1 (HMGB 1) host protein that can both disrupt bacterial biofilms and prevent Neutrophil Extracellular Trap (NET) formation. Also provided herein are methods to disrupt aberrant or excessive NET formation that are particularly well-suited to treat high-risk populations such as those infected with SARS CoV-2, sepsis, autoimmune diseases e.g., systemic lupus erythematosus, rheumatoid arthritis, Type I diabetes mellitus, small vessel vasculitis, autoinflammatory diseases e.g., gout, inflammatory bowel disease, and metabolic diseases e.g., Type 2 diabetes and obesity.

### CROSS-REFERENCE TO RELATED PATENT APPLICATIONS

This application is a national stage application under 35 U.S.C. 371 of PCT Application No. PCT/US2023/079162, filed Nov. 8, 2023, which in turn claims priority under 35 U.S.C. § 119(e) of U.S. Provisional Application No. 63/424,851, filed Nov. 11, 2022, and U.S. Provisional Application No. 63/424,888, filed Nov. 12, 2022, the entire contents of which are incorporated herein by reference.

### STATEMENT OF GOVERNMENT SUPPORT

This invention was made with government support under AII55501 & DC011818 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Mar. 22, 2024, is named 106887-9260_SL.xml and is 425,872 bytes in size.

### BACKGROUND

Neutrophils (Polymorphonuclear leukocytes or PMNs) are specialized cells that are part of the innate immune system. Their function is, in part, to migrate to sites of infection to clear pathogens. One method PMNs can use is a process known as NETosis to create elaborate structures (neutrophil extracellular traps or NETs) of released extracellular DNA (eDNA) that first ensnare individual bacteria, cordon off bacterial communities (biofilms) and to use the deployed eDNA tendrils localize and focus NET-associated antimicrobial substances towards the ensnared microorganisms, thus limiting pathogen proliferation.

Despite this critical host innate immune function, widespread release of NETs can occur in the NET's attempt to clear bacterial and viral pathogens, including pathological blood clots that can lead to morbidity or even death. In addition, NETs can induce a counter-inflammatory immune response (e.g., autoimmune diseases). Thus, development of a means to prevent or disable the eDNA structure of NETs to prevent or arrest these potentially damaging clotting or excessive inflammatory events is desirable. This disclosure satisfies this need and provides related advantages as well.

### SUMMARY OF THE DISCLOSURE

Applicant has discovered that a polypeptide derived from High Mobility Group Box 1 (HMGB1) host protein can both disrupt bacterial biofilms and prevent Neutrophil Extracellular Trap (NET) formation. This polypeptide, termed by Applicant as “mB Box-97” consists essentially of or consists of the amino acids 80 to 176 from the coding sequence of the native human HMGB1 protein with a cysteine to serine point mutation at amino acid 106 that abrogates the ability of this polypeptide to induce an inflammatory response. Native HMGB1 is known to be variably altered by the host through post translational modifications and as such these changes have been shown to affect HMGB1 function. Likewise, mB Box-97 should also be subject to post translational modifications when expressed recombinantly by bacteria. To that point, Applicant synthesized mB Box-97 and found that both recombinant and synthetic mB Box-97 had indistinguishable biofilm disruption and NET inhibition activities. Thus, synthetic mB Box-97 can be synthesized for therapeutic use, improving yield and quality control over recombinant mB Box-97 and mutated wild-type polypeptides without loss of therapeutic activity. Thus, the synthetically produced mb Box-97 with the cysteine to serine point mutation of amino acid is termed herein as “synthetic mB Box-97 or smB Box-97.”

Applicant's disclosure also addresses and solves problems associated with aberrant or excessive NET formation particularly in high-risk populations such as those infected with SARS CoV-2, sepsis, autoimmune diseases e.g., Systemic Lupus Erythematosus, Rheumatoid arthritis, Type I diabetes mellitus, small vessel vasculitis, autoinflammatory diseases e.g., Gout, Inflammatory Bowel Disease, and metabolic diseases e.g. Type 2 diabetes and obesity. Without being bound by theory, the mB Box 97 can prevent NET-mediated disease or prevent the progression of NET-mediated disease. Second, synthetic mB Box-97 can be used to prevent or treat bacterial biofilms because the mechanism of action of this peptide is distinct from other known similar peptides.

In another aspect of this disclosure, Applicant has discovered that DNA-binding proteins that aggregate DNA can also condense the eDNA tendrils of NETs, either preventing NET formation or inducing retraction of extant NETs. For example, the family of homologous bacterial Histone-like Nucleoid Structuring protein (H-NS) proteins that are known to function as nucleoid associated proteins, intracellularly, have a heretofore unknown additional effect extracellularly. These H-NS proteins are released from bacterial biofilms and not only prevent NET formation but also collapse extant NETs. While the ability to bridge adjacent DNA duplexes is a known function of H-NS, it was unknown that this level of DNA aggregation/condensation was also a property. Thus, any agent that causes DNA aggregation/condensation can also be used to limit conditions of counter effective NETosis e.g. those associated with SARS CoV-2 infection.

There are no FDA approved preventatives or treatments for pathological NETosis. There are therapeutics that may prevent NETosis, inactivate NETs or facilitate clearance of NETs (reviewed in Mutua and Gershwin (2020) Clinical Reviews in Allergy and Immunology 61: 194-211; https://pubmed.ncbi.nlm.nih.gov/32740860/, incorporated herein by reference). The biggest disadvantages of inactivating NETs is that NETs also possess beneficial functions. Virtually all approaches to treat NETs fail to compensate for the loss of beneficial functions. However, DNA aggregating agents can be used to treat extant NETs such that only NET pathology is affected.

This disclosure solves the problems associated with aberrant or excessive NET formation particularly in high-risk populations such as those infected with SARS CoV-2, sepsis, autoimmune diseases, e.g., Systemic Lupus Erythematosus, Rheumatoid arthritis, Type I diabetes mellitus, small vessel vasculitis, autoinflammatory diseases, e.g., Gout, Inflammatory Bowel Disease, and metabolic diseases, e.g., Type 2 diabetes and obesity. These DNA aggregating agents could be used to prevent or treat NET-mediated disease or prevent the progression of NET-mediated disease.

### BRIEF DESCRIPTION OF THE DRAWINGS

FIGS. 1A-1C. mB Box-97 (also known as mB Box-97 peptide) inhibits NETosis induced by Phorbol Myristate Acetate (PMA). (A) Graphical representations of Recombinant or synthetic full-length HMGB1, ABox (amino acids 1 to 89), ABBox (amino acids 1 to 176), BBox-97 (amino acids 80 to 176), recombinant and synthetic mB Box-97 (identical to BBox-97 except for a single amino acid change C106S) and BBox-87 (amino acids 90 to 176) polypeptides. Specific Cysteine/C and its respective amino acid number are shown, in mB Box-97 C at corresponding position 106 on HMGB1 is mutated to Serine/S. Recombinantly produced HMGB1 polypeptides (e.g., full length HMGB1, ABox, ABBox, BBox-97, mB Box-97, BBox-87) may have post-translational modifications, whereas synthetic HMGB1 polypeptides do not have any post-translational modifications. (B) Quantification of fluorescence studies show that mB Box-97, BBox-87, and ABox do not induce NETosis. Nets were induced in isolated neutrophils (5*10³) using respective constructs (200 nm, 4 hr), and released DNA was quantified using cell impermeable dye SYTOX green (1 μM) using a fluorometer. Out of all constructs tested, mB Box-97, BBox-87 and ABox were unable to induce NETs formation in isolated human neutrophils. (C) Quantification of NETs % of total cells in different treatment conditions shows only mB Box-97 having significant inhibition of PMA-induced NETosis. Formed NETs were quantified using ImageJ and the % NET formation was calculated against total neutrophils in each image. An analysis from 4 different experiments is shown, and the data is presented as mean f standard deviation (SD). *P<0.05 as assessed by unpaired t-test. mB Box-97 was the only HMGB1 construct that showed inhibition of PMA-induced NETosis.

FIGS. 2A-2F. Ca²⁺ mediates NETosis following an alternate pathway to that of PMA or LPS, therefore Applicant investigated the effect of mB Box-97 on the Ca²⁺ mediated NETs formation. Neutrophils were allowed to form NETs in the presence of Ca²⁺ ionophore A23187 (500 nM) with or without mB Box-97 or BBox-97 (200 nM) for 6-8 hrs. DNA, Plasma membrane, NE or MPO were probed as described in the material and methods and NETs were visualized by CLSM at 63× magnification. (A) Changes in Mean Fluorescence Intensity (MFI) of the NETs associated proteins upon PMA-stimulated NETosis. (B) Changes in Mean Fluorescence Intensity (MFI) of the NETs associated proteins upon NTHI-stimulated NETosis. (C) Changes in Mean Fluorescence Intensity (MFI) of the NETs associated proteins upon Ionophore-stimulated NETosis. (D) mB Box-97 inhibits the secretion of NETs-associated protein with the inhibition of NETosis induced by PMA or NTHI. (E) Secreted nuclear elastase (NE) measurements. (F) DNA-bound NE measurements.

FIGS. 3A-3B. Effect of mB Box-97 on the generation of Reactive Oxygen Species (ROS) and phosphorylation of p47^{phox}. (A) Effect of mB Box-97 on ROS generation. Neutrophils preincubated with Luminol (400 μM) were incubated with either PMA, PMA with or without proteins, proteins or with buffer alone, and luminescence as a measure of ROS generated was detected over time for 2 hrs at intervals of 5 min. N=3, bars represent the SEM. *P<0.007 or lower as assessed by multiple unpaired t-test. (B) Effect of mB Box-97 on p47^{phox} phosphorylation. Neutrophils (1×10⁷ cells/ml) were incubated with buffer, PMA 200 nM, or PMA with or without mB Box-97 or BBox-97 (1 μM) for 30 min and lysed, and proteins were separated with SDS-PAGE and transferred to nitrocellulose membrane. Proteins were detected using immunoblotting with anti-phospho-p47^{phox} antibody detecting phosphorylation on Ser-370, or anti-p47^{phox} or GAPDH. Western blots from different experiments were scanned; phosphorylated and total p47^{phox} and GAPDH were quantified by densitometry; and the intensity of phosphorylated p47phox was corrected for the amount of p47^{phox} after correcting both for the amount of GAPDH. Results are expressed as mean±SD (n=4). ****P<0.0001 as assessed by unpaired t-test. mB Box-97 significantly inhibited ROS production as well as phosphorylation of key NOX protein p47^{phox} in human neutrophils upon induction of NETosis using PMA.

FIGS. 4A-4B. (A) Neutrophil mediated killing is inactivated by mB Box-97. 16 hr NTHI biofilms were challenged with human neutrophils 10⁶ (B+N) and treated with 1 μM recombinant DNABII protein HU_{NTHI} (B+N+HU) positive control, 1 μM mB Box-97 (B+N+mB Box-97) or 1 μM BBox-97 (B+N+BBox-97) for 4 h. NTHI biofilm without neutrophils was used as a control. The bacteria challenged with neutrophils (B+N) (mean=50.79%) and the group treated with BBox-97 (B+N+BBox-97) (mean=40.86%) showed an increase in the relative % killing as compared to the biofilm control. The neutrophils treated with HU_{NTHI} (B+N+HU) (mean=4.83%) and mB Box-97 (B+N+mB Box-97) (mean=13.09%) did not show differences in the relative % killing of bacteria by neutrophils. The results suggest that mB Box-97 treatment inactivated the neutrophil mediated bacterial killing. The graph plot replicates were derived from 6 healthy donors±SEM. The statistical analysis was performed with one-way ANOVA and Dunnett's multiple comparison test (**p<0.01). mB Box-97 inhibits bacterial killing mediated by neutrophils which was at par with inhibition by DNABII protein from NTHI, HU_{NTHI}. (B) ROS production measurements. Preloaded Neutrophils with Luminol were incubated with either PMA, PMA with or without respective proteins, or with buffer alone, and luminescence as a measure of ROS was detected over time for 2 hrs at intervals of 5 min.

FIG. 5. Interaction and inhibition of PKC with mB Box-97. mB Box-97 inhibits PKC activity. The activity of PKC was tested in the presence of Histone H1, mB Box-97 and BBox-97 in two different concentrations (200 and 1000 nM) using a PKC activity assay kit as per the manufacturer's instruction. mB Box-97 shows inhibition of PKC activity in increasing concentration.

FIG. 6. Graphical representation of a potential mechanism for mB Box-97 action. mB Box-97 interacts with PKC leading to the inhibition of its activity. This inhibition leads to the inhibition of phosphorylation of p47^{phox} therefore inhibiting of active assembly of NOX thereby reducing the generation of ROS. Reduction in ROS leads to a reduction in the release of NE and MPO which ultimately leads to the inhibition of NETs formation. mB Box-97 also partially inhibits Ca²⁺-induced NETosis probably through inhibition of PKC activity as Ca2+ also influences the PKC activity.

FIGS. 7A-7B. (A) Biofilm disruptive abilities of HMGB1-derived peptides. (B) mB Box-97_{syn} significantly disrupts four additional high priority ESKAPEE pathogens (an acronym for Enterococcus faecium, Staphylococcus aureus, Klebsiella pneumoniae, Acinetobacter baumannii, Pseudomonas aeruginosa, Enterobacter spp., and Escherichia coli) regardless of biofilm age.

FIGS. 8A-8B. mB Box-97 prevents lung infection. Adult C57BL/6 mice with (A) 10⁷ or (B) 10⁸ CFU of B. cenocepacia (Bc) intratracheally (i.t.), along with either 200 nM of mB Box-97 or a negative control.

FIGS. 9A-9B. (A) mB Box 97 or HuTipMab (“HuTipMab” is an antibody as described in Kurbatfinski, N. et al. Antimicrobial Agents and Chemotherapy 66.3 (2022): e01877-21 and WO2021007260A2) significantly inhibits biofilm growth by both NTHI and S. aureus compared to negative controls. (B) mB Box-97 (P=0.03-P<0.0001) or HuTipMab (P=0.02-P<0.0001) prevents biofilm growth upon 16 h incubation of P. aeruginosa (Pa), Enterobacteriaceae sp (Esp), E. faecium (Ef), uropathogenic E. coli (UPEC), A. baumannii (Ab) or B. cenocepacia (Bc) compared to negative controls.

FIGS. 10A-10B. mB Box 97 or HuTipMab shows preventative activity on pathogen K. pneumoniae. (A) Biomass measurements, and (B) corresponding representative Confocal Scanning Laser Microscopy (CSLM) images.

FIGS. 11A-11B. mB Box-97 and HuTipMab show synergy in inhibiting biofilm formation. (A) Prevention of NTHI biofilms by dilutions of mB Box-97, HuTipMab, or both together. (B) Prevention of S. aureus biofilms by dilutions of mB Box-97 or HuTipMab.

FIG. 12. Graphical representation showing that H-NS prevents killing of bacteria by Neutrophil Extracellular Traps.

FIGS. 13A-13B. H-NS protein levels decrease within NTHI, S. pneumoniae, and UPEC biofilms as the biofilm matures. (A) Representative confocal images of H-NS (gray) in Streptococcus pneumoniae, NTHI, and UPEC biofilms of ages varying from 24 hours to 1 week. (B) The ratio of fluorescence intensity of H-NS to cells in NTHI, UPEC, and S. pneumoniae in 24, 40, 72 hr, and 1 week old biofilms. An increase was seen from 24 hrs to 40 hrs after which a decrease in that ratio of fluorescence intensity.

FIG. 14. H-NS exits the biofilm and enters the bulk media. The concentration of H-NS was quantified using a Western Blot relative to the CFUs within the biofilm. The concentration was found in the supernatant of the biofilm and within the biofilm at 16 hours and 1 week. The concentration of H-NS increased significantly from 16 hrs to 1 week in the supernatant, but in the biofilm a decrease of H-NS was observed.

FIGS. 15A-15B. H-NS both prevents PMA-induced NET formation and induces condensation of NET-deployed eDNA. (A) Human neutrophils were induced with PMA and with or without H-NS NTHI for 3.5 hrs or (B) induced with PMA for 16 h and then incubated with H-NS for 2 hrs. NETs were incubated with wheat germ agglutinin, α-B-DNA, and α-NE antibodies, and visualized by IF CLSM. H-NS both prevented PMA induced NET formation (A) and induced condensation of NET-deployed eDNA (B).

FIG. 16. H-NS prevented NET killing of NTHI bacteria similar to HU. NTHI biofilms that were 16 hrs old were incubated with human neutrophils for 3 hrs and the percentage of bacteria killed were found relative to the CFUs present in NTHI without neutrophils added. The three proteins added to the NTHI biofilm with neutrophils were HU, CbpA, and H-NS. A prevention of bacterial killing by NETs was achieved by the presence of HU and H-NS. N=5, P<0.05.

### DETAILED DESCRIPTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration and molecular weight, including ranges, are approximations which are varied (+) or (−) by increments of 1.0 or 0.1, as appropriate, or alternatively by a variation of +/−15%, or alternatively 10%, or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure. Embodiments defined by each of these transition terms are within the scope of this disclosure.

As used herein, the term “comprising” is intended to mean that the peptides recited herein include the recited amino acid sequences, but do not exclude other amino acids. “Consisting essentially of” when used to define sequences, refers to a core sequence optionally surrounded by additional amino acids. Thus, a peptide consisting essentially of a sequence as defined herein would not exclude additional amino acids at the C or N terminal. In some embodiments, a peptide consisting essentially of a sequence as defined herein comprises up to ten additional amino acids (e.g., 1, 2, 3, 4, 5, 6, 7, 8, 9, or 10) at the N terminal. In some embodiments, a peptide consisting essentially of a sequence as defined herein comprises up to ten additional amino acids at the C terminal. In some embodiments, a peptide consisting essentially of a sequence as defined herein comprises up to ten additional amino acids at the N terminal and up to ten additional amino acids at the C terminal. As used for a peptide, “consisting of” a sequence means that the peptide does not contain any additional sequences. Embodiments defined by each of these transition terms are within the scope of this disclosure.

A “biofilm” intends a thin layer or an organized community of microorganisms that at times can adhere to the surface of a structure, that may be organic or inorganic, or be concentrated at an interface (usually solid/liquid), surrounded by an extracellular polymeric slime matrix that comprise polymers, such as DNA, that the microorganisms secrete and/or release. Biofilms can contain many different types of micro-organisms, e.g. bacteria, archaea, protozoa, fungi and algae. Biofilms are very resistant to microbiotics and antimicrobial agents. They live on gingival tissues, teeth and restorations, causing caries and periodontal disease, also known as periodontal plaque disease. They also cause chronic middle ear infections. Biofilms can also form on the surface of dental implants, stents, catheter lines and contact lenses. They grow on pacemakers, heart valve replacements, artificial joints and other surgical implants. The Centers for Disease Control estimate that over 65% of nosocomial (hospital-acquired) infections are caused by biofilms. Fungal biofilms also frequently contaminate medical devices. They cause chronic vaginal infections and lead to life-threatening systemic infections in people with hobbled immune systems. Biofilms also are involved in numerous diseases. For instance, cystic fibrosis patients have Pseudomonas infections that often result in antibiotic resistant biofilms.

As used herein, “treatment of a biofilm-related disorder”, also referred to herein as a “for preventing or treating a bacterial biofilm”, refers to a reduction in the severity and/or duration of the disorder and/or a reduction of the severity and/or duration of symptoms from the disorder, in particular the symptoms of infection. In some embodiments, treatment results in restoration of the health of an individual. Preferably, the individual has less severe disease symptoms or for a shorter time. Disease symptoms can be monitored using convention techniques.

In some embodiments, the compositions of the present disclosure can be used for prevention or reduction of biofilm formation or growth in vitro, ex vivo, or in vivo. As used herein, “prevention or reduction of biofilm formation or growth” refers to the prevention, delay, or reduction of biofilm formation or growth in vitro, ex vivo, or in vivo. As will be understood by a skilled person, such reduction of biofilm formation or growth can slow the growth of biofilms as compared to the growth of untreated biofilms.

In some embodiments the compositions disclosed herein are useful for degrading or reducing biofilms. As used herein, “degrading or reducing biofilms” refers to the elimination, either partially or completely, of a biofilm in vitro, ex vivo, or in vivo. As will be understood by a skilled person, after such treatment, planktonic bacteria (i.e., free-living bacteria that are suspended in liquid) may still be present.

A “DNABII polypeptide or protein” intends a DNA binding protein or polypeptide that is composed of DNA-binding domains and thus have a specific or general affinity for DNA. In one aspect, they bind DNA in the minor grove. Non-limiting examples of DNABII proteins are an integration host factor (HF) protein and a histone-like protein from E. coli strain U93 (HU). Other DNA binding proteins that can be associated with the biofilm include DPS (Genbank Accession No.: CAA49169), H-NS (Genbank Accession No.: CAA47740), Hfq (Genbank Accession No.: ACE63256), CbpA (Genbank Accession No.: BAA03950) and CbpB (Genbank Accession No.: NP_418813).

A “tip fragment” of a DNABII polypeptide intends a DNABII polypeptide that, using IHFalpha and IHFbeta as examples, forms the two arms of the proteins. Non-limiting examples of such include IhfA, A tip fragment: NFELRDKSSRPGRNPKTGDVV, SEQ ID NO: 7, and IhfB, B tip fragment: SLHHRQPRLGRNPKTGDSVNL, SEQ ID NO: 8.

As used herein, the term “tip-chimeric peptide” or “an IhfA5-mIhfB4_{NTHI} tip chimer” or a “tip chimer” intends a chimer of an IhfA5-mIhfB4_{NTHI} peptide that comprises, or consists essentially of, or yet further consists of: a polypeptide sequence of RPGRNPX₁TGDVVPVSARRVV-X-FSLHHRQPRLGRNPX₁TGDSV (SEQ ID NO: 43), wherein “X” is an optional amino acid linker sequence, optionally comprising, or consisting essentially of, or yet further consisting of between 1 to 20 amino acids; and wherein “X₁” is any amino acid or alternatively “X₁” is selected from the amino acids Q, R, K, S, or T. In a Rfurther aspect, “X₁” is a K or Q. In a further embodiment, the tip-chimeric peptide IhfA5-mIhfB4_{NTHI} comprises, or consists essentially of, or yet further consists of: a polypeptide sequence of RPGRNPKTGDVVPVSARRVV-X-FSLHHRQPRLGRNPKTGDSV (SEQ ID NO: 44), wherein “X” is an optional amino acid linker sequence optionally comprising, or consisting essentially of, or yet further consisting of between 1 to 20 amino acids. In yet a further embodiment, the tip-chimeric peptide IhfA5-mIhfB4 mm comprises or consists essentially of, or yet further consists of: a polypeptide sequence of RPGRNPKTGDVVPVSARRVVGPSLFSLHHRQPRLGRNPKTGDSV (SEQ ID NO: 45).

In certain embodiments, the tail-chimeric peptide IhfA3-IhfB2_{NTHI} comprises, or consists essentially of, or yet further consists of: a polypeptide sequence of FLEEIRLSLESGQDVKLSGF-X-TLSAKEIENMVKDILEFISQ (SEQ ID NO: 46), wherein “X” is an optional amino acid linker sequence optionally comprising, or consisting essentially of, or yet further consisting of between 1 to 20 amino acids. In certain embodiments, the linker is selected from any one or more of SEQ ID NOs: 3-4. In one embodiment, the tail-chimeric peptide IhfA3-IhfB2_{NTHI} comprises, or consists essentially of, or yet further consists of FLEEIRLSLESGQDVKLSGFGPSLTLSAKEIENMVKDILEFISQ (SEQ ID NO: 50).

An “integration host factor” or “IHF” protein is a bacterial protein that is used by bacteriophages to incorporate their DNA into the host bacteria. These are DNA binding proteins that function in genetic recombination as well as in transcription and translational regulation. They also bind extracellular microbial DNA. The genes that encode the IHF protein subunits in E. coli are himA (Genbank accession No.: POA6X7.1) and himD (POA6Y1.1) genes.

“HMGB1” is a high mobility group box (HMGB) 1 protein that is reported to bind to and distort the minor groove of DNA and is an example of an interfering agent. Recombinant or isolated protein and polypeptide are commercially available from Atgenglobal, ProSpecBio, Protein1 and Abnova. The sequences of wild type murine HMGB1 and human HMGB1 protein is provided in the Sequence Listing as SEQ ID NO: 1 and SEQ ID NO: 2, respectively.

“HU” or “histone-like protein from E. coli strain U93” refers to a class of heterodimeric proteins typically associated with E. coli. HU proteins are known to bind DNA junctions. Related proteins have been isolated from other microorganisms. The complete amino acid sequence of E. coli HU was reported by Laine et al. (1980) Eur. J. Biochem. 103(3):447-481. Antibodies to the HU protein are commercially available from Abcam.

A “linker” or “peptide linker” refers to a peptide sequence linked to either the N-terminus or the C-terminus of a polypeptide sequence. In one aspect, the linker is from about 1 to about 20 amino acid residues long or alternatively 2 to about 10, about 3 to about 5 amino acid residues long. Examples of peptide linkers are Gly-Pro-Ser-Leu-Lys-Leu (SEQ ID NO: 3) and PPKGETKKKF (SEQ ID NO: 4).

The term “Haemophilus influenzae” refers to pathogenic bacteria that can cause many different infections such as, for example, ear infections, eye infections, and sinusitis. Many different strains of Haemophilus influenzae have been isolated and have an IhfA gene or protein. Some non-limiting examples of different strains of Haemophilus influenzae include Rd KW20, 86-028NP, R2866, PittGG, PittEE, R2846, and 2019.

“Microbial DNA” intends single or double stranded DNA from a microorganism that produces a biofilm.

As used herein, the term “label” or “detectable label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide of this disclosure can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein the term “eDNA” refers to extracellular DNA found as a component to pathogenic biofilms.

As used herein, the ESKAPE pathogens include Enterococcus faecium, Staphylococcus aureus, Klebsiella pneumoniae, Acinetobacter baumannii, Pseudomonas aeruginosa, and Enterobacter species. These pathogens are the leading cause of nosocomial infections throughout the world.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are known in the art. Such vectors are capable of transcribing RNA in vitro or in vivo, and are commercially available from sources such as Stratagene (La Jolla, CA) and Promega Biotech (Madison, WI). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this disclosure. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this disclosure are other non-limiting techniques.

“Inhibiting, preventing or breaking down” a biofilm intends the prophylactic or therapeutic reduction in the structure of a biofilm. In one aspect, the definition excludes prevention of a biofilm. In another aspect, the terms “inhibiting, competing or titrating” intend a reduction in the formation of the DNA/protein matrix that is a component of a microbial biofilm. In one aspect, prevention is excluded from treatment.

A “bent polynucleotide” intends a double strand polynucleotide that contains a small loop on one strand which does not pair with the other strand and any polynucleotide where the end to end distance is reduced beyond natural thermal fluctuations i.e. that is bending beyond the persistence length of 150 bp for native B-form double stranded DNA. In some embodiments, the loop is from 1 base to about 20 bases long, or alternatively from 2 bases to about 15 bases long, or alternatively from about 3 bases to about 12 bases long, or alternatively from about 4 bases to about 10 bases long, or alternatively has about 4, 5, or 6, or 7, or 8, or 9 or 10 bases.

A “subject” of diagnosis or treatment is a cell or an animal such as a mammal or a human. Non-human animals subject to diagnosis or treatment and are those subject to infections or animal models, for example, simians, murines, such as, rats, mice, chinchilla, canine, such as dogs, leporids, such as rabbits, livestock, sport animals and pets.

The term “protein”, “peptide” and “polypeptide” are used interchangeably and in their broadest sense refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart.

It is to be inferred without explicit recitation and unless otherwise intended, that when the present disclosure relates to a polypeptide, protein, polynucleotide, or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or alternatively about 80% homology or identity and alternatively, at least about 85%, or alternatively at least about 90%, or alternatively at least about 95% or alternatively 98% percent homology or identity and exhibits substantially equivalent biological activity to the reference protein, polypeptide or nucleic acid. In another aspect, the term intends a polynucleotide that hybridizes under conditions of high stringency to the reference polynucleotide or its complement.

A polynucleotide or polynucleotide region (or a polypeptide or polypeptide region) having a certain percentage (for example, 80%, 85%, 90% or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 30% identity or alternatively less than 25% identity, less than 20% identity, or alternatively less than 10% identity with one of the sequences of the present disclosure.

“Homology” or “identity” or “similarity” can also refer to two nucleic acid molecules that hybridize under stringent conditions to the reference polynucleotide or its complement.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets. The term “subject,” “host,” “individual,” and “patient” are as used interchangeably herein to refer to animals, typically mammalian animals. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments a subject is a human.

A host cell intends a eukaryotic or prokaryotic cell comprising an exogenous agent. “Eukaryotic cells” comprise all of the life kingdoms except Monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, the terms “treating,” “treatment” and the like are used herein to mean obtaining a desired pharmacologic and/or physiologic effect. The effect may be prophylactic in terms of completely or partially preventing a disorder or sign or symptom thereof and/or may be therapeutic in terms of a partial or complete cure for a disorder and/or adverse effect attributable to the disorder. In one aspect, “treatment” excludes prevention.

To “prevent” intends to prevent a disorder or effect in vitro or in vivo in a system or subject that is predisposed to the disorder or effect. An example of such is preventing the formation of a biofilm in a system that is infected with a microorganism known to produce one.

“Pharmaceutically acceptable carriers” refers to any diluents, excipients or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like and consistent with conventional pharmaceutical practices.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include oral administration, nasal administration, injection and topical application.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “effective amount” refers to a quantity sufficient to achieve a beneficial or desired result or effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

The agents and compositions can be used in the manufacture of medicaments and for the treatment of humans and other animals by administration in accordance with conventional procedures, such as an active ingredient in pharmaceutical compositions.

An agent of the present disclosure can be administered for therapy by any suitable route of administration. It will also be appreciated that the preferred route will vary with the condition and age of the recipient and the disease being treated.

An example of a solid phase support include glass, polystyrene, polypropylene, polyethylene, dextran, nylon, amylases, natural and modified celluloses, polyacrylamides, gabbros and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead or cylindrical, as in the inside surface of a test tube or the external surface of a rod. Alternatively, the surface may be flat such as a sheet, test strip, etc. or alternatively polystyrene beads. Those skilled in the art will know many other suitable carriers for binding antibody or antigen or will be able to ascertain the same by use of routine experimentation.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region or any portion thereof or at least one portion of a binding protein. The antibodies can be polyclonal or monoclonal and can be isolated from any suitable biological source, e.g., murine, rat, sheep or canine.

The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; and multispecific antibody fragments formed from antibody fragments. Examples of such include, but are not limited to a complementarity determining region (CDR) of a heavy or light chain or a ligand binding portion thereof, a heavy chain or light chain variable region (which is also referred to herein as a variable domain), a heavy chain or light chain constant region (which is also referred to herein as a constant domain), a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The term “anti-” when used before a protein name, anti-DNABII, anti-IHF, anti-HU, anti-tip chimer, for example, refers to a monoclonal or polyclonal antibody that binds and/or has an affinity to a particular protein. For example, “anti-IHF” refers to an antibody that binds to the IHF protein. The specific antibody may have affinity or bind to proteins other than the protein it was raised against. For example, anti-IHF, while specifically raised against the IHF protein, may also bind other proteins that are related either through sequence homology or through structure homology.

Complementarity determining regions (CDRs) are part of the variable region of an antibody or a T cell receptor generated by B-cell s and T-cells respectively, wherein these molecules bind to their specific antigen (also called epitope). In certain embodiments, the terms “variable region” and “variable domain” are used interchangeably, referring to the polypeptide of a light or heavy chain of an antibody that varies greatly in its sequence of amino acid residues from one antibody to another, and that determines the conformation of the combining site which confers the specificity of the antibody for a particular antigen. In a further embodiment, the variable region is about 90 amino acids long to about 200 amino acids long, including but not limited to about 100 amino acids long, or alternatively about 110 amino acids long, or alternatively about 120 amino acids long, or alternatively about 130 amino acids long, or alternatively about 140 amino acids long, or alternatively about 150 amino acids long, or alternatively about 160 amino acids long, or alternatively about 170 amino acids long, or alternatively about 180 amino acids long, or alternatively about 190 amino acids long. In certain embodiments, a variable region of an amino acid sequence, as used herein, refers to that the first about 100 amino acids, or alternatively about 110 amino acids, or alternatively about 120 amino acids, or alternatively about 130 amino acids, or alternatively about 140 amino acids, or alternatively about 150 amino acids of the amino acid sequence (including or excluding a signal peptide if applicable) is the variable region.

A set of CDRs constitutes a paratope also called an antigen-binding site, which is a part of an antibody that recognizes and binds to an antigen. There are three CDRs (CDR1, CDR2 and CDR3), arranged non-consecutively, optionally from the amino terminus to the carboxyl terminus, on the amino acid sequence of a variable region of an antigen receptor, such as a heavy chain or a light chain. As used herein, CDRn refers to a CDRn in an immunoglobulin chain or derived from an immunoglobulin chain, wherein the number n is selected from 1-3. In one embodiment, CDRLn refers to a CDRn in a light chain or derived from a light chain, wherein the number n is selected from 1-3; while CDRHn refers to a CDRn in a heavy chain or derived from a heavy chain, wherein the number n is selected from 1-3. In certain embodiments, framework region (FR) refers to the part of a variable region which is not a CDR. In certain embodiments, FRn refers to a FR in a heavy chain or a light chain or derived from a heavy chain or a light chain, and wherein the number n is selected from 1-4. In certain embodiments, a variable region comprises or consists essentially of, or yet further consists of the following (optionally following the order as provided, and further optionally from the amino terminus to the carboxyl terminus): FR1, CDR1, FR2, CDR2, FR3, CDR3 and FR4.

Variable regions and/or CDRs of an antibody or a fragment thereof can be determined by one of skill in the art, for example, using publicly or commercially available tools. Non-limiting examples of such tools include, IgBlast (accessible at www.ncbi.nlm.nih.gov/igblast/), Scaligner (available from drugdesigntech at www.scaligner.com/), IMGT rules and/or tools (see, for example, www.imgt.org/IMGTScientincChart/Nomenclature/IMGT-FRCDRdefinition.html, also accessible at www.imgt.org/), Chothia Canonical Assignment (accessible at www.bioinf.org.uk/abs/chothia.html), Antigen receptor Numbering And Receptor Classification (ANARCI, accessible at opig.stats.ox.ac.uk/webapps/newsabdab/sabpred/anarci/), the Kabat numbering method/scheme (e.g., Kabat, E. A., et al., (1991) Sequences of Proteins of Immunological Interest, Fifth Edition, U.S. Department of Health and Human Services, NIH Publication No. 91-3242,) or the Paratome web server (accessible at www.ofranlab.org/paratome/, see Vered Kunik, et al, Nucleic Acids Research, Volume 40, Issue W1, 1 Jul. 2012, Pages W521-W524).

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous sample of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region or a fragment thereof (for example, 1, 2, 3, 4, 5, or all 6 CDRs) of the recipient are replaced by residues from a variable region or a fragment thereof (for example, 1, 2, 3, 4, 5, or all 6 CDRs) of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. Without wishing to be bound by the theory, humanized antibodies produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine. Specifically, the humanized antibodies as disclosed herein specifically binds to a DNABII polypeptide or a fragment thereof (such as the tip chimeric peptide or the tail chimeric peptide) with certain range(s) of one or more of the following: EC₅₀, Kₒₙ, K_{off}, K_{A} and/or K_{D}, and inhibits or releases certain cytokine(s) upon treating a subject. In a further embodiment, the humanized antibody specifically binding to the tip region of a DNABII polypeptide (such as the tip chimeric peptide) disrupts biofilm both in vivo and in vitro. In addition, the process of humanization, while a rational design process, may produce unexpected changes (positive or negative) in, e.g., binding affinity, antigen specificity, or physical properties such as solubility or aggregability; hence, properties of humanized antibodies are not inherently predictable from the properties of the starting non-human antibody.

In one embodiment, an antibody as used herein may be a recombinant antibody. The term “recombinant antibody”, as used herein, includes all antibodies that are prepared, expressed, created or isolated by recombinant means, such as antibodies isolated from an animal (e.g., a mouse) that is transgenic or transchromosomal for immunoglobulin genes or a hybridoma prepared therefrom, antibodies isolated from a host cell transformed to express the antibody, e.g., from a transfectoma, antibodies isolated from a recombinant, combinatorial antibody library, and antibodies prepared, expressed, created or isolated by any other means that involve splicing of immunoglobulin (Ig) gene sequences to other DNA sequences. In certain embodiments, however, such recombinant antibodies can be subjected to in vitro mutagenesis (or, when an animal transgenic for Ig sequences is used, in vivo somatic mutagenesis) and thus the amino acid sequences of the VH and VL regions of the recombinant antibodies are sequences that may not naturally exist within the antibody germline repertoire in vivo. Methods to making these antibodies are known in the art.

In one embodiment, an antibody as used herein may be a chimeric antibody. As used herein, chimeric antibodies are antibodies whose light and heavy chain genes have been constructed, typically by genetic engineering, from antibody variable and constant region genes belonging to different species.

In addition, the antibodies disclosed herein may be engineered to include modifications within the Fc region to alter one or more functional properties of the antibody, such as serum half-fife, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (U.S. Pat. No. 5,677,425) and amino acid mutations in the Fc hinge region to decrease the biological half-life of the antibody (U.S. Pat. No. 6,165,745).

Additionally, the antibodies disclosed herein may be chemically modified. Glycosylation of an antibody can be altered, for example, by modifying one or more sites of glycosylation within the antibody sequence to increase the affinity of the antibody for antigen (U.S. Pat. Nos. 5,714,350 and 6,350,861). Alternatively, to increase antibody-dependent cell-mediated cytotoxicity, a hypofucosylated antibody having reduced amounts of fucosyl residues or an antibody having increased bisecting GlcNac structures can be obtained by expressing the antibody in a host cell with altered glycosylation mechanism (Shields, R. L. et al. (2002) J. Biol. Chem. 277:26733-26740; Umana et al. (1999) Nat. Biotech. 17:176-180).

The antibodies disclosed herein can be pegylated to increase biological half-life by reacting the antibody or fragment thereof with polyethylene glycol (PEG) or a reactive ester or aldehyde derivative of PEG, under conditions in which one or more PEG groups become attached to the antibody or antibody fragment. Antibody pegylation may be carried out by an acylation reaction or an alkylation reaction with a reactive PEG molecule (or an analogous reactive water soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide.

The antibody to be pegylated can be an aglycosylated antibody. Methods for pegylating proteins are known in the art and can be applied to the antibodies disclosed herein (EP 0154316 and EP 0401384).

Additionally, antibodies may be chemically modified by conjugating or fusing the antigen-binding region of the antibody to serum protein, such as human serum albumin, to increase half-life of the resulting molecule. Such approach is for example described in EP 0322094 and EP 0486525.

The antibodies or fragments thereof of the present disclosure may be conjugated to a diagnostic agent and used diagnostically, for example, to monitor the development or progression of a disease and determine the efficacy of a given treatment regimen. Examples of diagnostic agents include enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or fragment thereof, or indirectly, through a linker using techniques known in the art. Examples of suitable enzymes include horseradish peroxidase, alkaline phosphatase, beta-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin. Examples of suitable radioactive material include ¹²⁵I,¹³¹I, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-1105, Palladium-109, Praseodymium-143, Promethium-149, Erbium-169, Iridium-194, Gold-198, Gold-199, and Lead-211. Monoclonal antibodies may be indirectly conjugated with radiometal ions through the use of bifunctional chelating agents that are covalently linked to the antibodies. Chelating agents may be attached through amities (Meares et al. (1984) Anal. Biochem. 142:68-78); sulfhydral groups (Koyama (1994) Chem. Abstr. 120:217-262) of amino acid residues and carbohydrate groups (Rodwell et al. (1986) PNAS USA 83:2632-2636; Quadri et al. (1993) Nucl. Med. Biol. 20:559-570).

Further, the antibodies or fragments thereof of the present disclosure may be conjugated to a therapeutic agent. Suitable therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabine, cladribine), alkylating agents (such as mechlorethamine, thioepa, chloramhucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), diphtheria toxin and related molecules (such as diphtheria A chain and active fragments thereof and hybrid molecules), ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, gelonin, mitogellin, restrictocin, phenomycin, enomycin toxins and mixed toxins.

Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. Aptamers are small nucleic acids ranging from 15-50 bases in length that fold into defined secondary and tertiary structures, such as stem-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that are capable of catalyzing a chemical reaction, either intramolecularly or intermolecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity.

The functional nucleic acid molecules may act as effectors, inhibitors, modulators, and stimulators of a specific activity possessed by a target molecule, or the functional nucleic acid molecules may possess a de novo activity independent of any other molecules.

The therapeutic agents can be linked to the antibody directly or indirectly, using any of a large number of available methods. For example, an agent can be attached at the hinge region of the reduced antibody component via disulfide bond formation, using cross-linkers such as N-succinyl 3-(2-pyridyldithio)propionate (SPDP), or via a carbohydrate moiety in the Fc region of the antibody (Yu et al. 1994 Int. J. Cancer 56: 244; Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, engineering and clinical application, Ritter et al. (eds.), pages 60-84 (Cambridge University Press 1995)).

Techniques for conjugating therapeutic agents to antibodies are well known (Amon et al. “Monoclonal Antibodies For Immunotargeting Of Drugs In Cancer Therapy,” in Monoclonal Antibodies And Cancer Therapy; Reisfeld et al. (eds.), pp. 243-56 (Alan R. Liss, Inc. 1985); Hellstrom et al. “Antibodies For Drug Delivery,” in Controlled Drug Delivery (2nd Ed.); Robinson et al. (eds.), pp. 623-53 (Marcel Dekker, Inc. 1987); Thorpe “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody in Cancer Therapy,” in Monoclonal Antibodies For Cancer Detection And Therapy, Baldwin et al. (eds.), pp. 303-16 (Academic Press 1985), and Thorpe et al. “The Preparation And Cytotoxic Properties Of Antibody-Toxin Conjugates,” (1982) Immunol. Rev. 62:119-58).

The antibodies disclosed herein or antigen-binding regions thereof can be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide or binding mimetic, can be done, for example, by chemical coupling, genetic fusion, or noncovalent association. Multi-specific molecules can further include a third binding specificity, in addition to the first and second target epitope.

Bi-specific and multi-specific molecules can be prepared using methods known in the art. For example, each binding unit of the hi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitroberizoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohexane-I-carboxylate (sulfo-SMCC) (Karpovsky et al. (1984) J. Exp. Med. 160:1686; Liu et al. (1985) Proc. Natl. Acad. Sci. USA 82:8648). When the binding molecules are antibodies, they can be conjugated by sulfhydryl bonding of the C-terminus hinge regions of the two heavy chains.

The antibodies or fragments thereof of the present disclosure may be linked to a moiety that is toxic to a cell to which the antibody is bound to form “depleting” antibodies. These antibodies are particularly useful in applications where it is desired to deplete an NK cell.

The antibodies disclosed herein may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibodies also can be bound to many different carriers. Thus, this disclosure also provides compositions containing the antibodies and another substance, active or inert.

Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble for purposes disclosed herein. Those skilled in the art will know of other suitable carriers for binding monoclonal antibodies, or will be able to ascertain such, using routine experimentation.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

“Immune response” broadly refers to the antigen-specific responses of lymphocytes to foreign substances. Any substance that can elicit an immune response is said to be “immunogenic” and is referred to as an “immunogen”. All immunogens are antigens, however, not all antigens are immunogenic. An immune response of this disclosure can be humoral (via antibody activity) or cell-mediated (via T cell activation).

The term “modulate an immune response” includes inducing (increasing, eliciting) an immune response; and reducing (suppressing) an immune response. An immunomodulatory method (or protocol) is one that modulates an immune response in a subject.

An “HMG domain” or “high mobility group (HMG) box domain” refers to an amino acid sequence that is involved in binding DNA (Stros et al., Cell Mol Life Sci. 64(19-20):2590-606 (2007)). In one embodiment, the structure of the HMG-box domain consists of three helices in an irregular array. In another embodiment, an HMG-box domain enables a protein to bind non-B-type DNA conformations (kinked or unwound) with high affinity. HMG-box domains can be found in high mobility group proteins, which are involved in the regulation of DNA-dependent processes such as transcription, replication and DNA repair, all of which require changing the conformation of chromatin (Thomas (2001) Biochem. Soc. Trans. 29(Pt 4):395-401).

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, a “recombinant peptide” refers to a peptide produced in a host cell (e.g., E. coli, yeast) using exogenous/recombinant nucleic acids. As used herein, a “synthetic peptide” refers to a peptide synthesized by synthetic or chemical means, without using a host cell. While a recombinant peptide may have post-translational modifications, a synthetic peptide does not have any post-translational modifications.

In certain embodiments, the biofilm is derived from (i.e., produced by) a gram negative or a gram-positive biofilm producing bacteria. In certain embodiments, the biofilm comprises a DNABII protein. In a further embodiment, the biofilm comprises a histone-like protein from E. coli strain U93 (HU) or an integration host factor (IHF) binding protein. In certain embodiments, the DNABII peptide is an IHF peptide. Additionally or alternatively, the DNABII peptide is an HU peptide. In certain embodiments, the tip region of DNABII peptide is the tip region of IHFA and/or the tip region of IHFB. In a further embodiment, the tip region of DNABII peptide is an IHFA tip region conjugated directly or indirectly (for example via a linker) to an IHFB tip region. In yet a further embodiment, the tip region of DNABII peptide is the IhfA5-mIhfB4_{NTHI} tip chimeric peptide.

As used herein, the term “EC₅₀” refers to the concentration of an antibody or a fragment thereof which induces a response (for example, binding between the antibody or a fragment thereof and its target) halfway between the baseline and maximum after a specified exposure time.

Several parameters are used herein to describe the binding and unbinding reaction of receptor (R, such as an antibody or a fragment thereof) and ligand (L, such as the target of the antibody or a fragment thereof) molecules, which is formalized as: R+L⇄RL. The reaction is characterized by the on-rate constant kₒₙ and the off-rate constant k_{off}, which have units of M⁻¹ s⁻¹ and s⁻¹, respectively. In equilibrium, the forward binding transition R+L→RL should be balanced by the backward unbinding transition RL→R+L. That is kₒₙ [R] [L]=k_{off} [RL] where [R], [L] and [RL] represent the concentration of unbound free receptors, the concentration of unbound free ligand and the concentration of receptor-ligand complexes. Further, the equilibrium dissociation constant “K_{D}” can be calculated as k_{off}/kₒₙ which is [R]×[L]/[RL], while the equilibrium association constant “K_{A}” can be calculated as kₒₙ/k_{off} which is [RL]/([R]×[L]).

### MODES FOR CARRYING OUT THE DISCLOSURE

### Synthetic or Recombinant mB Box-97 Polypeptide

Provided herein is a synthetic or recombinant polypeptide comprising, or consisting essentially of, or consisting of mB Box-97 that consists of the amino acids 90 to 176 or amino acids 80 to 176 from the coding sequence of the native human HMGB1 protein (as shown in SEQ ID NO: 2) with a cysteine to serine point mutation at amino acid 106 that abrogates the ability of this polypeptide to induce an inflammatory response as well as equivalents thereof that retain the cysteine to serine point mutation at amino acid 106 mB Box-97, consists of the amino acids 90 to 176 or amino acids 80 to 176 from the coding sequence of the native human HMGB1 protein (native/wild type human HMGB1 sequence is shown in SEQ ID NO: 2) that abrogate the ability of this polypeptide to induce an inflammatory response. The synthetic polypeptide is produced by synthetic or chemical means and is not wild type or produced recombinantly.

As used herein, an equivalent of a mB Box-97 polypeptide, a recombinant mB Box-97, or synthetic mB Box-97 refers to a sequence that is at least about 70%, or alternatively at least about 75%, or at least about 80%, or at least about 85%, or at least about 90%, or at least about 95%, or at least about 98% or at least about 99% identical to the reference mB Box-97 polypeptide a recombinant mB Box-97, or synthetic mB Box-97 that in one aspect, retain the mutated amino acid of a cysteine to serine point mutation at amino acid 106. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

In some respects, the equivalent of an mB Box-97 polypeptide retains the intended function and/or structural characteristics of the mB Box-97polypeptide. In another aspect, the equivalent of mB-Box-97 polypeptide includes the mB Box-97, or the recombinant mB Box-97, or the synthetic mB Box-97 polypeptides that retain the C to S amino acid substitution at amino acid 106, and which further contain independently at least 2, or alternatively at least 3, or alternatively at least 4, or alternatively at least 5, or at least 6, or alternatively at least 7, or alternatively at least 8, or alternatively at least 9 or alternatively at least 10 amino acids at the amino and/or carboxyl terminus of the polypeptide.

In a further aspect, the mB Box-97 HMGB1 polypeptide further comprises or consists essentially of, or yet further consists of one or more linker polypeptides. An example of a peptide linker is GPSLKL (SEQ ID NO: 3) or PPKGETKKKF (SEQ ID NO: 4) on the amine and/or carboxy terminus.

The polypeptides can be detectably labeled and/or combined with a carrier, e.g., a pharmaceutically acceptable carrier.

The disclosed mB Box-97 HMGB1 polypeptide is used to treat or prevent aberrant or excessive NET formation in a subject in need thereof by administering an effective amount of the mB Box-97 HMGB1 polypeptide to the subject. In one aspect, the subject is suffering from one or more of: a SARS CoV-2 infection, sepsis, autoimmune diseases e.g., Systemic Lupus Erythematosus, Rheumatoid arthritis, Type I diabetes mellitus, small vessel vasculitis, autoinflammatory diseases e.g., Gout, Inflammatory Bowel Disease, and metabolic diseases e.g. Type 2 diabetes and obesity. Also provided are methods to prevent NET-mediated disease or prevent the progression of NET-mediated disease in a subject in need thereof by administering an effective amount of the mB Box-97 HMGB1 polypeptide to the subject. Further provided are methods to prevent and treat bacterial biofilms by administering an effective amount of the mB Box-97 HMGB1 polypeptide to the subject.

### Synthetic or Recombinant mB Box-97 Polypeptide and Compositions Comprising the Same

An aspect of the disclosure is directed to a synthetic polypeptide comprising, or consisting essentially of, or yet further consisting of mB Box-97 that consists of the amino acids 90 to 176 from the coding sequence of the native human HMGB1 protein shown as SEQ ID NO: 2 with a cysteine to serine point mutation at amino acid 106 or an equivalent thereof.

Another aspect of the disclosure is directed to a recombinant polypeptide comprising, or consisting essentially of, or yet further consisting of mB Box-97 that consists of the amino acids 90 to 176 from the coding sequence of the native human HMGB1 protein shown as SEQ ID NO: 2 with a cysteine to serine point mutation at amino acid 106 or an equivalent thereof.

As used herein, an equivalent of the recombinant mB Box-97 or the synthetic mB Box-97 refers to a sequence that is at least about 70%, or alternatively at least about 75%, or at least about 80%, or at least about 85%, or at least about 90%, or at least about 95%, or at least about 98% or at least about 99% identical to the reference recombinant mB Box-97, or the reference synthetic mB Box-97 that in one aspect, retain the mutated amino acid of a cysteine to serine point mutation at amino acid 106. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

In some respects, the equivalent of the recombinant polypeptide retains the intended function and/or structural characteristics of the mB Box-97polypeptide. In another aspect, the equivalent of mB-Box-97 includes the recombinant mB Box-97 polypeptide that retains the C to S amino acid substitution at amino acid 106, and which further contain independently at least 2, or alternatively at least 3, or alternatively at least 4, or alternatively at least 5, or at least 6, or alternatively at least 7, or alternatively at least 8, or alternatively at least 9 or alternatively at least 10 amino acids at the amino and/or carboxyl terminus of the polypeptide.

In a further aspect, the recombinant mB Box-97 HMGB1 polypeptide further comprises or consists essentially of, or yet further consists of one or more linker polypeptides. An example of a peptide linker is GPSLKL (SEQ ID NO: 3) or PPKGETKKKF (SEQ ID NO: 4) on the amine and/or carboxy terminus.

In some embodiments, the recombinant polypeptide consists of SEQ ID NO: 5.

Another aspect of the disclosure is directed to a synthetic polypeptide comprising mB Box-97 that consists of the amino acids 80 to 176 from the coding sequence of the native human HMGB1 protein shown as SEQ ID NO: 2 with a cysteine to serine point mutation at amino acid 106 or an equivalent thereof.

As used herein, an equivalent of the synthetic mB Box-97 refers to a sequence that is at least about 70%, or alternatively at least about 75%, or at least about 80%, or at least about 85%, or at least about 90%, or at least about 95%, or at least about 98% or at least about 99% identical to the reference synthetic mB Box-97 that in one aspect, retain the mutated amino acid of a cysteine to serine point mutation at amino acid 106. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

In some respects, the equivalent of a synthetic mB Box-97 polypeptide retains the intended function and/or structural characteristics of the synthetic mB Box-97polypeptide. In another aspect, the equivalent of the synthetic mB-Box-97 that retains the C to S amino acid substitution at amino acid 106, and which further contain independently at least 2, or alternatively at least 3, or alternatively at least 4, or alternatively at least 5, or at least 6, or alternatively at least 7, or alternatively at least 8, or alternatively at least 9 or alternatively at least 10 amino acids at the amino and/or carboxyl terminus of the polypeptide.

In a further aspect, the synthetic mB Box-97 HMGB1 polypeptide or an equivalent thereof further comprises or consists essentially of, or yet further consists of one or more linker polypeptides. An example of a peptide linker is GPSLKL (SEQ ID NO: 3) or PPKGETKKKF (SEQ ID NO: 4) on the amine and/or carboxy terminus.

Another aspect of the disclosure is directed to a recombinant or synthetic polypeptide comprising, or consisting essentially of, or yet further consisting of the recombinant or synthetic mB Box-97 that consists of the amino acids 80 to 176 from the coding sequence of the native human HMGB1 protein shown as SEQ ID NO: 2 with a cysteine to serine point mutation at amino acid 106 or an equivalent thereof.

In some embodiments, the synthetic or recombinant polypeptide consists of SEQ ID NO: 6.

In some embodiments, an equivalent comprises an amino acid sequence having at least about 80% homology or amino acid identity thereto, or an amino acid encoded by polynucleotide that hybridizes under conditions of high stringency to a polynucleotide encoding the amino acid sequence or its complement, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water; and wherein the equivalent of the cysteine to serine point mutation at amino acid 106 of SEQ ID NO: 2 is a serine to alanine mutation at amino acid 106 of SEQ ID NO: 2.

In some embodiments, the synthetic or recombinant polypeptide comprises a detectable label.

In some embodiments, the synthetic or recombinant polypeptide or the equivalent of each thereof comprises a linker polypeptide, optionally wherein the linker polypeptide comprises GPSLKL (SEQ ID NO: 3) or PPKGETKKKF (SEQ ID NO: 4). In some embodiments, the linker is at the C terminal of the synthetic or recombinant polypeptide. In some embodiments, the linker is at the N terminal of the synthetic or recombinant polypeptide.

Another aspect of the disclosure is directed to a plurality of the synthetic or recombinant polypeptide of present disclosure. In some embodiments, the members of the plurality are the same or different from each other.

Another aspect of the disclosure is directed to a composition comprising, or consisting essentially of, or yet further consisting of the synthetic or recombinant polypeptide of the present disclosure or the plurality of the present disclosure, and a carrier. In some embodiments, the carrier is a pharmaceutically acceptable carrier.

The isolated polypeptides disclosed herein are intended to include recombinantly produced polypeptides and proteins from prokaryotic and eukaryotic host cells, as well as muteins, analogs and fragments thereof, examples of such cells are described above.

It is understood that functional equivalents or variants of the wild type polypeptide or protein also are within the scope of this disclosure, for example, those having conservative amino acid substitutions of the amino acids.

In a further aspect, the polypeptides are conjugated or linked to a detectable label or an agent to increase the half-life of the polypeptide, e.g., PEGylation a PEG mimetic, polysialylation, HESylation or glycosylation. Suitable labels are known in the art and described herein.

The proteins and polypeptides are obtainable by a number of processes known to those of skill in the art, which include purification, chemical synthesis and recombinant methods. Accordingly, this disclosure also provides the methods to produce the mB Box-97 polypeptides using these methods and the polynucleotides and polypeptides as disclosed herein and the methods, also disclosed herein. For example, for recombinant mB Box-97 can be produced in host cell systems comprising a polynucleotide encoding the polypeptide and culturing the host cell under conditions that favor the recombinant production of the polypeptides. Polypeptides can be isolated from the host cell systems by methods such as immunoprecipitation with antibody, and standard techniques such as gel filtration, ion-exchange, reversed-phase, and affinity chromatography. For such methodology, see for example Deutscher et al. (1999) Guide To Protein Purification: Methods In Enzymology (Vol. 182, Academic Press). Accordingly, this disclosure also provides the processes for obtaining these polypeptides as well as the products obtainable and obtained by these processes.

The polypeptides also can be obtained by chemical synthesis using a commercially available automated peptide synthesizer such as those manufactured by Perkin/Elmer/Applied Biosystems, Inc., Model 430A or 431A, Foster City, Calif., USA. The synthesized polypeptide can be precipitated and further purified, for example by high performance liquid chromatography (HPLC). Accordingly, this disclosure also provides a process for chemically synthesizing the proteins disclosed herein by providing the sequence of the protein and reagents, such as amino acids and enzymes and linking together the amino acids in the proper orientation and linear sequence.

Also provided by this disclosure are the polypeptides described herein conjugated to a detectable agent for use in the diagnostic methods. For example, detectably labeled polypeptides can be bound to a column and used for the detection and purification of antibodies. They also are useful as immunogens for the production of antibodies. The polypeptides disclosed herein are useful in an in vitro assay system to screen for agents or drugs, which modulate cellular processes.

It is well known to those skilled in the art that modifications can be made to the peptides disclosed herein to provide them with altered properties. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

Peptides disclosed herein can be modified to include unnatural amino acids. Thus, the peptides may comprise D-amino acids, a combination of and L-amino acids, and various “designer” amino acids (e.g., beta-methyl amino acids, C-alpha-methyl amino acids, and N-alpha-methyl amino acids, etc.) to convey special properties to peptides. Additionally, by assigning specific amino acids at specific coupling steps, peptides with alpha-helices, beta. turns, beta. sheets, gamma-turns, and cyclic peptides can be generated. Generally, it is believed that alpha-helical secondary structure or random secondary structure may be of particular use.

The polypeptides disclosed herein also can be combined with various solid phase carriers, such as an implant, a stent, a paste, a gel, a dental implant, or a medical implant or liquid phase carriers, such as beads, sterile or aqueous solutions, pharmaceutically acceptable carriers, pharmaceutically acceptable polymers, liposomes, micelles, suspensions and emulsions. Examples of non-aqueous solvents include propyl ethylene glycol, polyethylene glycol and vegetable oils. When used to prepare antibodies or induce an immune response in vivo, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides. Other suitable adjuvants include monophosphoryl lipid A (MPL), mutant derivatives of the heat labile enterotoxin of E. coli, mutant derivatives of cholera toxin, CPG oligonucleotides, and adjuvants derived from squalene.

This disclosure also provides a pharmaceutical composition comprising or alternatively consisting essentially of, or yet further consisting of, any of a polypeptide, analog, mutein, or fragment disclosed herein, alone or in combination with each other or other agents, such an antibiotic and an acceptable carrier or solid support or an antibody or fragment thereof as described herein. These compositions are useful for various diagnostic and therapeutic methods as described herein.

### Isolated Polynucleotides, Vectors, Isolated Host Cells

Another aspect of the disclosure is directed to an isolated polynucleotide encoding the synthetic or recombinant polypeptide of the present disclosure for example a synthetic or recombinant polypeptide comprising, or consisting essentially of, or consisting of mB Box-97 that consists of the amino acids 80 to 176 from the coding sequence of the native human HMGB1 protein with a cysteine to serine point mutation at amino acid 106 that abrogates the ability of this polypeptide to induce an inflammatory response as well as equivalents thereof that retain the cysteine to serine point mutation at amino acid 106 mB Box-97, and consists of the amino acids 80 to 176 from the coding sequence of the native human HMGB1 protein (native/wild type human HMGB1 sequence is shown in SEQ ID NO: 2) that abrogate the ability of this polypeptide to induce an inflammatory response. Also provided are complementary polynucleotides to the polynucleotides encoding the polypeptides. The polynucleotides can be DNA, RNA, mRNA or interfering RNA, such as siRNA, miRNA or dsRNA.

The disclosure also provides a polynucleotide encoding an equivalent of a mB Box-97 polypeptide, a recombinant mB Box-97, or synthetic mB Box-97 refers to a sequence that is at least about 70%, or alternatively at least about 75%, or at least about 80%, or at least about 85%, or at least about 90%, or at least about 95%, or at least about 98% or at least about 99% identical to the reference mB Box-97 polypeptide a recombinant mB Box-97, or synthetic mB Box-97 that in one aspect, retain the mutated amino acid of a cysteine to serine point mutation at amino acid 106. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The polynucleotides can be DNA, RNA, mRNA or interfering RNA, such as siRNA, miRNA or dsRNA.

In some respects, the polynucleotide encodes an equivalent of a polypeptide that retains the intended function and/or structural characteristics of the synthetic or recombinant mB Box-97polypeptide. In another aspect, the equivalent of the synthetic or recombinant mB-Box-97 includes the recombinant mB Box-97, or the synthetic mB Box-97 polypeptides that retain the C to S amino acid substitution at amino acid 106, and which further contain independently at least 2, or alternatively at least 3, or alternatively at least 4, or alternatively at least 5, or at least 6, or alternatively at least 7, or alternatively at least 8, or alternatively at least 9 or alternatively at least 10 amino acids at the amino and/or carboxyl terminus of the polypeptide. The polynucleotides can be DNA, RNA, mRNA or interfering RNA, such as siRNA, miRNA or dsRNA.

In a further aspect, the polynucleotide encodes a mB Box-97 HMGB1 polypeptide that further comprises or consists essentially of, or yet further consists of one or more linker polypeptides. An example of a peptide linker is GPSLKL (SEQ ID NO: 3) or PPKGETKKKF (SEQ ID NO: 4) on the amine and/or carboxy terminus. The polynucleotides can be DNA, RNA, mRNA or interfering RNA, such as siRNA, miRNA or dsRNA.

The polynucleotides can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In one aspect, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates can be employed to provide a means visible to the human eye or spectrophotometrically, to identify specific hybridization with complementary nucleic acid-containing samples. Thus, this disclosure further provides a method for detecting a single-stranded polynucleotide or its complement, by contacting target single-stranded polynucleotide with a labeled, single-stranded polynucleotide (a probe) which is a portion of the polynucleotide disclosed herein under conditions permitting hybridization (optionally moderately stringent hybridization conditions) of complementary single-stranded polynucleotides, or optionally, under highly stringent hybridization conditions. Hybridized polynucleotide pairs are separated from un-hybridized, single-stranded polynucleotides. The hybridized polynucleotide pairs are detected using methods known to those of skill in the art and set forth, for example, in Sambrook et al. (1989) supra.

The polynucleotide embodied in this disclosure can be obtained using chemical synthesis, recombinant cloning methods, PCR, or any combination thereof. Methods of chemical polynucleotide synthesis are known in the art and need not be described in detail herein. One of skill in the art can use the sequence data provided herein to obtain a desired polynucleotide by employing a DNA synthesizer or ordering from a commercial service.

The polynucleotides disclosed herein can be isolated or replicated using PCR. The PCR technology is the subject matter of U.S. Pat. Nos. 4,683,195; 4,800,159; 4,754,065; and 4,683,202 and described in PCR: The Polymerase Chain Reaction (Mullis et al. eds., Birkhauser Press, Boston (199.4)) or MacPherson et al. (1991) and (1995) supra, and references cited therein. Alternatively, one of skill in the art can use the sequences provided herein and a commercial DNA synthesizer to replicate the DNA. Accordingly, this disclosure also provides a process for obtaining the polynucleotides disclosed herein by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the poly-nucleotide into a suitable replication vector and insert the vector into a suitable host cell (prokaryotic or eukaryotic) for replication and amplification. The DNA so amplified can be isolated from the cell by methods known to those of skill in the art. A process for obtaining polynucleotides by this method is further provided herein as well as the polynucleotides so obtained.

RNA can be obtained by first inserting a DNA polynucleotide into a suitable host cell. The DNA can be delivered by any appropriate method, e.g., by the use of an appropriate gene delivery vehicle (e.g., liposome, plasmid or vector) or by electroporation. When the cell replicates and the DNA is transcribed into RNA; the RNA can then be isolated using methods known to those of skill in the art, for example, as set forth in Sambrook et al. (1989) supra. For instance, mRNA can be isolated using various lytic enzymes or chemical solutions according to the procedures set forth in Sambrook et al. (1989) supra, or extracted by nucleic-acid-binding resins following the accompanying instructions provided by manufactures.

Polynucleotides exhibiting sequence complementarity or homology to a polynucleotide disclosed herein are useful as hybridization probes or as an equivalent of the specific polynucleotides identified herein. Since the full coding sequence of the transcript is known, any portion of this sequence or homologous sequences can be used in the methods disclosed herein.

It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. In some embodiments, a probe useful for detecting the aforementioned mRNA is at least about 80% identical to the homologous region. In some embodiments, the probe is 85% identical to the corresponding gene sequence after alignment of the homologous region; in some embodiments, it exhibits 90% identity.

These probes can be used in radioassays (e.g., Southern and Northern blot analysis) to detect, prognose, diagnose or monitor various cells or tissues containing these cells. The probes also can be attached to a solid support or an array such as a chip for use in high throughput screening assays for the detection of expression of the gene corresponding a polynucleotide disclosed herein. Accordingly, this disclosure also provides a probe comprising or corresponding to a polynucleotide disclosed herein, or its equivalent, or its complement, or a fragment thereof, attached to a solid support for use in high throughput screens.

The total size of fragment, as well as the size of the complementary stretches, will depend on the intended use or application of the particular nucleic acid segment. Smaller fragments will generally find use in hybridization embodiments, wherein the length of the complementary region may be varied, such as between at least 5 to 10 to about 100 nucleotides, or even full length according to the complementary sequences one wishes to detect.

Nucleotide probes having complementary sequences over stretches greater than 5 to 10 nucleotides in length are generally well suited, so as to increase stability and selectivity of the hybrid, and thereby improving the specificity of particular hybrid molecules obtained. In certain embodiments, one can design polynucleotides having gene-complementary stretches of 10 or more or more than 50 nucleotides in length, or even longer where desired. Such fragments may be readily prepared by, for example, directly synthesizing the fragment by chemical means, by application of nucleic acid reproduction technology, such as the PCR technology with two priming oligonucleotides as described in U.S. Pat. No. 4,603,102 or by introducing selected sequences into recombinant vectors for recombinant production. In one aspect, a probe is about 50-75 or more alternatively, 50-100, nucleotides in length.

The polynucleotides of the present disclosure can serve as primers for the detection of genes or gene transcripts that are expressed in cells described herein. In this context, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. For illustration purposes only, a primer is the same length as that identified for probes.

One method to amplify polynucleotides is PCR and kits for PCR amplification are commercially available. After amplification, the resulting DNA fragments can be detected by any appropriate method known in the art, e.g., by agarose gel electrophoresis followed by visualization with ethidium bromide staining and ultraviolet illumination.

Methods for administering an effective amount of a gene delivery vector or vehicle to a cell have been developed and are known to those skilled in the art and described herein. Methods for detecting gene expression in a cell are known in the art and include techniques such as in hybridization to DNA microarrays, in situ hybridization, PCR, RNase protection assays and Northern blot analysis. Such methods are useful to detect and quantify expression of the gene in a cell. Alternatively, expression of the encoded polypeptide can be detected by various methods. In particular, it is useful to prepare polyclonal or monoclonal antibodies that are specifically reactive with the target polypeptide. Such antibodies are useful for visualizing cells that express the polypeptide using techniques such as immunohistology, ELISA, and Western blotting. These techniques can be used to determine expression level of the expressed polynucleotide.

In some embodiments, isolated polynucleotide is in a composition with a carrier or a pharmaceutically acceptable carrier.

In some embodiments, the isolated polynucleotide comprises a detectable label, and optionally a carrier or a pharmaceutically acceptable carrier.

Also provided are a plurality of polynucleotides that can be the same or different from each other, as well as compositions containing the plurality and a carrier, such as a pharmaceutically acceptable carrier.

Another aspect of the disclosure is directed to a vector comprising, or consisting essentially of, or yet further consisting of the isolated polynucleotide of the present disclosure, and optionally a carrier or a pharmaceutically acceptable carrier. The vector can be a lipid nanoparticle, a plasmid, or a viral vector, for example.

Also provided are a plurality of vectors that can be the same or different from each other, as well as compositions containing the plurality and a carrier, such as a pharmaceutically acceptable carrier.

In some embodiments, the isolated polynucleotide or the vector further comprises a heterologous promoter sequence, and optionally a carrier or a pharmaceutically acceptable carrier.

Another aspect of the disclosure is directed to an isolated host cell containing one or more of: the synthetic or recombinant polypeptide of the present disclosure, the plurality of the present disclosure, the isolated polynucleotide of the present disclosure or the vector of the present disclosure, and optionally a carrier or a pharmaceutically acceptable carrier.

In some embodiments, the host cell is a prokaryotic or a eukaryotic cell. In some embodiments, the host cell is a eukaryotic cell. In some embodiments, the eukaryotic cell is a mammalian cell.

Further provided is a method of producing the recombinant polypeptide of this disclosure by a method comprising: delivering the isolated polynucleotide of the present disclosure or the vector of the present disclosure to a host cell under conditions where the polypeptide of this disclosure can be expressed. In some embodiments, the host cell is a bacterial cell, a yeast cell, an insect cell or a mammalian cell. In some embodiments, the host cell is an E. coli cell.

Yet further provided is a method for producing the synthetic polypeptide of this disclosure by a method comprising solid-phase peptide synthesis or solution-phase peptide synthesis.

### Compositions Comprising an Antibody or a Fragment Thereof

Another aspect of the disclosure is directed to a composition comprising an antibody or a fragment thereof, wherein the antibody or fragment thereof recognizes and binds to a tip region of a DNABII peptide, or a tip chimer (for example an IhfA5-mIhfB4_{NTHI} tip chimer as described herein). Also provided is a composition comprising one or more antibodies that recognize and bind the IhfA5-mIhfB4_{NTHI} tip chimer.

In one aspect the antibody is a monoclonal antibody, a humanized antibody, or an antigen-binding fragment thereof.

In some embodiments, the antibody or a fragment thereof that binds to a tip region of a DNABII peptide or a tip chimer, the antibody or fragment thereof comprises:
(i) a heavy chain (HC) immunoglobulin variable domain sequence comprising, or consisting essentially of, or yet further consisting of a sequence of amino acid (aa) 25 to aa 144 of SEQ ID NO: 21 or an equivalent thereof; and
(ii) a light chain (LC) immunoglobulin variable domain sequence comprising, or consisting essentially of, or yet further consisting of a sequence of aa 21 to aa 132 of SEQ ID NO: 22 or an equivalent thereof;
   or
   wherein the antibody or a fragment thereof comprises:
(i) a heavy chain (HC) immunoglobulin variable domain sequence comprising, or consisting essentially of, or yet further consisting of a sequence of aa 25 to aa 144 of SEQ ID NO: 24 or an equivalent thereof; and
(ii) a light chain (LC) immunoglobulin variable domain sequence comprising, or consisting essentially of, or yet further consisting of a sequence of aa 21 to aa 132 of SEQ ID NO: 25 or an equivalent thereof.

In some embodiments, the antibody or a fragment thereof that binds to a tip region of a DNABII peptide comprises:
a heavy chain complementarity-determining region 1 (CDRH1) comprising, or consisting essentially of, or yet further consisting of a sequence of GFTFRTY (SEQ ID NO: 47) (aa 50 to aa 56 of SEQ ID NO: 9 or 10 or 11 or 24);
a heavy chain complementarity-determining region 2 (CDRH2) comprising, or consisting essentially of, or yet further consisting of a sequence of GSDRRH (SEQ ID NO: 48) (aa 76 to aa 81 of SEQ ID NO: 9 or 10 or 11 or 24);
a heavy chain complementarity-determining region 3 (CDRH3) comprising, or consisting essentially of, or yet further consisting of a sequence of VGPYDGYYGEFDY (SEQ ID NO: 49) (aa 121 to aa 133 of SEQ ID NO: 9 or 10 or 11 or 24);
a light chain complementarity-determining region 1 (CDRL1) comprising, or consisting essentially of, or yet further consisting of a sequence of QSLLDSDGKTF (SEQ ID NO: 51) (aa 47 to aa 57 of SEQ ID NO: 15 or 16 or 17 or 25);
a light chain complementarity-determining region 2 (CDRL2) comprising, or consisting essentially of, or yet further consisting of a sequence of LVS (aa 75 to aa 77 of SEQ ID NO: 15 or 16 or 17 or 25); and
a light chain complementarity-determining region 3 (CDRL3) comprising, or consisting essentially of, or yet further consisting of a sequence of WQGTHFP (SEQ ID NO: 52) (aa 114 to aa 120 of SEQ ID NO: 15 or 16 or 17 or 25).

In some embodiments, the antibody or a fragment thereof that binds to a tip region of a DNABII peptide comprises:
a heavy chain complementarity-determining region 1 (CDRH1) comprising, or consisting essentially of, or yet further consisting of a sequence of GFTFSRYG (SEQ ID NO: 53) (aa 50 to aa 57 of SEQ ID NO: 12 or 13 or 14);
a heavy chain complementarity-determining region 2 (CDRH2) comprising, or consisting essentially of, or yet further consisting of a sequence of ISSGGSYT (SEQ ID NO: 54) (aa 75 to aa 82 of SEQ ID NO: 12 or 13 or 14);
a heavy chain complementarity-determining region 3 (CDRH3) comprising, or consisting essentially of, or yet further consisting of a sequence of ERHGGDGYWYFDV (SEQ ID NO: 55) (aa 121 to aa 133 of SEQ ID NO: 12 or 13 or 14);
a light chain complementarity-determining region 1 (CDRL1) comprising, or consisting essentially of, or yet further consisting of a sequence of QSLLDSDGKTF (SEQ ID NO: 51) (aa 47 to aa 57 of SEQ ID NO: 15 or 16 or 17 or 25);
a light chain complementarity-determining region 2 (CDRL2) comprising, or consisting essentially of, or yet further consisting of a sequence of LVS (aa 75 to aa 77 of SEQ ID NO: 15 or 16 or 17 or 25); and
a light chain complementarity-determining region 3 (CDRL3) comprising, or consisting essentially of, or yet further consisting of a sequence of WQGTHFPYT (SEQ ID NO: 56) (aa 114 to aa 122 of SEQ ID NO: 15 or 16 or 17 or 25).

In some embodiments, the antibody or fragment thereof that binds to a tip region of a DNABII peptide or a tip chimer comprises or consists essentially of, or yet further consists of or consists essentially of, or yet further consists of: a heavy chain (HC) immunoglobulin variable domain sequence comprising, or consisting essentially of, or yet further consisting of a sequence selected from the group of aa 25 to aa 144 of SEQ ID NO: 21 or 24, or an equivalent of each thereof, and/or a light chain (LC) immunoglobulin variable domain sequence comprising, or consisting essentially of, or yet further consisting of a sequence selected from the group of aa 21 to aa 132 of SEQ ID NO: 22 or 25 or an equivalent of each thereof. In certain embodiments, the antibody or fragment thereof binds to a DNABII peptide (such as the tip region of the DNABII peptide including but not limited to: a tip region of IHF or HU, a tip region of IHFA or IHFB, and/or the tip-chimeric peptide IhfA5-mIhfB4_{NTHI}). In one embodiment, the antibody or fragment thereof binds to a tip-chimeric peptide IhfA5-mIhfB4_{NTHI} as described herein. In some embodiments, a tip-chimeric peptide IhfA5-mIhfB4_{NTHI} comprises or consists essentially of, or yet further consists of an amino acid sequence selected from SEQ ID NOs: 26-28.

In some embodiments, the antibody or fragment thereof that binds to a tip region of a DNABII peptide comprises or consists essentially of, or yet further consists of: any one or any two or three heavy chain (HC) CDR comprising, or consisting essentially of, or yet further consisting of a sequence selected from SEQ ID NO: 9-14, or an equivalent of each thereof, and/or any one or any two or three light chain (LC) CDR comprising, or consisting essentially of, or yet further consisting of a sequence selected from 15-20 or an equivalent of each thereof. In certain embodiments, the antibody or fragment thereof binds to a DNABII peptide (such as the tip region of the DNABII peptide including but not limited to a tip region of IHF or HU, a tip region of IHFA or IHFB, and/or a tip-chimeric peptide IhfA5-mIhfB4_{NTHI} as described herein). In one embodiment, the antibody or fragment thereof binds to a tip-chimeric peptide IhfA5-mIhfB4_{NTHI} as described herein. In some embodiments, a tip-chimeric peptide IhfA5-mIhfB4_{NTHI} comprises or consists essentially of, or yet further consists of an amino acid sequence selected from SEQ ID NOs: 26-28.

In some embodiments, the antibody or fragment thereof that binds to a tip region of a DNABII peptide comprises or consists essentially of, or yet further consists of: all three heavy chain (HC) CDR comprising, or consisting essentially of, or yet further consisting of a sequence selected from SEQ ID NO: 9-11, or an equivalent of each thereof, and/or all three light chain (LC) CDR comprising, or consisting essentially of, or yet further consisting of a sequence selected from 15-17 or an equivalent of each thereof. In certain embodiments, the antibody or fragment thereof binds to a DNABII peptide (such as the tip region of the DNABII peptide including but not limited to a tip region of IHF or HU, a tip region of IHFA or IHFB, and/or a tip-chimeric peptide IhfA5-mIhfB4_{NTHI}). In one embodiment, the antibody or fragment thereof binds to a tip-chimeric peptide IhfA5-mIhfB4_{NTHI}. In some embodiments, a tip-chimeric peptide IhfA5-mIhfB4_{NTHI} comprises or consists essentially of, or yet further consists of an amino acid sequence selected from SEQ ID NOs: 26-28.

In some embodiments, the antibody or fragment thereof that binds to a tip region of a DNABII peptide comprises or consists essentially of, or yet further consists of: all three heavy chain (HC) CDR comprising, or consisting essentially of, or yet further consisting of a sequence selected from SEQ ID NO: 12-14, or an equivalent of each thereof, and/or all three light chain (LC) CDR comprising, or consisting essentially of, or yet further consisting of a sequence selected from 18-20 or an equivalent of each thereof. In certain embodiments, the antibody or fragment thereof binds to a DNABII peptide (such as the tip region of the DNABII peptide including but not limited to a tip region of IHF or HU, a tip region of IHFA or IHFB, and/or the tip-chimeric peptide IhfA5-mIhfB4_{NTHI}). In one embodiment, the antibody or fragment thereof binds to a tip-chimeric peptide IhfA5-mIhfB4_{NTHI}. In some embodiments, a tip-chimeric peptide IhfA5-mIhfB4_{NTHI} comprises or consists essentially of, or yet further consists of an amino acid sequence selected from SEQ ID NOs: 26-28.

Table 1 provides relative biofilm disruption data using humanized monoclonal antibodies designed to target IhfA5-mIhfB4_{NTHI} tip-chimeric peptide. Biofilm disruption: NTHI 86-028NP colonies were collected from overnight culture on chocolate agar and suspended in brain heart infusion broth supplemented with 2 μg β-NAD and heme per ml medium (sBHI). The optical density at 490 nm was then adjusted to 0.65 and the culture diluted 1:6 in sBHI prior to incubation at 37° C. with 5% CO2 for 3 hr, static. Next, the culture was diluted 1:2500 in fresh sBHI and 200 μl of the suspension aliquoted into each well of an 8-well chamber slide. The slide was then incubated at 37° C. with 5% CO₂ for 3 hr, static. After 16 hr, 200 μl fresh sBHI was added to each well, and the slide incubated an additional 8 hr. At this time point, medium was aspirated from each well and 5 μg monoclonal antibody added per well. The biofilms were incubated an additional 16 hr. Biofilms were then washed and stained with FM1-43FX bacterial cell membrane stain (Invitrogen) and fixed overnight at 4° C. in 16% paraformaldehyde, 2.5% glutaraldehyde, 4.0% acetic acid in 0.1 M phosphate buffer (pH 7.4). Fixative was aspirated and 200 μl Hank's Balanced Salt Solution was added to each well prior to viewing of biofilms on a Zeiss 800 Meta-laser scanning confocal microscope. Images were compiled with Zeiss Zen Black software and biofilm biomass calculated with COMSTAT2.1 software. The K_{A} for the IhfA5-mIhfB4_{NTHI} Tip chimeric peptide (in 1/M) is about 4E+05 to about 2E+08.

TABLE 1
Relative biofilm disruption data using different
humanized monoclonal antibodies designed to target
IhfA5-mIhfB4_{NTHI} tip-chimeric peptide

| | | | Biofilm |
|---|---|---|---|
| | | | disruption² |
| | | | (NCH) |
| | | | Value |
| | | | indicates |
| | | | remaining |
| | | | biomass |
| | | | (μm³/μm²) |
| | | | Nontypeable |
| Peptide | Heavy and light chain combination | | Haemophilus |
| target | Heavy chain | Light chain | influenzae |
| IhfA5- | HC1 (SEQ ID NO: 9) | LC1 (SEQ ID NO: 15) | 4.41 |
| mIhfB4 | HC1 (SEQ ID NO: 9) | LC2 (SEQ ID NO: 16) | 12.9 |
| Tip | HC1 (SEQ ID NO: 9) | LC3 (SEQ ID NO: 17) | 8.3 |
| chimeric | HC2 (SEQ ID NO: 10) | LC1 (SEQ ID NO: 15) | 7.1 |
| peptide | HC2 (SEQ ID NO: 10) | LC2 (SEQ ID NO: 16) | 6.9 |
| | HC2 (SEQ ID NO: 10) | LC3 (SEQ ID NO: 17) | 12.1 |
| | HC3 (SEQ ID NO: 11) | LC1 (SEQ ID NO: 15) | 7.4 |
| | HC3 (SEQ ID NO: 11) | LC2 (SEQ ID NO: 16) | 7.6 |
| | HC3 (SEQ ID NO: 11) | LC3 (SEQ ID NO: 17) | 14.1 |

In some embodiments, the antibody or fragment thereof that binds to a tip region of a DNABII peptide comprises or consists essentially of, or yet further consists of a heavy chain sequence comprising SEQ ID NO: 21 and a light chain sequence comprising SEQ ID NO: 22.

In some embodiments, the antibody or fragment thereof that binds to a tip region of a DNABII peptide comprises or consists essentially of, or yet further consists of a heavy chain sequence comprising SEQ ID NO: 9 and a light chain sequence comprising SEQ ID NO: 15.

In some embodiments, the antibody or fragment thereof that binds to a tip region of a DNABII peptide comprises or consists essentially of, or yet further consists of a heavy chain sequence comprising SEQ ID NO: 9 and a light chain sequence comprising SEQ ID NO: 16.

In some embodiments, the antibody or fragment thereof that binds to a tip region of a DNABII peptide comprises or consists essentially of, or yet further consists of a heavy chain sequence comprising SEQ ID NO: 9 and a light chain sequence comprising SEQ ID NO: 17.

In some embodiments, the antibody or fragment thereof that binds to a tip region of a DNABII peptide comprises or consists essentially of, or yet further consists of a heavy chain sequence comprising SEQ ID NO: 10 and a light chain sequence comprising SEQ ID NO: 15.

In some embodiments, the antibody or fragment thereof that binds to a tip region of a DNABII peptide comprises or consists essentially of, or yet further consists of a heavy chain sequence comprising SEQ ID NO: 10 and a light chain sequence comprising SEQ ID NO: 16.

In some embodiments, the antibody or fragment thereof that binds to a tip region of a DNABII peptide comprises or consists essentially of, or yet further consists of a heavy chain sequence comprising SEQ ID NO: 10 and a light chain sequence comprising SEQ ID NO: 17.

In some embodiments, the antibody or fragment thereof that binds to a tip region of a DNABII peptide comprises or consists essentially of, or yet further consists of a heavy chain sequence comprising SEQ ID NO: 11 and a light chain sequence comprising SEQ ID NO: 15.

In some embodiments, the antibody or fragment thereof that binds to a tip region of a DNABII peptide comprises or consists essentially of, or yet further consists of a heavy chain sequence comprising SEQ ID NO: 11 and a light chain sequence comprising SEQ ID NO: 16.

In some embodiments, the antibody or fragment thereof that binds to a tip region of a DNABII peptide comprises or consists essentially of, or yet further consists of a heavy chain sequence comprising SEQ ID NO: 11 and a light chain sequence comprising SEQ ID NO: 17.

In some embodiments, the antibody or fragment thereof that binds to a tip region of a DNABII peptide comprises or consists essentially of, or yet further consists of a heavy chain sequence comprising SEQ ID NO: 12 and a light chain sequence comprising SEQ ID NO: 18.

In some embodiments, the antibody or fragment thereof that binds to a tip region of a DNABII peptide comprises or consists essentially of, or yet further consists of a heavy chain sequence comprising SEQ ID NO: 12 and a light chain sequence comprising SEQ ID NO: 19.

In some embodiments, the antibody or fragment thereof that binds to a tip region of a DNABII peptide comprises or consists essentially of, or yet further consists of a heavy chain sequence comprising SEQ ID NO: 12 and a light chain sequence comprising SEQ ID NO: 20.

In some embodiments, the antibody or fragment thereof that binds to a tip region of a DNABII peptide comprises or consists essentially of, or yet further consists of a heavy chain sequence comprising SEQ ID NO: 13 and a light chain sequence comprising SEQ ID NO: 18.

In some embodiments, the antibody or fragment thereof that binds to a tip region of a DNABII peptide comprises or consists essentially of, or yet further consists of a heavy chain sequence comprising SEQ ID NO: 13 and a light chain sequence comprising SEQ ID NO: 19.

In some embodiments, the antibody or fragment thereof that binds to a tip region of a DNABII peptide comprises or consists essentially of, or yet further consists of a heavy chain sequence comprising SEQ ID NO: 13 and a light chain sequence comprising SEQ ID NO: 20.

In some embodiments, the antibody or fragment thereof that binds to a tip region of a DNABII peptide comprises or consists essentially of, or yet further consists of a heavy chain sequence comprising SEQ ID NO: 14 and a light chain sequence comprising SEQ ID NO: 18.

In some embodiments, the antibody or fragment thereof that binds to a tip region of a DNABII peptide comprises or consists essentially of, or yet further consists of a heavy chain sequence comprising SEQ ID NO: 14 and a light chain sequence comprising SEQ ID NO: 19.

In some embodiments, the antibody or fragment thereof that binds to a tip region of a DNABII peptide comprises or consists essentially of, or yet further consists of a heavy chain sequence comprising SEQ ID NO: 14 and a light chain sequence comprising SEQ ID NO: 20.

The antibody or fragment thereof as provided herein may be monospecific or bispecific. In one embodiment, the antibody or fragment thereof is trispecific, or tetraspecific, or pentaspecific. Additionally or alternatively, the antibody is selected from the group of an IgA (such as an IgA1 or an IgA2), an IgD, an IgE, an IgG (such as an IgG1, an IgG2, an IgG3, or an IgG4), or an IgM antibody. In one embodiment, the antibody further comprises a constant region selected from the group of: an IgA constant region (such as an IgA1 constant region or an IgA2 constant region), an IgD constant region, an IgE constant region, an IgG constant region (such as an IgG1 constant region, an IgG2 constant region, an IgG3 constant region, or an IgG4 constant region) or an IgM constant region. In some embodiments, the constant region of the antibody comprises an amino acid sequence selected from the group of SEQ ID NOs: 23, and 35-42.

In another aspect, the antibodies can be modified by conventional techniques, that may in one aspect increase the half-life of the antibody, e.g., PEGylation, a PEG mimetic, polysialylation, HESylation or glycosylation.

Further provided herein is a composition comprising, or consisting essentially of, or yet further consisting of an antibody or fragment thereof as described herein and an mB Box-97 peptide and optionally a carrier such as a pharmaceutically acceptable carrier. The mB Box peptide is a synthetic or recombinant polypeptide comprising, or consisting essentially of, or consisting of mB Box-97 that consists of the amino acids 90 to 176 or amino acids 80 to 176 from the coding sequence of the native human HMGB1 protein (as shown in SEQ ID NO: 2) with a cysteine to serine point mutation at amino acid 106 that abrogates the ability of this polypeptide to induce an inflammatory response as well as equivalents thereof that retain the cysteine to serine point mutation at amino acid 106 mB Box-97, consists of the amino acids 90 to 176 or amino acids 80 to 176 from the coding sequence of the native human HMGB1 protein (native/wild type human HMGB1 sequence is shown in SEQ ID NO: 2) that abrogate the ability of this polypeptide to induce an inflammatory response. The synthetic polypeptide is produced by synthetic or chemical means and is not wild type or produced recombinantly.

Methods to make the antibodies and fragments thereof are known in the art and described in U.S. Pat. No. 11,104,723, which is incorporated herein in its entirety.

### Compositions

Compositions are further provided. The compositions comprise a carrier and one or more of an isolated polypeptide disclosed herein, an isolated polynucleotide disclosed herein, a vector disclosed herein, an isolated host cell disclosed herein, a small molecule or an antibody, and/or an antigen binding fragment disclosed herein. The carriers can be one or more of a solid support or a pharmaceutically acceptable carrier. The compositions can further comprise an adjuvant or other components suitable for administrations as vaccines. In one aspect, the compositions are formulated with one or more pharmaceutically acceptable excipients, diluents, carriers and/or adjuvants. In addition, embodiments of the compositions of the present disclosure include one or more of an isolated polypeptide disclosed herein, an isolated polynucleotide disclosed herein, a vector disclosed herein, a small molecule, an isolated host cell disclosed herein, or an antibody of the disclosure, formulated with one or more pharmaceutically acceptable substances.

For oral preparations, any one or more of an isolated or recombinant polypeptide as described herein, an isolated or recombinant polynucleotide as described herein, a vector as described herein, an isolated host cell as described herein, a small molecule or an antibody or fragment thereof as described herein can be used alone or in pharmaceutical formulations disclosed herein comprising, or consisting essentially of, the compound in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The disclosure provides pharmaceutical formulations in which the one or more of an isolated polypeptide disclosed herein, an isolated polynucleotide disclosed herein, a vector disclosed herein, an isolated host cell disclosed herein, or an antibody disclosed herein can be formulated into preparations for injection in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents. A non-limiting example of such is a antimicrobial agent such as other vaccine components such as surface antigens, e.g., an OMP P5, OMP 26, OMP P2, or Type IV Pilin protein (see Jurcisek and Bakaletz (2007) J. of Bacteriology 189(10):3868-3875 and Murphy, T F, Bakaletz, L O and Smeesters, P R (2009) The Pediatric Infectious Disease Journal, 28:S121-S126) and antibacterial agents. For intravenous administration, suitable carriers include physiological bacteriostatic water, Cremophor EL™ (BASF, Parsippany, N.J.), or phosphate buffered saline (PBS). In all cases, a composition for parenteral administration must be sterile and should be fluid to the extent that easy syringability exists.

Aerosol formulations provided by the disclosure can be administered via inhalation and can be propellant or non-propellant based. For example, embodiments of the pharmaceutical formulations disclosed herein comprise a compound disclosed herein formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself, e.g., by an elastic bladder).

Suppositories disclosed herein can be prepared by mixing a compound disclosed herein with any of a variety of bases such as emulsifying bases or water-soluble bases. Embodiments of this pharmaceutical formulation of a compound disclosed herein can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds disclosed herein. Similarly, unit dosage forms for injection or intravenous administration may comprise a compound disclosed herein in a composition as a solution in sterile water, normal saline or another pharmaceutically acceptable carrier.

Embodiments of the pharmaceutical formulations disclosed herein include those in which one or more of an isolated polypeptide disclosed herein, an isolated polynucleotide disclosed herein, a vector disclosed herein, a small molecule for use in the disclosure, an isolated host cell disclosed herein, or an antibody or fragment thereof as disclosed herein is formulated in an injectable composition. Injectable pharmaceutical formulations disclosed herein are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations disclosed herein.

In an embodiment, one or more of an isolated polypeptide disclosed herein, an isolated polynucleotide disclosed herein, a vector disclosed herein, an isolated host cell disclosed herein, or an antibody disclosed herein is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound disclosed herein can be accomplished using any of a variety of refillable, pump systems. Pumps provide consistent, controlled release over time. In some embodiments, a compound disclosed herein is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation, polymers such as for example poly(glycolide-co-lactide) (PGLA) that is commercially available from a number of vendors, e.g., BioDegmer and Sigma-Aldrich. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer (e.g., PGLA) and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852; and the like. In general, a subject treatment method can be accomplished using any of a variety of refillable, non-exchangeable pump systems. Pumps and other convective systems may be utilized due to their generally more consistent, controlled release over time. Osmotic pumps are used in some embodiments due to their combined advantages of more consistent controlled release and relatively small size (see, e.g., PCT International Application Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a compound disclosed herein are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carbocylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the polypeptide, antibody or fragment thereof (as well as combination compositions) is delivered in a controlled release system. For example, a compound disclosed herein may be administered using intravenous infusion, an implantable osmotic pump, a transdermal patch, liposomes, or other modes of administration. In one embodiment, a pump may be used (Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the liver, thus requiring only a fraction of the systemic dose. In yet another embodiment, a controlled release system is placed in proximity of the therapeutic target, thus requiring only a fraction of the systemic. Other controlled release systems are discussed in the review by Langer (1990) Science 249:1527-1533.

In another embodiment, the compositions of the present disclosure (as well as combination compositions separately or together) include those formed by impregnation of an inhibiting agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

The present disclosure provides methods and compositions for the administration of a one or more of an interfering agent to a host (e.g., a human) for the treatment of a microbial infection. In various embodiments, these methods disclosed herein span almost any available method and route suitable for drug delivery, including in vivo and ex vivo methods, as well as systemic and localized routes of administration.

### Methods Using an mB Box-97 Polypeptide

Another aspect of the disclosure is directed to a method for treating a subject in need thereof comprising, or consisting essentially of, or yet further consisting of administering to the subject an effective amount of one or more of: (i) the synthetic or recombinant polypeptide or the equivalent of each thereof of the present disclosure, (ii) the plurality of the present disclosure, (iii) the composition of the present disclosure, (iv) the isolated polynucleotide of the present disclosure, or (iv) the vector of the present disclosure.

Another aspect of the disclosure is directed to a method for treating or preventing aberrant or excessive Neutrophil Extracellular Trap (NET) formation or to prevent a Neutrophil Extracellular Trap (NET)-mediated disease or prevent the progression of NET-mediated disease in a subject in need thereof comprising, or consisting essentially of, or yet further consisting of administering to the subject an effective amount of one or more of: (i) the synthetic or recombinant polypeptide or the equivalent of each thereof of the present disclosure, (ii) the plurality of the present disclosure, (iii) the composition of the present disclosure, (iv) the isolated polynucleotide of the present disclosure, or (iv) the vector of the present disclosure.

In some embodiments, the subject is suffering for one or more of a pulmonary disease selected from SARS CoV-2, cystic fibrosis, asthma, chronic obstructive pulmonary disease, tuberculosis, bacterial pneumonia, respiratory syncytial virus bronchiolitis, influenza virus infection, transfusion-related acute lung injury, and mechanical ventilation; sepsis; atherosclerosis; an autoimmune disease selected from systemic lupus erythematosus, rheumatoid arthritis, Type I diabetes mellitus, or small vessel vasculitis; an autoinflammatory disease selected from gout or inflammatory bowel disease, and/or a metabolic disease selected from Type 2 diabetes or obesity.

In some embodiments, the effective amount is between 50 nM and 2 μM (e.g., 50 nM, 60 nM, 70 nM, 80 nM, 90 nM, 100 nM, 120 nM, 150 nM, 170 nM, 200 nM, 220 nM, 250 nM, 270 nM, 300 nM, 350 nM, 400 nM, 450 nM, 500 nM, 600 nM, 650 nM, 700 nM, 750 nM, 800 nM, 850 nM, 900 nM, 950 nM, 1000 nM (1 μM), 1050 nM, 1100 nM, 1150 nM, 1200 nM, 1250 nM, 1300 nM, 1350 nM, 1400 nM, 1450 nM, 1500 nM, 1550 nM, 1600 nM, 1650 nM, 1700 nM, 1750 nM, 1800 nM, 1850 nM, 1900 nM, 1950 nM, or 2000 nM (2 μM)).

In some embodiments, the NET-mediated disease comprises or consists essentially of, or yet further consists of a pulmonary disease selected from SARS CoV-2, cystic fibrosis, asthma, chronic obstructive pulmonary disease, tuberculosis, bacterial pneumonia, respiratory syncytial virus bronchiolitis, influenza virus infection, transfusion-related acute lung injury, and mechanical ventilation; sepsis; atherosclerosis; an autoimmune disease selected from systemic lupus erythematosus, rheumatoid arthritis, Type I diabetes mellitus, or small vessel vasculitis; an autoinflammatory disease selected from gout or inflammatory bowel disease, or a metabolic disease selected from Type 2 diabetes or obesity.

Another aspect of the disclosure is directed to a method for preventing or treating a bacterial biofilm in a subject in need thereof comprising, or consisting essentially of, or yet further consisting of administering to the subject an effective amount of one or more of: (i) the synthetic or recombinant polypeptide or the equivalent of each thereof of the present disclosure, (ii) the plurality of the present disclosure, (iii) the composition of the present disclosure, (iv) the isolated polynucleotide of the present disclosure, or (iv) the vector of the present disclosure.

In some embodiments, the effective amount is between 50 nM and 2 μM (e.g., 50 nM, 60 nM, 70 nM, 80 nM, 90 nM, 100 nM, 120 nM, 150 nM, 170 nM, 200 nM, 220 nM, 250 nM, 270 nM, 300 nM, 350 nM, 400 nM, 450 nM, 500 nM, 600 nM, 650 nM, 700 nM, 750 nM, 800 nM, 850 nM, 900 nM, 950 nM, 1000 nM (1 μM), 1050 nM, 1100 nM, 1150 nM, 1200 nM, 1250 nM, 1300 nM, 1350 nM, 1400 nM, 1450 nM, 1500 nM, 1550 nM, 1600 nM, 1650 nM, 1700 nM, 1750 nM, 1800 nM, 1850 nM, 1900 nM, 1950 nM, or 2000 nM (2 μM)).

In some embodiments, the method further comprises or consists essentially of, or yet further consists of administering to the subject an antibody or a fragment thereof, wherein the antibody or fragment thereof binds to a tip region of a DNABII peptide, for example (IhfA5-mIhfB4_{NTHI} tip chimer). In one aspect the antibody is a monoclonal antibody, a humanized antibody, or an antigen-binding fragment thereof

In some embodiments, the antibody or a fragment thereof that binds to a tip region of a DNABII peptide or a tip chimer, the antibody or fragment thereof comprises:
(iii) a heavy chain (HC) immunoglobulin variable domain sequence comprising, or consisting essentially of, or yet further consisting of a sequence of amino acid (aa) 25 to aa 144 of SEQ ID NO: 21 or an equivalent thereof; and
(iv) a light chain (LC) immunoglobulin variable domain sequence comprising, or consisting essentially of, or yet further consisting of a sequence of aa 21 to aa 132 of SEQ ID NO: 22 or an equivalent thereof;
   or
   wherein the antibody or a fragment thereof comprises:
(iii) a heavy chain (HC) immunoglobulin variable domain sequence comprising, or consisting essentially of, or yet further consisting of a sequence of aa 25 to aa 144 of SEQ ID NO: 24 or an equivalent thereof; and
(iv) a light chain (LC) immunoglobulin variable domain sequence comprising, or consisting essentially of, or yet further consisting of a sequence of aa 21 to aa 132 of SEQ ID NO: 25 or an equivalent thereof.

In some embodiments, the antibody or a fragment thereof that binds to a tip region of a DNABII peptide comprises:
a heavy chain complementarity-determining region 1 (CDRH1) comprising, or consisting essentially of, or yet further consisting of a sequence of GFTFRTY (SEQ ID NO: 47) (aa 50 to aa 56 of SEQ ID NO: 9 or 10 or 11 or 24);
a heavy chain complementarity-determining region 2 (CDRH2) comprising, or consisting essentially of, or yet further consisting of a sequence of GSDRRH (SEQ ID NO: 48) (aa 76 to aa 81 of SEQ ID NO: 9 or 10 or 11 or 24);
a heavy chain complementarity-determining region 3 (CDRH3) comprising, or consisting essentially of, or yet further consisting of a sequence of VGPYDGYYGEFDY (SEQ ID NO: 49) (aa 121 to aa 133 of SEQ ID NO: 9 or 10 or 11 or 24);
a light chain complementarity-determining region 1 (CDRL1) comprising, or consisting essentially of, or yet further consisting of a sequence of QSLLDSDGKTF (SEQ ID NO: 51) (aa 47 to aa 57 of SEQ ID NO: 15 or 16 or 17 or 25);
a light chain complementarity-determining region 2 (CDRL2) comprising, or consisting essentially of, or yet further consisting of a sequence of LVS (aa 75 to aa 77 of SEQ ID NO: 15 or 16 or 17 or 25); and
a light chain complementarity-determining region 3 (CDRL3) comprising, or consisting essentially of, or yet further consisting of a sequence of WQGTHFP (SEQ ID NO: 52) (aa 114 to aa 120 of SEQ ID NO: 15 or 16 or 17 or 25).

In some embodiments, the antibody or fragment thereof that binds to a tip region of a DNABII peptide comprises or consists essentially of, or yet further consists of or consists essentially of, or yet further consists of: a heavy chain (HC) immunoglobulin variable domain sequence comprising, or consisting essentially of, or yet further consisting of a sequence selected from the group of aa 25 to aa 144 of SEQ ID NO: 21 or 24, or an equivalent of each thereof, and/or a light chain (LC) immunoglobulin variable domain sequence comprising, or consisting essentially of, or yet further consisting of a sequence selected from the group of aa 21 to aa 132 of SEQ ID NO: 22 or 25 or an equivalent of each thereof. In certain embodiments, the antibody or fragment thereof binds to a DNABII peptide (such as the tip region of the DNABII peptide including but not limited to: a tip region of IHF or HU, a tip region of IHFA or IHFB, and/or the tip-chimeric peptide IhfA5-mIhfB4_{NTHI}). In one embodiment, the antibody or fragment thereof binds to the tip-chimeric peptide IhfA5-mIhfB4_{NTHI}. As used herein, tip-chimeric peptide IhfA5-mIhfB4_{NTHI} comprises or consists essentially of, or yet further consists of an amino acid sequence selected from SEQ ID NOs: 26-28.

In some embodiments, the antibody or fragment thereof that binds to a tip region of a DNABII peptide comprises or consists essentially of, or yet further consists of: any one or any two or three heavy chain (HC) CDR comprising, or consisting essentially of, or yet further consisting of a sequence selected from SEQ ID NO: 9-14, or an equivalent of each thereof, and/or any one or any two or three light chain (LC) CDR comprising, or consisting essentially of, or yet further consisting of a sequence selected from 15-20 or an equivalent of each thereof. In certain embodiments, the antibody or fragment thereof binds to a DNABII peptide (such as the tip region of the DNABII peptide including but not limited to: a tip region of IHF or HU, a tip region of IHFA or IHFB, and/or the tip-chimeric peptide IhfA5-mIhfB4_{NTHI}). In one embodiment, the antibody or fragment thereof binds to the tip-chimeric peptide IhfA5-mIhfB4_{NTHI}.

In some embodiments, the antibody or fragment thereof that binds to a tip region of a DNABII peptide comprises or consists essentially of, or yet further consists of: all three heavy chain (HC) CDR comprising, or consisting essentially of, or yet further consisting of a sequence selected from SEQ ID NO: 9-11, or an equivalent of each thereof, and/or all three light chain (LC) CDR comprising, or consisting essentially of, or yet further consisting of a sequence selected from 15-17 or an equivalent of each thereof. In certain embodiments, the antibody or fragment thereof binds to a DNABII peptide (such as the tip region of the DNABII peptide including but not limited to: a tip region of IHF or HU, a tip region of IHFA or IHFB, and/or the tip-chimeric peptide IhfA5-mIhfB4_{NTHI}). In one embodiment, the antibody or fragment thereof binds to the tip-chimeric peptide IhfA5-mIhfB4_{NTHI}.

In some embodiments, the antibody or fragment thereof that binds to a tip region of a DNABII peptide comprises or consists essentially of, or yet further consists of: all three heavy chain (HC) CDR comprising, or consisting essentially of, or yet further consisting of a sequence selected from SEQ ID NO: 12-14, or an equivalent of each thereof, and/or all three light chain (LC) CDR comprising, or consisting essentially of, or yet further consisting of a sequence selected from 18-20 or an equivalent of each thereof. In certain embodiments, the antibody or fragment thereof binds to a DNABII peptide (such as the tip region of the DNABII peptide including but not limited to: a tip region of IHF or HU, a tip region of IHFA or IHFB, and/or the tip-chimeric peptide IhfA5-mIhfB4_{NTHI}). In one embodiment, the antibody or fragment thereof binds to the tip-chimeric peptide IhfA5-mIhfB4_{NTHI}.

In some embodiments, the antibody or fragment thereof that binds to a tip region of a DNABII peptide comprises or consists essentially of, or yet further consists of a heavy chain sequence comprising SEQ ID NO: 21 and a light chain sequence comprising SEQ ID NO: 22.

In some embodiments, the antibody or fragment thereof that binds to a tip region of a DNABII peptide comprises or consists essentially of, or yet further consists of a heavy chain sequence comprising SEQ ID NO: 9 and a light chain sequence comprising SEQ ID NO: 15.

In some embodiments, the antibody or fragment thereof that binds to a tip region of a DNABII peptide comprises or consists essentially of, or yet further consists of a heavy chain sequence comprising SEQ ID NO: 9 and a light chain sequence comprising SEQ ID NO: 16.

In some embodiments, the antibody or fragment thereof that binds to a tip region of a DNABII peptide comprises or consists essentially of, or yet further consists of a heavy chain sequence comprising SEQ ID NO: 9 and a light chain sequence comprising SEQ ID NO: 17.

In some embodiments, the antibody or fragment thereof that binds to a tip region of a DNABII peptide comprises or consists essentially of, or yet further consists of a heavy chain sequence comprising SEQ ID NO: 10 and a light chain sequence comprising SEQ ID NO: 15.

In some embodiments, the antibody or fragment thereof that binds to a tip region of a DNABII peptide comprises or consists essentially of, or yet further consists of a heavy chain sequence comprising SEQ ID NO: 10 and a light chain sequence comprising SEQ ID NO: 16.

In some embodiments, the antibody or fragment thereof that binds to a tip region of a DNABII peptide comprises or consists essentially of, or yet further consists of a heavy chain sequence comprising SEQ ID NO: 10 and a light chain sequence comprising SEQ ID NO: 17.

In some embodiments, the antibody or fragment thereof that binds to a tip region of a DNABII peptide comprises or consists essentially of, or yet further consists of a heavy chain sequence comprising SEQ ID NO: 11 and a light chain sequence comprising SEQ ID NO: 15.

In some embodiments, the antibody or fragment thereof that binds to a tip region of a DNABII peptide comprises or consists essentially of, or yet further consists of a heavy chain sequence comprising SEQ ID NO: 11 and a light chain sequence comprising SEQ ID NO: 16.

In some embodiments, the antibody or fragment thereof that binds to a tip region of a DNABII peptide comprises or consists essentially of, or yet further consists of a heavy chain sequence comprising SEQ ID NO: 11 and a light chain sequence comprising SEQ ID NO: 17.

In some embodiments, the antibody or fragment thereof that binds to a tip region of a DNABII peptide comprises or consists essentially of, or yet further consists of a heavy chain sequence comprising SEQ ID NO: 12 and a light chain sequence comprising SEQ ID NO: 18.

In some embodiments, the antibody or fragment thereof that binds to a tip region of a DNABII peptide comprises or consists essentially of, or yet further consists of a heavy chain sequence comprising SEQ ID NO: 12 and a light chain sequence comprising SEQ ID NO: 19.

In some embodiments, the antibody or fragment thereof that binds to a tip region of a DNABII peptide comprises or consists essentially of, or yet further consists of a heavy chain sequence comprising SEQ ID NO: 12 and a light chain sequence comprising SEQ ID NO: 20.

In some embodiments, the antibody or fragment thereof that binds to a tip region of a DNABII peptide comprises or consists essentially of, or yet further consists of a heavy chain sequence comprising SEQ ID NO: 13 and a light chain sequence comprising SEQ ID NO: 18.

In some embodiments, the antibody or fragment thereof that binds to a tip region of a DNABII peptide comprises or consists essentially of, or yet further consists of a heavy chain sequence comprising SEQ ID NO: 13 and a light chain sequence comprising SEQ ID NO: 19.

In some embodiments, the antibody or fragment thereof that binds to a tip region of a DNABII peptide comprises or consists essentially of, or yet further consists of a heavy chain sequence comprising SEQ ID NO: 13 and a light chain sequence comprising SEQ ID NO: 20.

In some embodiments, the antibody or fragment thereof that binds to a tip region of a DNABII peptide comprises or consists essentially of, or yet further consists of a heavy chain sequence comprising SEQ ID NO: 14 and a light chain sequence comprising SEQ ID NO: 18.

In some embodiments, the antibody or fragment thereof that binds to a tip region of a DNABII peptide comprises or consists essentially of, or yet further consists of a heavy chain sequence comprising SEQ ID NO: 14 and a light chain sequence comprising SEQ ID NO: 19.

In some embodiments, the antibody or fragment thereof that binds to a tip region of a DNABII peptide comprises or consists essentially of, or yet further consists of a heavy chain sequence comprising SEQ ID NO: 14 and a light chain sequence comprising SEQ ID NO: 20.

The antibody or fragment thereof as provided herein may be monospecific or bispecific. In one embodiment, the antibody or fragment thereof is trispecific, or tetraspecific, or pentaspecific. Additionally or alternatively, the antibody is selected from the group of an IgA (such as an IgA1 or an IgA2), an IgD, an IgE, an IgG (such as an IgG1, an IgG2, an IgG3, or an IgG4), or an IgM antibody. In one embodiment, the antibody further comprises a constant region selected from the group of: an IgA constant region (such as an IgA1 constant region or an IgA2 constant region), an IgD constant region, an IgE constant region, an IgG constant region (such as an IgG1 constant region, an IgG2 constant region, an IgG3 constant region, or an IgG4 constant region) or an IgM constant region. In some embodiments, the constant region of the antibody comprises an amino acid sequence selected from the group of SEQ ID NOs: 23, and 35-42.

In another aspect, the antibodies can be modified by conventional techniques, that may in one aspect increase the half-life of the antibody, e.g., PEGylation, a PEG mimetic, polysialylation, HESylation or glycosylation.

Methods for Condensing eDNA tendrils of a Neutrophil Extracellular Traps (NET)

Another aspect of the disclosure is directed to a method for condensing eDNA tendrils of a Neutrophil Extracellular Traps (NET) comprising, or consisting essentially of, or yet further consisting of contacting the NET with an effective amount of a DNA-binding agent.

Another aspect of the disclosure is directed to a method for preventing or disabling the eDNA structure of a Neutrophil Extracellular Traps (NET) comprising, or consisting essentially of, or yet further consisting of contacting the NET with an effective amount of a DNA-binding agent.

Another aspect of the disclosure is directed to a method for preventing a Neutrophil Extracellular Trap (NET) formation or inducing retraction of extant NETs comprising, or consisting essentially of, or yet further consisting of contacting the NET with an effective amount of a DNA-binding agent.

In some embodiments, the DNA-binding agent is an agent that aggregates or condenses DNA.

In some embodiments, the effective amount is between 50 nM and 2 μM (e.g., 50 nM, 60 nM, 70 nM, 80 nM, 90 nM, 100 nM, 120 nM, 150 nM, 170 nM, 200 nM, 220 nM, 250 nM, 270 nM, 300 nM, 350 nM, 400 nM, 450 nM, 500 nM, 600 nM, 650 nM, 700 nM, 750 nM, 800 nM, 850 nM, 900 nM, 950 nM, 1000 nM (1 μM), 1050 nM, 1100 nM, 1150 nM, 1200 nM, 1250 nM, 1300 nM, 1350 nM, 1400 nM, 1450 nM, 1500 nM, 1550 nM, 1600 nM, 1650 nM, 1700 nM, 1750 nM, 1800 nM, 1850 nM, 1900 nM, 1950 nM, or 2000 nM (2 μM)).

In some embodiments, the DNA-binding agent comprises or consists essentially of, or yet further consists of a Histone-like Nucleoid Structuring protein (H-NS), a polyamine, or a polycation.

In some embodiments, the H-NS is derived from a gram-negative bacterium or a gram-positive bacterium. In some embodiments, the H-NS is derived from a bacterium of a genus Escherichia, Haemophilus, Streptococcus, Mycobacteria, Klebsiella, or Pseudomonas; optionally wherein the H-NS is derived from E Coli, Nontippable Haemophilus influenzae (NTHI), S. pneumoniae, K. pneumoniae, Mycobacterium tuberculosis, or Pseudomonas aeruginosa.

In some embodiments, the H-NS comprises or consists essentially of, or yet further consists of an amino acid sequence having at least 60% (e.g., at least 60, 65, 70, 75, 80, 85, 90, 95, 99% or more) identity to an amino acid sequence selected from SEQ ID NOs: 29-34, the equivalent is identical to the reference polypeptide. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

In some embodiments, the contacting is in vitro or in vivo.

Another aspect of the disclosure is directed to a method for arresting damaging clotting in a subject in need comprising, or consisting essentially of, or yet further consisting of administering to the subject in need an effective amount of a DNA-binding agent.

In some embodiments, the DNA-binding agent comprises or consists essentially of, or yet further consists of a Histone-like Nucleoid Structuring protein (H-NS), a polyamine, or a polycation.

In some embodiments, the H-NS is derived from a gram-negative bacterium or a gram-positive bacterium. In some embodiments, the H-NS is derived from a bacterium of a genus Escherichia, Haemophilus, Streptococcus, Mycobacteria, Klebsiella, or Pseudomonas; optionally wherein the H-NS is derived from E Coli, Nontypeable Haemophilus influenzae (NTHI), S. pneumoniae, K. pneumoniae, Mycobacterium tuberculosis, or Pseudomonas aeruginosa.

In some embodiments, the H-NS comprises or consists essentially of, or yet further consists of an amino acid sequence having at least 60% (e.g., at least 60, 65, 70, 75, 80, 85, 90, 95, 99% or more) identity to an amino acid sequence selected from SEQ ID NOs: 29-34, the equivalent is identical to the reference polypeptide. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

Another aspect of the disclosure is directed to a method for arresting excessive inflammation in a subject in need comprising, or consisting essentially of, or yet further consisting of administering to the subject in need an effective amount of a DNA-binding agent, optionally wherein the effective amount is between 50 nM and 2 μM.

Another aspect of the disclosure is directed to a method for preventing, treating or preventing the progression of a Neutrophil Extracellular Trap (NET)-mediated disease in a subject in need comprising, or consisting essentially of, or yet further consisting of administering to the subject in need an effective amount of a DNA-binding agent. In some embodiments, the effective amount is between 50 nM and 2 μM (e.g., 50 nM, 60 nM, 70 nM, 80 nM, 90 nM, 100 nM, 120 nM, 150 nM, 170 nM, 200 nM, 220 nM, 250 nM, 270 nM, 300 nM, 350 nM, 400 nM, 450 nM, 500 nM, 600 nM, 650 nM, 700 nM, 750 nM, 800 nM, 850 nM, 900 nM, 950 nM, 1000 nM (1 μM), 1050 nM, 1100 nM, 1150 nM, 1200 nM, 1250 nM, 1300 nM, 1350 nM, 1400 nM, 1450 nM, 1500 nM, 1550 nM, 1600 nM, 1650 nM, 1700 nM, 1750 nM, 1800 nM, 1850 nM, 1900 nM, 1950 nM, or 2000 nM (2 μM)).

In some embodiments, the DNA-binding agent is an agent that aggregates or condenses DNA. In some embodiments, the effective amount is between 50 nM and 2 μM (e.g., 50 nM, 60 nM, 70 nM, 80 nM, 90 nM, 100 nM, 120 nM, 150 nM, 170 nM, 200 nM, 220 nM, 250 nM, 270 nM, 300 nM, 350 nM, 400 nM, 450 nM, 500 nM, 600 nM, 650 nM, 700 nM, 750 nM, 800 nM, 850 nM, 900 nM, 950 nM, 1000 nM (1 μM), 1050 nM, 1100 nM, 1150 nM, 1200 nM, 1250 nM, 1300 nM, 1350 nM, 1400 nM, 1450 nM, 1500 nM, 1550 nM, 1600 nM, 1650 nM, 1700 nM, 1750 nM, 1800 nM, 1850 nM, 1900 nM, 1950 nM, or 2000 nM (2 μM)).

In some embodiments, the DNA-binding agent comprises or consists essentially of, or yet further consists of a Histone-like Nucleoid Structuring protein (H-NS), a polyamine, or a polycation.

In some embodiments, the H-NS is derived from a gram-negative bacterium or a gram-positive bacterium. In some embodiments, the H-NS is derived from a bacterium of a genus Escherichia, Haemophilus, Streptococcus, Mycobacteria, Klebsiella, or Pseudomonas; optionally wherein the H-NS is derived from E Coli, Nontypeable Haemophilus influenzae (NTHI), S. pneumoniae, K. pneumoniae, Mycobacterium tuberculosis, or Pseudomonas aeruginosa.

In some embodiments, the H-NS comprises or consists essentially of, or yet further consists of an amino acid sequence having at least 60% (e.g., at least 60, 65, 70, 75, 80, 85, 90, 95, 99% or more) identity to an amino acid sequence selected from SEQ ID NOs: 29-34.

In some embodiments, the H-NS comprises or consists essentially of, or yet further consists of an amino acid sequence having at least 60% (e.g., at least 60, 65, 70, 75, 80, 85, 90, 95, 99% or more) identity to an amino acid sequence selected from SEQ ID NOs: 29-34, the equivalent is identical to the reference polypeptide. In one aspect, the percent identity is determined using the BLAST alignment program, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

In some embodiments, the subject is a mammal or a human patient.

In some embodiments, the subject is suffering for one or more of a pulmonary disease selected from SARS CoV-2, cystic fibrosis, asthma, chronic obstructive pulmonary disease, tuberculosis, bacterial pneumonia, respiratory syncytial virus bronchiolitis, influenza virus infection, transfusion-related acute lung injury, and mechanical ventilation; sepsis; atherosclerosis; an autoimmune disease selected from systemic lupus erythematosus, rheumatoid arthritis, Type I diabetes mellitus, or small vessel vasculitis; an autoinflammatory disease selected from gout or inflammatory bowel disease, and/or a metabolic disease selected from Type 2 diabetes or obesity.

### Combination Therapy

The compositions and related methods of the present disclosure may be used in combination with the administration of other therapies. These include, but are not limited to, the administration of DNase enzymes, antibiotics, antimicrobials, anti-infectives, anti-fungals, anti-parasitics, anti-virals, or other antibodies.

In some embodiments, the methods and compositions include a deoxyribonuclease (DNase) enzyme that acts synergistically with the anti-DNABII antibody. A DNase is any enzyme that catalyzes the cleavage of phosphodiester linkages in the DNA backbone. Three non-limiting examples of DNase enzymes that are known to target not only cruciform structures, but also a variety of secondary structure of DNA include DNAse I, T4 EndoVII, T7 Endo I, RuvABC, and RusA. In certain embodiments, the effective amount of anti-DNABII antibody needed to destabilize the biofilm is reduced when combined with a DNase. When administered in vitro, the DNase can be added directly to the assay or in a suitable buffer known to stabilize the enzyme. The effective Unit dose of DNase and the assay conditions may vary, and can be optimized according to procedures known in the art.

In other embodiments, the methods and compositions can be combined with antibiotics and/or antimicrobials. Antimicrobials are substances that kill or inhibit the growth of microorganisms such as bacteria, fungi, or protozoans. Although biofilms are generally resistant to the actions of antibiotics, compositions and methods described herein can be used to sensitize the infection involving a biofilm to traditional therapeutic methods for treating infections. In other embodiments, the use of antibiotics or antimicrobials in combination with methods and compositions described herein allow for the reduction of the effective amount of the antimicrobial and/or biofilm reducing agent. Some non-limiting examples of antimicrobials and antibiotics useful in combination with methods of the current disclosure include amoxicillin, amoxicillin-clavulanate, cefdinir, azithromycin, and sulfamethoxazole-trimethoprim. The therapeutically effective dose of the antimicrobial and/or antibiotic in combination with the biofilm reducing agent can be readily determined by traditional methods. In some embodiments the dose of the antimicrobial agent in combination with the biofilm reducing agent is the average effective dose which has been shown to be effective in other bacterial infections, for example, bacterial infections wherein the etiology of the infection does not include a biofilm. In other embodiments, the dose is 0.1, 0.15, 0.2, 0.25, 0,30, 0,35, 0.40, 0.45, 0.50, 0.55, 0.60, 0.65, 0.70, 0.75, 0.8, 0.85, 0.9, 0.95, 1.1, 1.2, 1.3, 1.4, 1.5, 1.6, 1.7, 1.8, 1.9, 2.0, 2.5, 3.0 or 5 times the average effective dose. The antibiotic or antimicrobial can be added prior to, concurrent with, or subsequent to the addition of the anti-DNABII antibody.

In other embodiments, the methods and compositions can be combined with antibodies that treat the bacterial infection. One example of an antibody useful in combination with the methods and compositions described herein is an antibody directed against an unrelated outer membrane protein (i.e., OMP P5). Treatment with this antibody alone does not debulk a biofilm in vitro. Combined therapy with this antibody and a biofilm reducing agent results in a greater effect than that which could be achieved by either reagent used alone at the same concentration. Other antibodies that may produce a synergistic effect when combined with a biofilm reducing agent or methods to reduce a biofilm include anti-rsPilA anti-OMP26, anti-OMP P2, and anti-whole OMP preparations.

The compositions and methods described herein can be used to sensitize the bacterial infection involving a biofilm to common therapeutic modalities effective in treating bacterial infections without a biofilm but are otherwise ineffective in treating bacterial infections involving a biofilm. In other embodiments, the compositions and methods described herein can be used in combination with therapeutic modalities that are effective in treating bacterial infections involving a biofilm, but the combination of such additional therapy and biofilm reducing agent or method produces a synergistic effect such that the effective dose of either the biofilm reducing agent or the additional therapeutic agent can be reduced. In other instances, the combination of such additional therapy and biofilm reducing agent or method produces a synergistic effect such that the treatment is enhanced. An enhancement of treatment can be evidenced by a shorter amount of time required to treat the infection.

The additional therapeutic treatment can be added prior to, concurrent with, or subsequent to methods or compositions used to reduce the biofilm, and can be contained within the same formation/composition or as a separate formulation/composition.

### Kits

Kits containing one or more of the polypeptides, antibodies or fragments thereof or the agents and instructions necessary to perform the in vitro and in vivo methods as described herein also are disclosed. Accordingly, the disclosure provides kits for performing these methods which may include an antibody, antibody fragment, polypeptide, polynucleotide, vector or host cell, as well as instructions for carrying out the methods disclosed herein such as collecting tissue and/or performing the screen, and/or analyzing the results, and/or administration of an effective amount of an antibody, antibody fragment, polypeptide, polynucleotide, vector or host cell, as defined herein. These can be used alone or in combination with other suitable antimicrobial agents.

For example, a kit can comprise, or alternatively consist essentially of, or yet further consist of any one or more of agents identified above, e.g., antibody, antibody fragment, polypeptide, polynucleotide, vector or host cell, and instructions for use. The kit can further comprise one or more of an adjuvant, an antigenic peptide or an antimicrobial. Examples of carriers include a liquid carrier, a pharmaceutically acceptable carrier, a solid phase carrier, a pharmaceutically acceptable carrier, a pharmaceutically acceptable polymer, a liposome, a micelle, an implant, a stent, a paste, a gel, a dental implant, or a medical implant.

### EXAMPLES

The following examples are intended to illustrate, but not limit the scope of the disclosure.

### Example 1: MB Box-97 Peptide and Inhibition of NETosis

### Only mB Box-97, Amongst all HMGB1-Derived Constructs, Shows Inhibition of NETosis in Isolated Human Neutrophils.

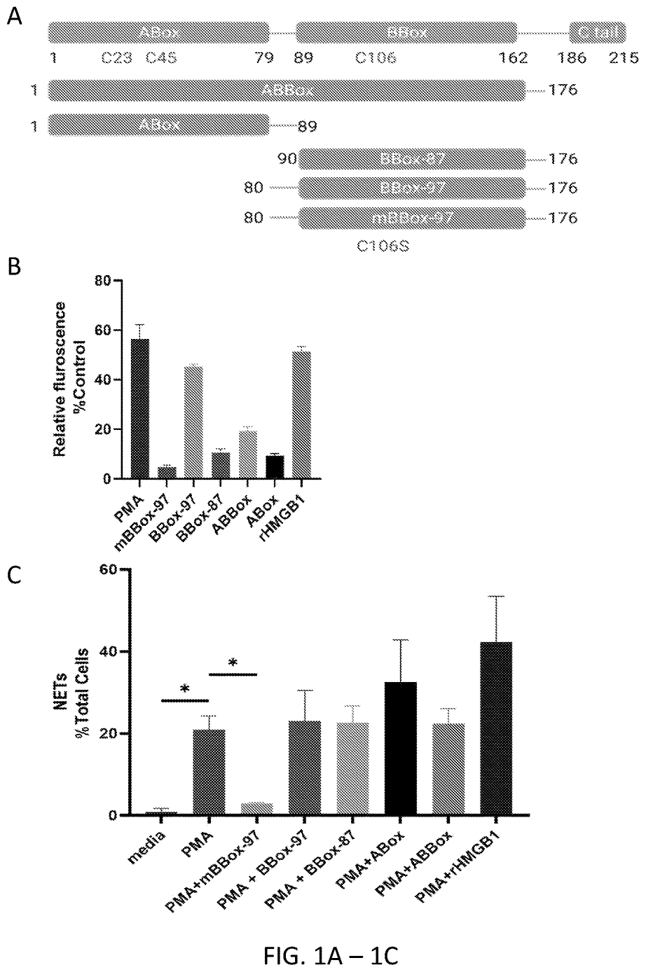

HMGB1 is a known inducer of inflammation and NETosis. However, previously Applicant showed that an engineered point mutation (C45S; mHMGB-1) strongly reduced the proinflammatory functions of HMGB1. Indeed, mHMGB1 was able to reduce the neutrophil migration to the peritoneal cavity upon induction of peritoneal inflammation by Thioglacolate suggesting mHMGB1 induces attenuated inflammatory neutrophil response. mHMGB1 was also able to reduce the number of neutrophils in bronchoalveolar lavage (BAL) in mice infected with B. cenocepacia. In other work, a synthetic peptide synthesized comprising just BoxA of HMGB1 was shown to affect macrophage function and could attenuate liver damage in a cirrhosis mouse model. These observations suggest that HMGB1 has the potential to affect neutrophils and that some of these functions reside in specific domains. Indeed, Applicant had made a series of truncation constructs of HMGB1 and found that an extended B-Box domain (B Box 97) alone possessed the anti-biofilm activity equivalent to the whole protein. Moreover, Applicant showed that a single point mutation in the B Box (C106S) that is known to reduce the proinflammatory activity of HMGB1 had identical antibiofilm activity. Here, Applicant used various truncated constructs of HMGB1 to determine if any portion of the protein specifically affected neutrophil functions. Recombinant full-length HMGB1, ABox (amino acids 1 to 89), ABBox (amino acids 1 to 176), BBox-97 (amino acids 80 to 176), recombinant and synthetic mB Box-97 (identical to BBox-97 except for a single amino acid change C106S) and BBox-87 (amino acids 90 to 176) (FIG. 1A) were each tested peptide for their ability to induce NETosis from isolated human neutrophils. Isolated human neutrophils were incubated with 200 nM constructs for 3-4 hr and stained for plasma membrane with Wheat Germ Agglutinin (WGA) labeled with Alexa fluor 488 and anti-double stranded DNA mouse antibody which is cross stained with Alexa fluor 594 labeled anti-mouse secondary antibody. Cells were then visualized using confocal microscopy. All the constructs induced NETs in isolated PMNs except mB Box-97, BBox-87 and ABox. This finding is confirmed by the plate-based assay where released DNA was stained with cell impermeable fluorescence dye SYTOX green (FIG. 1B). Applicant also tested if any of these constructs had any effect on the PMA-induced NETOsis. For that isolated neutrophils were treated with PMA alone or with PMA and various constructs for 3-4 hrs, stained for DNA and plasma membrane and observed using confocal microscopy. Out of 6 constructs tested, only mB Box-97 showed inhibition of PMA-induced NETosis. Upon quantification of NETs % of total cells in different treatment conditions, only mB Box-97 showed significant inhibition of PMA-induced NETosis (FIG. 1C).

### mB Box-97 Inhibits PMA and LPS-Induced NETs Formation and the Release of NETs Associated Proteins.

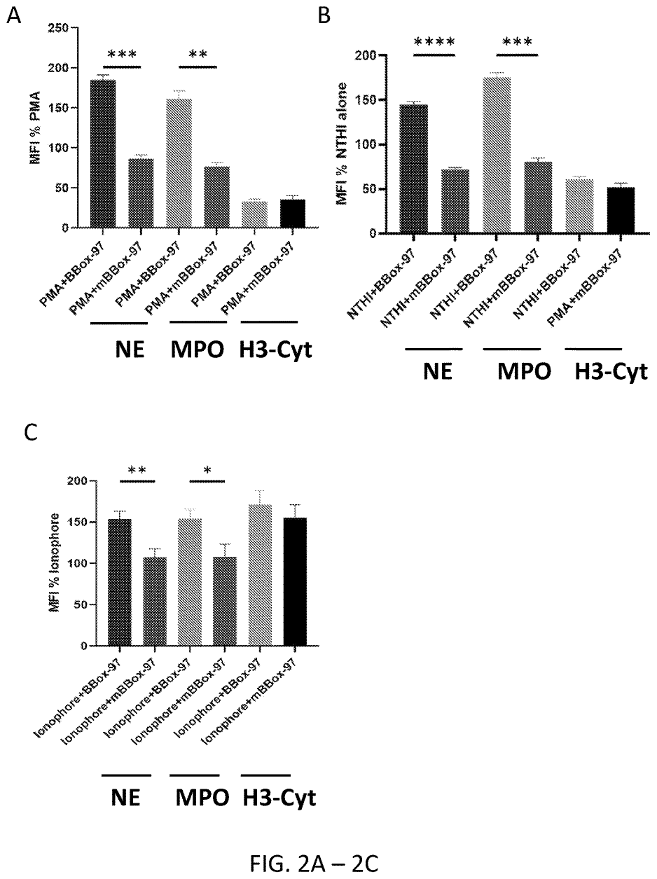

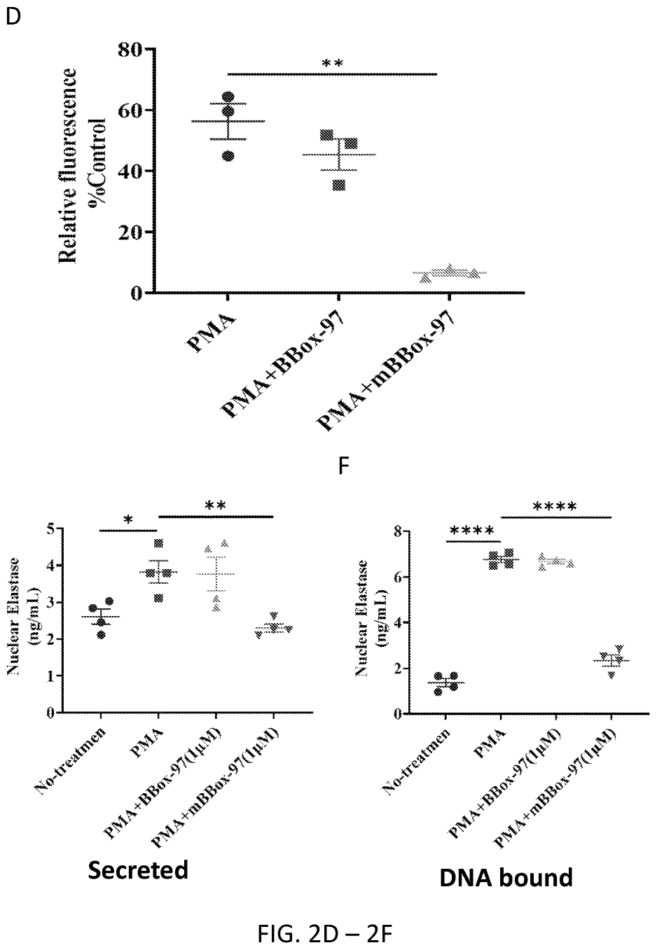

PMA activates protein kinase C (PKC) directly while LPS binds to the respective cell receptor leading to the activation of PKC. Activated PKC phosphorylates the component proteins of nicotinamide adenine dinucleotide phosphate (NADPH) oxidase (NOX) assembly, leading to its activation. Activated NOX generates Reactive Oxygen Species (ROS) which in turn activate a cascade of events leading to the decondensation of chromatin and release of NETs. DNA and protein markers released during this process can be visualized or quantified using specific antibodies. Applicant investigated if inhibition of NETs using mB Box-97 impacts the release of NET-associated proteins like NE and MPO. Isolated human neutrophils (2*10⁵) were treated with PMA (100 nM, 3.5 hrs) or heat-inactivated nontypeable Haemophilus influenzae (NTHI) (1*10⁶, 12-16 hrs) with or without BBox-97 or mB Box-97 (200 nM). NETs were stained for plasma membrane (WGA labeled with Alexa fluor-488), ads-DNA (Alexa fluor-594), and protein markers for NETs-neutrophil Elastase (NE), mitogen Peroxidase (MPO) or citrullinated histone (H3-Cyt) (Alexa fluor-405). The effect of treatments on the formation of NETs was observed by taking z-stack images using confocal microscopy. mB Box-97 inhibited PMA-induced as well as NTHI-induced NETosis, while B Box97 did not affect either. Applicant also calculated the mean fluorescence intensity (MFI) for NE, MPO, and H3-Cyt (FIGS. 2A-2C). MFI for NE and MPO in the mB Box-97 treatment condition was significantly lower than those treated with PMA alone or PMA with BBox-97. There was no difference in the MFI for citrullinated histone observed in either treatment condition (FIGS. 2A-2C).

To quantify the NETosis, Applicant used cell impermeant fluorescent DNA stain SYTOX Green. Isolated neutrophils (5*10³) were treated with PMA with or without mB Box-97 and BBox-97 in 96 well plate for 3.5 hrs, media alone was used as a control. Released extracellular DNA was stained with SYTOX green (1 μM) for 10 mi, washed with PBS, and fluorescence was measured with a fluorimeter. mB Box 97 showed significant inhibition of PMA-induced NET formation whereas BBox had no significant effect (FIG. 2D). Applicant also investigated the release of Nuclear Elastase (NE) in the media and bound to DNA post-PMA-induced NETosis. The quantification was performed using a human NE-specific ELISA kit. Neutrophils were allowed to form NETs in six-well plates using PMA in the presence or absence of mB Box-97 or BBox-97 or no treatment for 3-4 hrs. Media was collected and NETs formed were treated with DNAs to isolate DNA-bound NE. NE released in media and bound to DNA was measured by ELISA. mB Box-97 treatment significantly reduced the release of NE in the media (secreted) (FIG. 2E) as well as NE bound to DNA (DNA bound) (FIG. 2F).

### mB Box 97 Partially Inhibits Ca²⁺ Mediated NETosis.

PMA and LPS-mediated NETosis varies with Ca²⁺ ionophore A23187-induced NETosis. PMA-induced NETosis does not need PAD4 but the same is needed for A23187-mediated NETs formation. NETs formed upon efflux of calcium induced by A23187 involve activation of PAD4 which citrullinates histones and leads to the uncoiling of chromosomes. This pathway mostly bypasses PKC-NOX2-ROS mediated NETosis. Therefore, Applicant investigated the effect of mB Box-97 on the Ca²⁺ ionophore mediated NETosis. Isolated PMNs were treated with A23187 for 4-6 hrs either with mB Box-97 or BBox-97. NETs were visualized with confocal microscopy. Applicant stained for NET-associated marker proteins NE, MPO, or citrullinated histone H3. mB Box-97 inhibited Ca²⁺ ionophore-induced NETosis but the inhibition was not as significant as observed with PMA/LPS-mediated NETOsis. MFI for citrullinated histone shows that mB Box-97 did not have any effect on the histone citrullination in Ca2+ induced NETosis, a similar effect was observed in the PMA/LPS mediated NETosis. The visual inspection as well as the MFI value suggested an increase in the H3-cyt staining upon induction with A23187 (FIG. 2C). Albeit partially but mB Box-97 can inhibit Ca²⁺ ionophore-induced NETosis. This might be because a rise in Ca²⁺ can activate PKC. These observations may indicate that inhibition of NETosis by mB Box-97 might be mediated by the PKC/NOX-ROS pathway rather than the Ca²⁺-PAD4 pathway.

ROS Production in Activated Human Neutrophils is Modulated by mB Box-97 Through Inhibition of p47^{phox} Phosphorylation

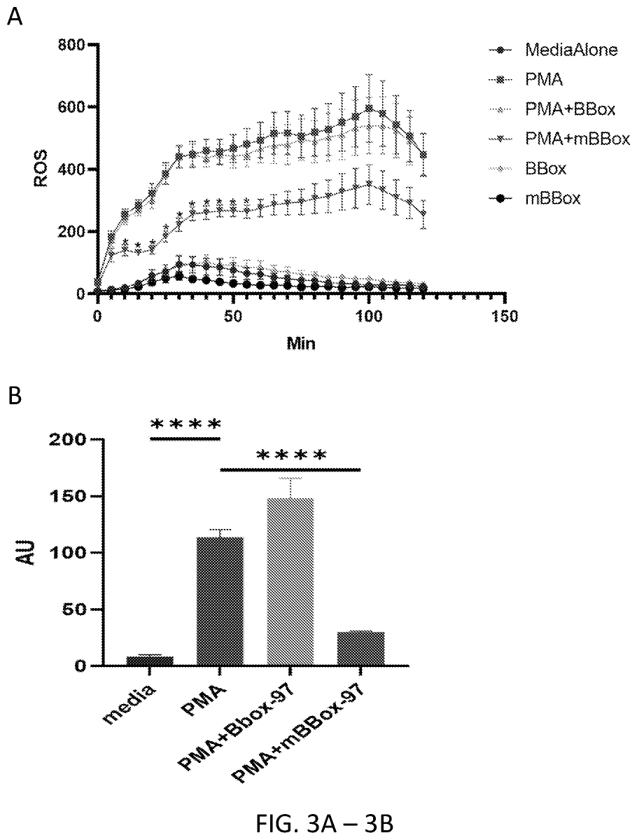

PMA, a diacylglycerol mimetic activates PKC which phosphorylates key components of NOX complex like p47^{phox}, p67^{phox}, and p40^{phox}. Upon phosphorylation, these component proteins assemble in an active NOX complex which generates ROS. ROS production is important for microbial killing as well as the disintegration of nuclear and granular membranes which ultimately finalize the NETosis. Given the importance of ROS in NETosis, Applicant tested the effect of mB Box-97 on the ROS production by neutrophils. Isolated neutrophils were preincubated with Luminol, a chemiluminescent indicator of ROS. Cells were then activated with different treatment conditions and ROS was measured over time for 2 hours with reading at 5 min time intervals. mB Box-97 significantly inhibited PMA-induced ROS production in human neutrophils (FIG. 3A). ROS levels are the indicator of the activity of NOX which is dependent on the phosphorylation of its component proteins therefore Applicant investigated the phosphorylation of a key NOX component protein p47^{phox} for the phosphorylation of Serin 370. Phosphorylation of this residue is shown to be critical for the assembly of the active NOX complex. Proteins from treated neutrophils were separated by SDS-PAGE and probed for phosphorylation of p47^{phox} on ser-370 with phosphor ser-370 specific antibody using western blotting. Total p47^{phox} and GAPDH were used as a control. After densitometric analysis, it was clear that mB Box-97 significantly inhibits the phosphorylation of p47^{phox} while its nonmutant counterpart BBox-97 does not have any effect on the same (FIG. 3B).

### Inhibition of NETosis, ROS Generation, and Release of Antimicrobials by mB Box-97 can Modulate Bacterial Killing by PMNs

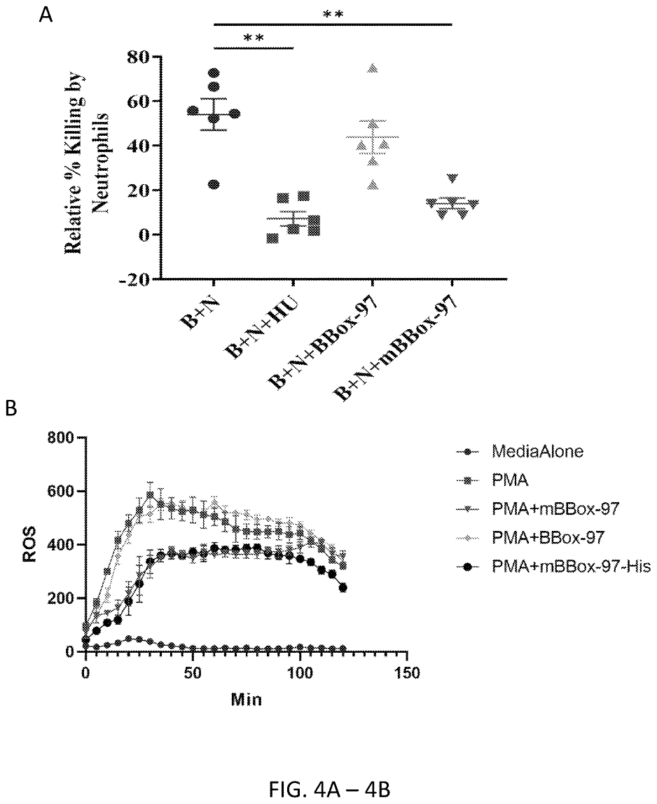

ROS produced by neutrophils contributes to microbial killing besides triggering the NETs formation. Applicant previously showed that inhibition of NETs by bacterial DNABII protein HU from NTHI (HU_{NTHI}) mitigated bacterial killing by neutrophils. As mB Box-97 was able to inhibit NETOsis, ROS production, and release of antimicrobial protein like NE, Applicant tested if it could show any effect on bacterial killing. Towards that goal, Applicant incubated neutrophils with buffer alone, mB Box-97, Bbox-97 and HU_{NTHI} for 4 hr with a 16-hour-old NTHI biofilm. Neutrophils were lysed with Triton X-100 to enable the recovery of viable intracellular bacteria, and total CFU NTHI was computed for each condition to measure the relative percent bacterial killing compared to buffer alone control within the system attributable to PMN NETosis. mB Box-97 inhibited the bacterial killing by neutrophils as compared to BBox-97 and this inhibition was at par with the inhibition by HU_{NTHI} which was used as a positive control (FIG. 4A).

### mB Box-97 Localizes to the Cytoplasm and Plasma Membrane of Human Neutrophils.

Inhibition of NETosis by mB Box-97 was evident with various observations Applicant had but, the specific target of the peptide was not. To gain more insight into the target, Applicant localize the peptide in neutrophils. To achieve that, Applicant synthesized an N-terminal His-tagged recombinant mB Box-97 (mB Box-97-His) peptide. Applicant tested the peptide for its ability to inhibit NETosis and to inhibit ROS production (FIG. 4B). After confirming the NETs inhibitory ability of his tagged mB Box-97 Applicant performed a time-course study to identify the subcellular localization of the peptide. Neutrophils were incubated with his tagged mB Box-97 for 10, 30, 60, 120, or 180 min, fixed with 10% formalin and probed for the peptide with anti-his rabbit primary which was then detected with Alexa fluor-405 labeled goat anti-rabbit secondary. DNA was detected with mouse anti-double-stranded DNA primary which was detected with goat Alexa fluor-595 tagged anti-mouse secondary while the plasma membrane was stained with WGA labeled with Alexa fluor 488. The images were captured using confocal microscopy. Applicant observed that the signal for mB Box-97 remains strong in most of the neutrophils till 120 min post-treatment. Most of the peptide was localized to the cell cytoplasm and plasma membrane. On 180 min some of the peptides appeared to localize at the nucleus.

### Protein Kinase C is Target of mB Box-97.

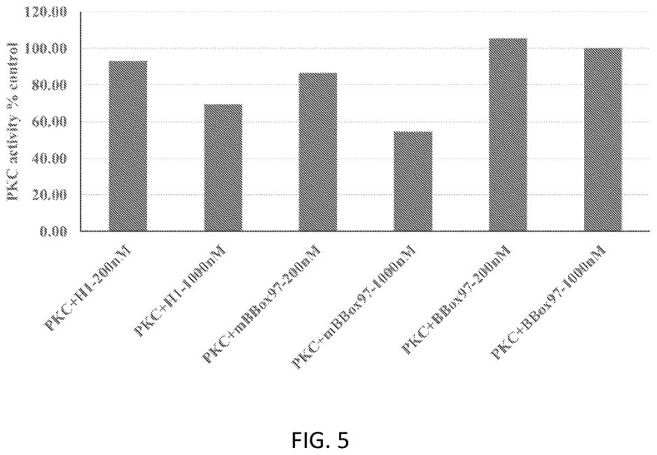

PKC is a key kinase in NETosis whose inhibition leads to the inhibition of NETosis. PKC is involved in the phosphorylation of and thereby assembly of active NOX complex. Based on the observations of the cytoplasmic localization of mB Box-97, and inhibition of p47^{phox} phosphorylation Applicant investigated if mB Box-97 has any effect on PKC activity. Toward that goal, Applicant used the PKC activity kit. mB Box-97 in 2 different concentrations was incubated with PKC for 10 min and then the mix of the two was incubated with the substrate. (FIG. 5).

The formation of NETs is an important function of the active immune system. Studies showing the importance of NETs in the remedy of various pathologies are gathering steam as do studies showing the negative impact of excessive NET formation. Essentially, the NETs formation must be retained at a balance of its production and clearance. In vitro, various upstream stimulators, including LPS, TNF, IL-8, and PKC agonists, and some pre-inflammatory molecules like HMGB1 can activate NET formation. During NETosis various bioactive molecules get released leading to microbial killing. However, due to their nonspecific nature, components of NETs can cause injury to surrounding tissues by themselves or by increasing the pro-inflammatory response. They can also play a role in the augmentation of the inflammation seen in autoimmune diseases such as psoriasis, rheumatoid arthritis (RA), and systemic lupus erythematosus (SLE). In addition, autoinflammatory diseases like gout have been associated with NETosis. Excessive NETs can also cause a physical barrier in blood flow leading to atherosclerosis and/or stroke. Cancer cells can increase NETosis by priming platelets in pancreatic cancer. NET-mediated platelet activation can promote several negative outcomes linked with late-stage metastatic breast cancer, including venous thromboembolism (VTE).

Recent studies on COVID-19 showed that there is a significant role of enhanced neutrophil infiltration and the release of NETs, complement activation and vascular thrombosis during necroinflammation in COVID-19. Formation of NETs in microvessels increases the inflammatory response and vascular micro-thrombosis, which in the lungs of patients leads to ARDS. The serum level of NETosis markers was elevated in the patients under intensive care, and mechanical ventilation than in the patient's breathing room air which suggests NETosis may also be associated with disease severity in COVID-19. Inhibition of NETs may reduce the severity of many such diseases thereby improving survival. Herein, Applicant describe an HMGB1-based mutant peptide mB Box-97 which inhibits the formation of NETs induced by different stimuli.

There are multiple direct or indirect inhibitors of NETs formation in preclinical studies though none of them are approved for the treatment of NETs-related complications. Some of these are molecules that have been in use in clinics for many years like Hydroxychloroquine, Methotrexate, and Prednisolone (active metabolite of prednisone). Some are humanized antibodies e.g., rituximab (anti-CD20 mAb), belimumab (fully human IgG1λ recombinant mAB), and Tocilizumab (mAb against IL-6 receptor Others are molecules that can inhibit the function of NE or MPO or PAD4). Most of these act as indirect inhibitors of NETosis. Though multiple inhibitors are being tested there is still a need for efficient inhibitors which will not ignite unwanted side effects and hamper normal functions of the neutrophils other than NETosis.

In a previous study, Applicant showed that mutated HMGB1 loses its proinflammatory activity while retaining its anti-biofilm activity (Devaraj A. et al., 2021, J Clin Invest 131(16)). In the present disclosure, Applicant constructed a mutated peptide mB Box-97 based on HMGB1 which retained its anti-biofilm ability against multiple microbes without showing any pro-inflammatory activity. Applicant observed that this peptide can inhibit NETs formation induced using PMA and LPS while showing partial inhibition when tested against Ca²⁺-induced NET formation. The pathways activated by these stimuli are different and hence the outcome of the inhibition using mB Box-97. PMA and LPS-mediated NETosis often involve activation of PKC leading to the phosphorylation of key components of NADPH oxidase which ultimately helps in the formation of NOX complex and generation of ROS. Applicant studied the effect of mB Box-97 on the phosphorylation of p47^{phox} and found that its phosphorylation was inhibited by the peptide (FIG. 3B). Applicant also observed the inhibition of ROS generation which was in line with the inhibition of the p47^{phox} phosphorylation. ROS generation is important for the release of antimicrobials from the granulocytes and also the release of decondensed chromatin. ROS triggers the dissociation of NE from a membrane-associated complex into the cytosol and activates its proteolytic activity in a myeloperoxidase (MPO)-dependent manner. Activated NE moves to the nucleus cleaving chromatin and releasing it into the cytoplasm thereby finalizing the NETs formation. Applicant observed a reduction in the release of NE from neutrophils upon treatment with the mB Box-97 (FIG. 2E). Formation of NET, release of antimicrobials, and generation of ROS all affect the microbial killing by NETs. As mB Box-97 could inhibit all of these, Applicant tested its effect on microbial killing and found that it was able to inhibit neutrophil-mediated bacterial killing (FIG. 4A).

PKC is a key kinase in the process of NETosis and its inhibition impaired NETosis. Most of the evidence in this study pointed us toward the involvement of mB Box-97 in the inhibition of PKC activity. For example, inhibition of p47phox phosphorylation (FIG. 3B), and localization of the peptide in the cytoplasm as well as plasma membrane. Therefore, Applicant investigated the effect of mB Box-97 on PKC activity using a standard PKC assay kit and found that mB Box-97 inhibits PKC activity (FIG. 5). Partial inhibition by mB Box-97 of Ca²⁺ mediated NETosis can be attributed to the inhibition of PKC as an influx of Ca²⁺ can also activate PKC besides the activation of PAD4.

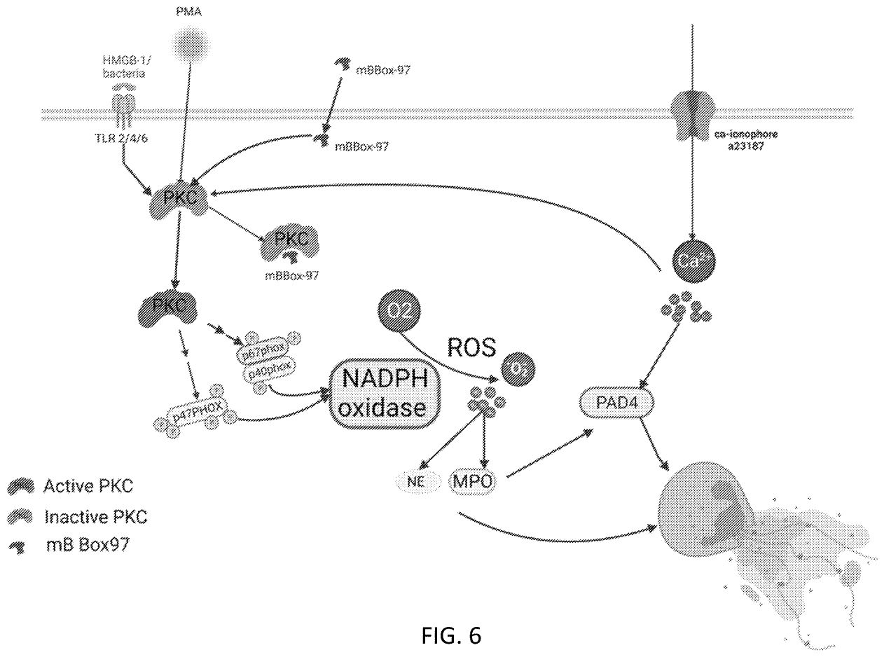

Without being bound by a particular theory, it is believed that HMGB1-based peptide mB Box-97 inhibits the activity of Protein Kinase C leading to the inhibition of phosphorylation of key NADPH oxidase component proteins. This inhibits the active assembly of NOX and reduces ROS production. Reduced ROS production hampers the release of granulocyte-associated proteins like NE and ultimately production of NETs (FIG. 6). mB Box-97 does not induce inflammation while retaining the ability to inhibit the formation of biofilms. mB Box-97 has excellent therapeutic potential.

### Treating Rheumatoid Arthritis Using mB Box-97.

Abnormal, excessive NETosis is believed to be an underlying reason for autoimmune diseases such as rheumatoid arthritis. In order to show that subjects suffering from rheumatoid arthritis (RA) will benefit from inhibiting abnormal, excessive NETosis using the mB Box-97 peptide, preclinical animal models of RA will be used. Exemplary animal models of RA are found in Meehan, Gavin R., et al. (Annals of the Rheumatic Diseases 80.10 (2021): 1268-1277) and Zhao, Ting, et al. (Frontiers in Immunology 13 (2022): 887460), which are incorporated herein in their entireties. In some embodiments, the animal model of RA is a rodent model, or a non-human primate model. In some embodiments, the animal model of RA is selected from a collagen-induced arthritis (CIA) model, an adjuvant-induced arthritis (AA) model, a delayed-type hypersensitivity arthritis (DTHA) model, an anticitrullinated peptides antibodies (ACPA)-mediated arthritis model, or a spontaneous model.

An animal model of RA is treated with between 50 nM and 2 μM mB Box-97 peptide as described herein, and diagnostic blood markers of RA (e.g., C-reactive protein (CRP), Rheumatoid factor (RF), or anti-cyclic citrullinated peptide (anti-CCP)) are measured regularly (e.g., once every 3 hours, once every 6 hours, once every 12 hours, once every 24 hours, once every other day, once every three days, once every four days, once every five days, once every six days, or once every week). A reduction in diagnostic blood markers of RA is observed over time in animals treated with mB Box-97 as compared to controls who were administered a scrambled control peptide. Also a reduction in RA symptoms (e.g., swollen joints, accumulation of fluid in the ankles, joint pain, joint inflammation) is also observed upon mB Box-97 as compared to controls who were administered a scrambled control peptide.

### Example 2: MB Box-97 Disrupts and Prevents Biofilm Formation by Diverse Human Pathogens

HMGB1-Derived Peptides that Contain the B-Box and mB Box-97 Retain In Vitro Biofilm Disruption Activity

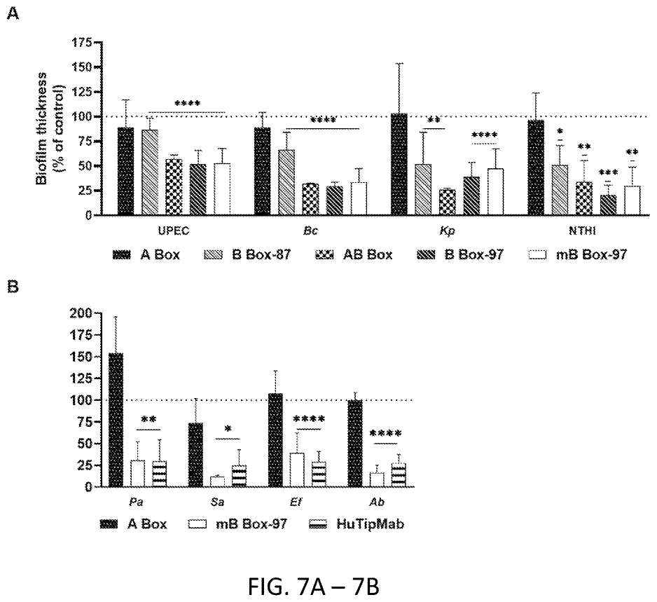

First, Applicant tested disruptive abilities of HMGB1-derived peptides. Addition of A Box to established biofilms (UPEC, B. cenocepacia, NTHI or K. pneumoniae) had no significant effect on measured biofilm parameters (FIG. 7A). While B Box-87 had only limited anti-biofilm activity, the A-B Boxes and B Box-97 retained anti-biofilm activity (FIG. 7A). Since only the B Box can modulate DNA bending, Applicant hypothesize that HMGB1 disrupts biofilms, at least in part, via DNA-binding/bending. As the B Box is reported to contain pro-inflammatory activity, mediated primarily through interactions with TLR4-MD2 that are dependent on residue C106, Applicant created a modified recombinant B Box-97 variant (mB Box-97) with a C106S mutation (FIG. 1A). The mB Box-97 variant disrupted biofilms formed by UPEC, NTHI, B. cenocepacia and K. pneumoniae in vitro equivalent to that induced by B Box-97.

### mB Box-97 Disrupts Biofilms In Vivo

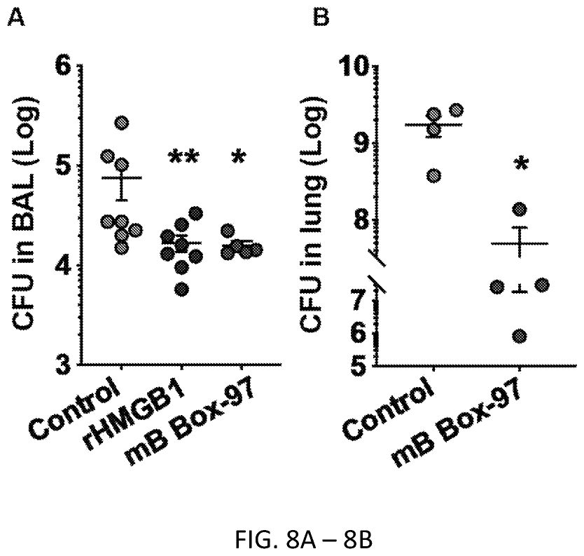

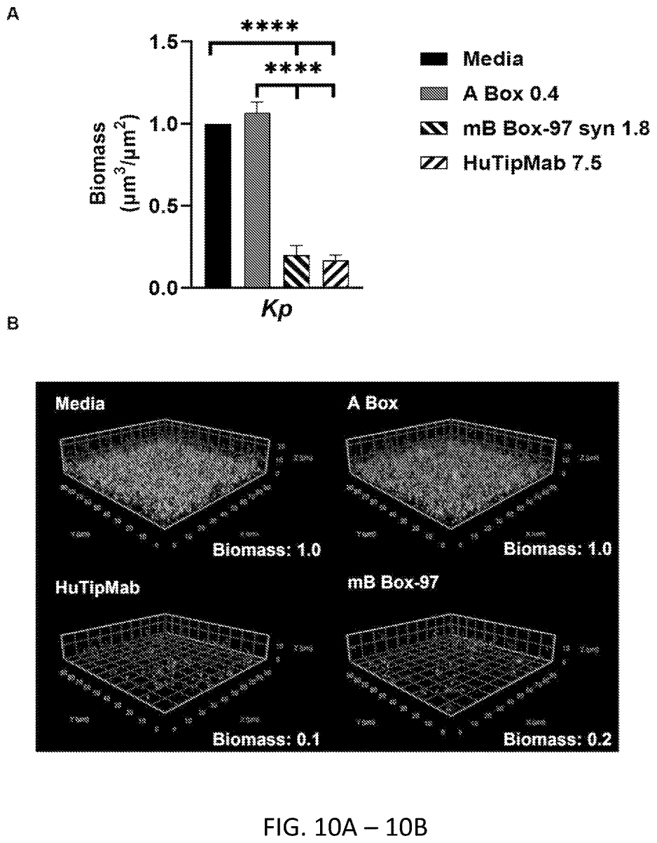

To determine whether mB Box-97 could prevent lung infection and also limit inflammation as was intended, Applicant challenged adult C57BL/6 mice with 10⁷ (FIG. 8A) or 10⁸ (FIG. 8B) CFU of B. cenocepacia (Bc) intratracheally (i.t.), along with either 200 nM of mB Box-97 or a negative control. After 18h, mice were euthanized for collection of both BAL fluids and lung tissue. Lung tissue was homogenized, diluted and plated to determine relative CFUs, whereas BAL fluids were similarly diluted and plated. Mice administered either rHMGB1 or mB Box-97 contained significantly fewer Bc in both BAL and lung tissue compared to control mice, which suggested that mB Box-97 inhibited aggregate biofilm formation in the murine airways even at high bacterial challenge dose (10⁸ CFU) and further, that this approach facilitated bacterial clearance which thereby suggested preventative efficacy. In addition, no mice displayed signs of sepsis that required euthanasia prior to the study endpoint despite use of potent therapeutic doses of mB Box-97. While LPS alone induced over 100 μg/ml of TNF-α [a gold standard surrogate for sepsis induction], neither rHMGB1 nor mB Box-97 elicited detectable TNF-α, nor did they induce additional pro-inflammatory signaling when administered to LPS-primed mice. Here, Applicant demonstrated that mB Box-97 retained its anti-biofilm activity without inducing inflammation by both limiting the bacterial load and/or preventing biofilm aggregates from forming in the murine lung as well as not inducing TNF-α.

### Synthetic mB Box-97 (mB Box-97_{syn}) is Equivalent to Recombinant mB Box-97 In Vitro

Native HMGB1 is heavily post translationally modified with these modifications affecting various HMGB1 functions. Indeed, even recombinant proteins expressed in bacteria often have posttranslational modifications. To determine if these modifications play a role in anti-biofilm disruption, Applicant synthesized an otherwise identical 97 amino acid peptide based on the coding sequence of the recombinant mB Box-97 which will henceforth referred to as mB Box-97_{syn}. To determine if mB Box-97_{syn} retained DNA binding activity, Applicant performed an electromobility shift assay using a Holiday Junction DNA substrate that mimics the cross strands of the eDNA lattice of bacterial biofilms and is also a natural substrate of HMGB1 and compared the ability of all mB Box-97_{syn} against the various truncations of HMGB1. Recombinant versions of all the truncations that contained complete domain structures including mB Box-97 and mB Box-97_{syn} were similarly able to bind to Holliday junction DNA. As an additional test of stability. recombinant mB Box-97 and mB Box-97_{syn} were incubated in human serum and showed similar stability consistent with the fact that any posttranslational modifications present on the recombinant mB Box-97 failed to affect protein stability. As a final test, Applicant also showed that mB Box-97_{syn} significantly disrupted four additional high priority ESKAPEE pathogens regardless of biofilm age (FIG. 7B). Here, Applicant also utilized a humanized monoclonal antibody directed against the binding tips of the DNABII proteins (‘HuTipMab’, previously shown to have broad biofilm disruption activity) as a positive control (FIG. 7B). Biofilms that had formed for 24 h then treated for 2 h with either 1.2 μg of mB Box-97 or 5.0 μg of HuTipMab were significantly disrupted (P. aeruginosa; 30.5%, 29.8%, S. aureus; 12.2%, 24.8%, E. faecium; 39.2%, 29.1%, A. baumannii; 16.7%, 27.8% (P=0.03-P<0.0001) compared to treatment with media alone.

To determine whether older biofilm age might impact the ability of mB Box-97_{syn} to disrupt a bacterial biofilm, as these biofilms contain an increasingly greater concentration of eDNA, Applicant tested this hypothesis using biofilms formed by the respiratory tract pathogen NTHI. To this end, Applicant treated 48 h and 72 h NTHI biofilms with either 1.2 μg of mB Box-97 or 5.0 μg of the murine monoclonal antibody (MsTipMab) directed against the same epitopes as HuTipMab. Whereas these older NTHI biofilms were not disrupted by incubation with a murine monoclonal antibody directed against a non-protective domain of a bacterial DNABII protein (MsTailMab), they were significantly disrupted by both mB Box-97 and MsTipMab (48h; 47.3% and 47.2%. 72 h; 46.4% and 34.4%), compared to media alone. Biofilms grown for 96 h required twice the aforementioned concentrations of both mB Box-97_{syn} or MsTipMab for similar disruption (e.g., 49% and 37%, respectively), as expected given that eDNA and DNABII concentration increases with biofilm maturity. These results were statistically significant (P=0.02, P=0.005, respectively). Applicant henceforth used only mB Box-97_{syn} for the remainder of the experiments.

### mB Box-97_{syn} Prevents Biofilm Formation In Vitro

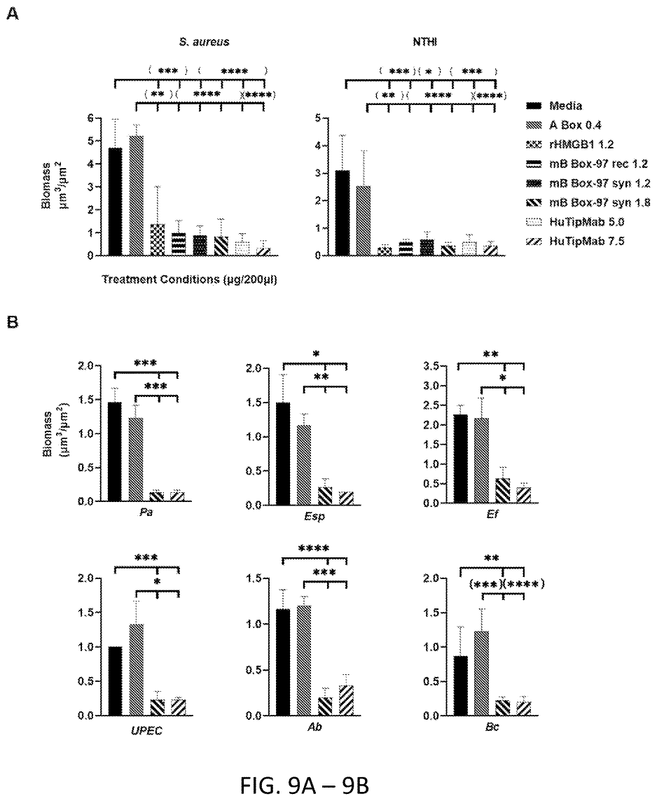

Applicant now hypothesized that mB Box-97 could also prevent the formation of bacterial biofilms as this hypothesis is consistent with the domain variant's now demonstrated ability to disrupt established biofilms in vitro. To test this hypothesis, Applicant first tested the ability of mB Box-97 to prevent NTHI and S. aureus, a gram-negative and gram-positive pathogen, respectively from forming a biofilm by use of the same two concentrations of mB Box-97 as those used earlier for in vitro disruption assays. Incorporation of rHMGB1 and mB Box-97_{rec} served as positive controls, with media-only and A Box used as negative controls. Applicant also now wanted to test two concentrations HuTipMab, previously shown to have broad biofilm disruption activity, for its potential to also perhaps prevent biofilm formation. After 16 h of incubation, both positive controls and all tested concentrations of mB Box 97 or HuTipMab significantly inhibited biofilm growth by both NTHI and S. aureus compared to negative controls (FIG. 9A) (P=0.01-P<0.0001). The highest tested concentrations of mB Box-97 and HuTipMab (e.g., 1.8 μg and 7.5 μg, respectively) limited growth to that of a monolayer of bacteria or less, (Biomass <1.0 μm³/μm², P<0.0001) with the absence of any characteristic 3D biofilm architecture.

Next, Applicant began to assay the breadth of ability of mB Box-97 and/or HuTipMab to prevent biofilm formation. To do so, Applicant used the most effective concentration of mB Box-97 or HuTipMab in prevention of biofilm growth by NTHI and S. aureus to similarly prevent biofilm formation by the remaining high priority ESKAPEE pathogens or B. cenocepacia (FIG. 9B). Applicant observed significant biofilm prevention after 16 h incubation of either P. aeruginosa, Enterobacteriacea sp, E. faecium, uropathogenic E. coli, A. baumannii or B. cenocepacia with either mB Box-97 (P=0.03-P<0.0001) or HuTipMab (P=0.02P<0.0001) compared to negative controls. To provide additional evidence of the preventative activity observed, data generated with the pathogen K. pneumoniae (FIG. 10A) are presented along with corresponding representative CSLM images (FIG. 10B). These differences were statistically significant (P=0.03-P<0.0001).

mB Box-97_{syn} Synergizes with a Humanized Monoclonal Antibody Directed Against Protective Domains of a DNABII Protein to Prevent Biofilm Formation

As shown in FIGS. 4&5, both mB Box-97 and HuTipMab significantly prevented biofilm formation by 9 pathogens including the 7 high priority ESKAPEEs when tested individually. Here, Applicant now assessed whether mB Box-97 and HuTipMab's could act either additively or perhaps synergistically to prevent biofilm formation. To assess this, Applicant again used NTHI and S. aureus as model pathogens. Applicant first incubated NTHI or S. aureus with serial 1:2 dilutions of the maximal dose of either mB Box97 or HuTipMab used in earlier prevention assays to determine relative preventative activity.

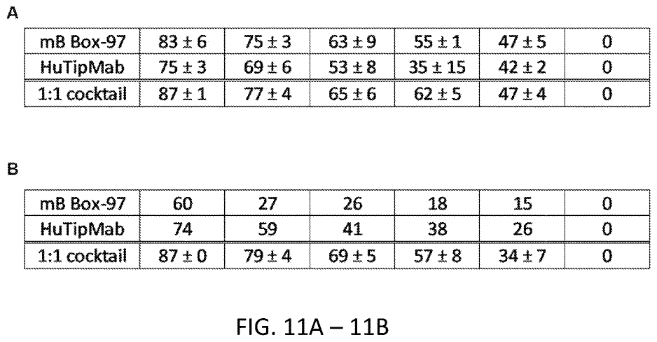

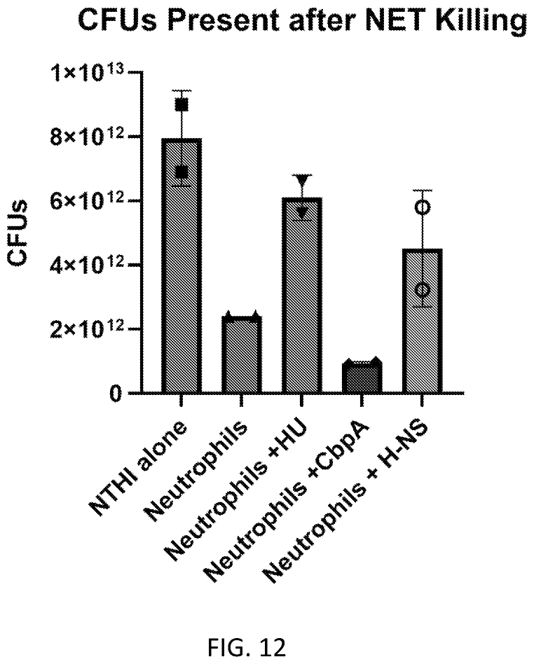

As shown, prevention of NTHI biofilms by dilutions of HuTipMab (top row, FIG. 11A) ranged from 0-72%, whereas prevention by dilutions of mB Box 97 (final column, FIG. 11A) ranged from 0-43%. For S. aureus prevention by dilutions of HuTipMab (top row, FIG. 11B) similarly ranged from 0-74%, whereas prevention by dilutions of mB Box97 (final column, FIG. 11B) ranged from 0-60%. To assess potential additive or synergistic prevention activity of these two biologicals, Applicant then incubated NTHI or S. aureus with a cocktail of mB Box-97 plus HuTipMab at multiple similar 1:2 fold decreasing dilutions for 16 h and determined relative biomasses (see values within diagonal boxes within both panels of FIG. 11). For NTHI (Panel A, diagonal boxes), relative percent prevention values exceeded that of either individual component for cocktails comprised of admixed full doses to those that contained 1/16^{th} doses of each. When each biological was used at ⅛^{th} dose, the outcome was synergistic (66% combined compared to 20% or 5% individually). For S. aureus (Panel B, diagonal boxes), relative percent prevention values again exceeded those of either individual component alone for cocktails comprised of admixed full doses to those that contained ⅛^{th} doses of each. Whereas mathematically, no distinct synergistic outcome was noted, there was additive prevention when Applicant assessed prevention using cocktails of each biological at ¼^{th} and ⅛^{th} doses each (69% compared to 41% or 26% individually; 57%, compared to 38% or 18% individually, respectively).

Applicant saw that in combination and tested against both pathogens, mB Box-97 and HuTipMab yielded greater prevention at sub-maximal tested concentrations (½ max concentration of both treatments prevented 75% of NTHI biofilm compared to negative control and 2 max concentration of both treatments prevented 79% S. aureus biofilm compared to negative control) as compared to prevention of the highest tested concentrations of either treatment administered separately (max concentration of HuTipMab tested prevented NTHI biofilm growth by 72%, and max concentration of mB Box-97 prevented NTHI biofilm growth by 43% compared to negative control. Max concentration of HuTipMab tested prevented S. aureus biofilm growth by 74%, and max concentration of mB Box-97 prevented S. aureus biofilm growth by 60% compared to negative control).

Applicant also determined combination index

$$( CI , Combination Index ( CI ) = \frac{[ Comp 1 IC 50 in combo ]}{[ Comp 1 IC 50 alone} + \frac{[ Comp 2 IC 50 in combo ]}{[ Comp 2 IC 50 alone}$$

scores, a calculation used to assess overall synergy between two or more compounds. Scores obtained for NTHI and S. aureus were 0.4 and 0.5, respectively, which indicated that mB Box-97 and HuTipMab acted synergistically when used in combination given that CI scores <1 indicate synergy.

### Materials and Methods

Humanized Monoclonal Antibody Against a Tip Chimer Peptide Designed to Mimic the Immunoprotective Domains of the DNABII Protein Integration Host Factor (IHF).

Humanized monoclonal antibody (of the IgG isotype) against the tip-chimer peptide (HuTipMab) was engineered from a murine monoclonal antibody.

Synthetic mB Box-97. (mB Box-97_{syn}; LifeTein®, LLC; for ease of large-scale production and > to 95% homogeneity)

Bacterial species and sources. NTHI strain 86-028NP was isolated from the nasopharynx of a child with chronic otitis media at Nationwide Children's Hospital. Enterobacter spp. and K. pneumoniae were isolated from. S. aureus strain 29213, A. baumanii strain 17978, and P. aeruginosa strain 27853 were obtained from the ATCC. E. faecium Com12 strain was isolated from feces of healthy human volunteers. Strains not obtained from the ATCC were maintained frozen at low passage number in liquid nitrogen.

Disruption of bacterial biofilms. NTHI and S. aureus were cultured on chocolate agar for 18-24 hours at 37° C. in a humidified atmosphere that contained 5% CO₂. NTHI was then resuspended in brain heart infusion broth supplemented with heme [2 μg/mL] and β-NAD [2 μg/mL](sBHI) broth to an OD of 0.1 at 490 nm. S. aureus was resuspended in brain heart infusion broth (BHI) to an OD₄₉₀ₙₘ of 0.1. The cultures were then diluted in their respective media to contain approximately 2×10⁵ CFU/mL, and 200 μL of this suspension was inoculated into each well of an 8-well chambered cover-glass slide (Thermo Fisher Scientific, Waltham, MA). P. aeruginosa and E. faecium were cultured on tryptic soy agar (TSA) as described above, then suspended in tryptic soy broth (TSB) to an OD₄₉₀ₙₘ of 0.1. The cultures were then diluted in their respective media as described above with 200 μL of this suspension used to was inoculate each well of an 8-well chambered cover-glass slide. After 16 hours of incubation of each of the bacterial species at 37° C. 5% CO₂, medium was replaced with the respective fresh medium and incubated for another 8 hours. At 24 hours, the medium was replaced with the respective fresh medium (control) or fresh medium that contained mB Box-97syn, (1.2 μg/200 μL), A Box or (1.2 μg/200 μL) or HuTipMab (5.0 μg/200 μL) and incubated at 37° C. 5% CO₂ for 2 hours. All biofilms were then washed twice with 1× Dulbecco's phosphate-buffered saline without calcium or magnesium (DPBS) (Corning, Corning, NY) and stained with LIVE/DEAD stain (Thermo Fisher Scientific, Waltham, MA) per manufacturer's instructions. Biofilms were again washed then fixed with 1.6% paraformaldehyde, 0.025% glutaraldehyde, and 4% acetic acid in 0.1 M phosphate buffer at pH 7.4. Biofilms were imaged with a ×63 objective on a Zeiss 800 confocal laser scanning microscope (CLSM; Zeiss) and analyzed with COMSTAT2. Biomass values (μm³/μm²) were calculated by COMSTAT2 and represent the mean of 3 biological replicates±the SEM.

Prevention of bacterial biofilm formation. NTHI and S. aureus were cultured and resuspended as described above then diluted in their respective media to contain ˜5×10³ CFU/mL or ˜1×10³ CFU/mL, respectively, and 200 μL of this suspension was inoculated into each well of an 8-well chambered cover-glass slide. UPEC strain UTI89, B. cenocepacia, K. pneumoniae, Enterobacter spp, and A. baumanii were cultured and resuspended as described above then diluted in LB broth to contain ˜1×10³-1×10⁵ CFU/mL, and 200 μL of this suspension was inoculated into each well of an 8-well chambered cover-glass slide. P. aeruginosa and E. faecium were cultured and suspended as described above then diluted to contain ˜1×10³ CFU/mL, and 200 μL of this suspension was inoculated into each well of an 8-well chambered cover-glass slide. Upon incubation at 37° C. 5% CO₂ for 16 hours, HMGB1 isoform rHMGB1 (1.2 μg/200 μL), A Box (1.2 μg/200 μL) mB Box-97_{rec} (1.2 or 1.8 μg/200 μL), mB Box-97_{syn} (1.2 or 1.8 μg/200 μL) or HuTipMab (5.0 or 7.5 μg/200 μL) was added to respective wells of all pathogens tested. Following incubation, all biofilms were then washed once with DPBS and fixed as described above. Biofilms were imaged and analyzed as described above. All assays were repeated a minimum of 3 times on separate days. Data are presented as mean+/−SEM. HuTipMab antibody is as described in U.S. Pat. No. 11,104,723 and comprises a heavy chain complementarity-determining region 1 (CDRH1) comprising a sequence of GFTFRTY (SEQ ID NO: 47) (aa 50 to aa 56 of SEQ ID NO: 9); a heavy chain complementarity-determining region 2 (CDRH2) comprising a sequence of GSDRRH (SEQ ID NO: 48) (aa 76 to aa 81 of SEQ ID NO: 9); a heavy chain complementarity-determining region 3 (CDRH3) comprising a sequence of VGPYDGYYGEFDY (SEQ ID NO: 49) (aa 121 to aa 133 of SEQ ID NO: 9); a light chain complementarity-determining region 1 (CDRL1) comprising a sequence of QSLLDSDGKTF (SEQ ID NO: 51) (aa 47 to aa 57 of SEQ ID NO: 15); a light chain complementarity-determining region 2 (CDRL2) comprising a sequence of LVS (aa 75 to aa 77 of SEQ ID NO: 15); and a light chain complementarity-determining region 3 (CDRL3) comprising a sequence of WQGTHFP (SEQ ID NO: 52) (aa 114 to aa 120 of SEQ ID NO: 15).

Determination of synergistic effect between mB Box-97 and HuTipMab. NTHI and S. aureus were cultured on chocolate agar as described then resuspended and diluted in their respective media to contain ˜5×10³ or ˜1×10³ CFU/mL respectively, and 200 μL of this suspension was inoculated into each well of an 8-well chambered cover-glass slide. mB Box-97 and HuTipMab were added to these bacterial solutions to yield 0.05-1.8 and 0.23-7.5 μg/200 μL. After 16 hours of incubation at 37° C. 5% CO2, biofilms were washed once with sterile saline and fixed, imaged and analyzed as described above. All in vitro biofilm assays were repeated a minimum of 3 times on separate days. Data are presented as mean+/−SEM.

Statistical analyses. Graphical results and statistical tests were performed with GraphPad Prism 9 for all in vitro assays. Statistical significance for in vitro assays was assessed by 1-way ANOVA with multiple comparisons. P values of less than 0.05 were considered significant.

### Example 3: H-NS Disables Neutrophil Extracellular Traps

H-NS is Released into Bulk Media as the Biofilm Matures

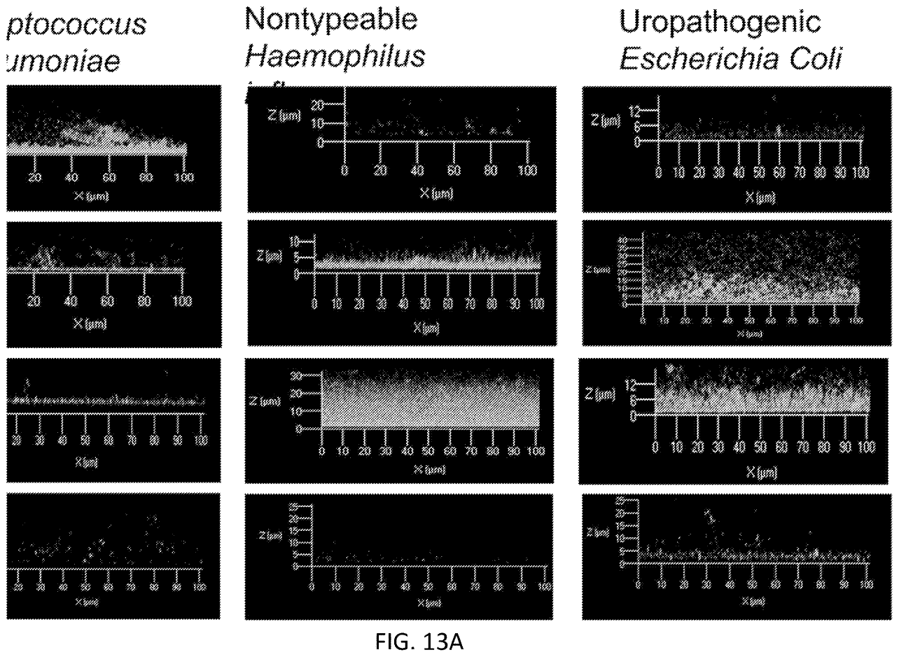

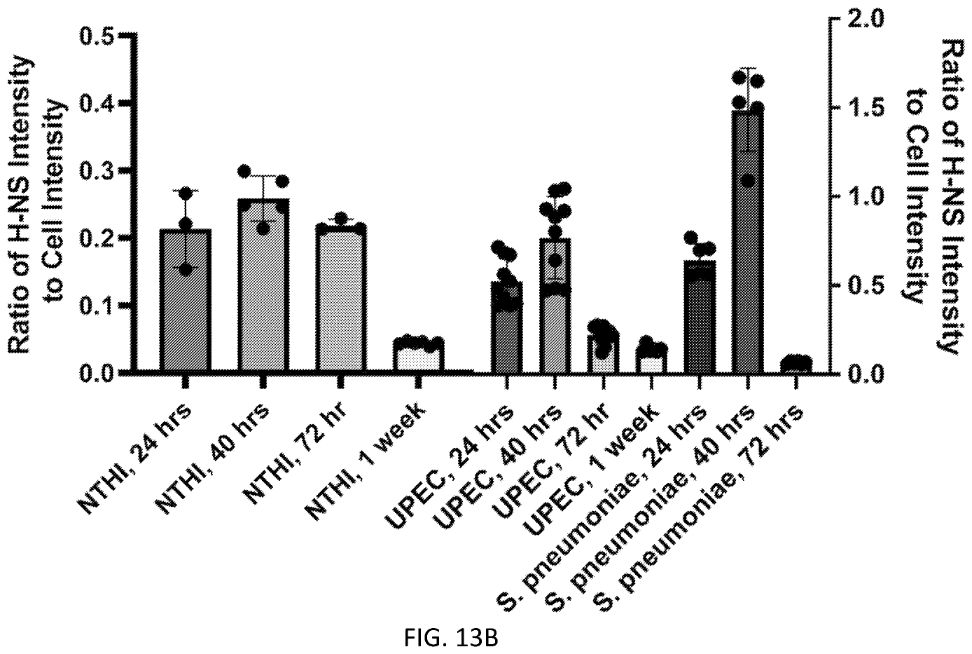

H-NS is a B-DNA binding protein that has been found outside of bacterial cells within a biofilm. When H-NS is depleted within a biofilm it has no impact on the biomass and thickness of said biofilm indicating that H-NS is not necessary for biofilm structure in the way that DNABII proteins are. When the ratio of H-NS fluorescence intensity was measured compared to the fluorescence intensity of cells it was found to increase from 24 hours to 40 hours in the UPEC, NTHI, and S. pneumoniae biofilms, these levels then dropped at 72 hours and continued to decrease as the biofilm reached 1 week of age. In the S. pneumoniae biofilm, the F.I. ratio of H-NS to cells at 1 week was too low to be quantified (FIGS. 13A-13B).

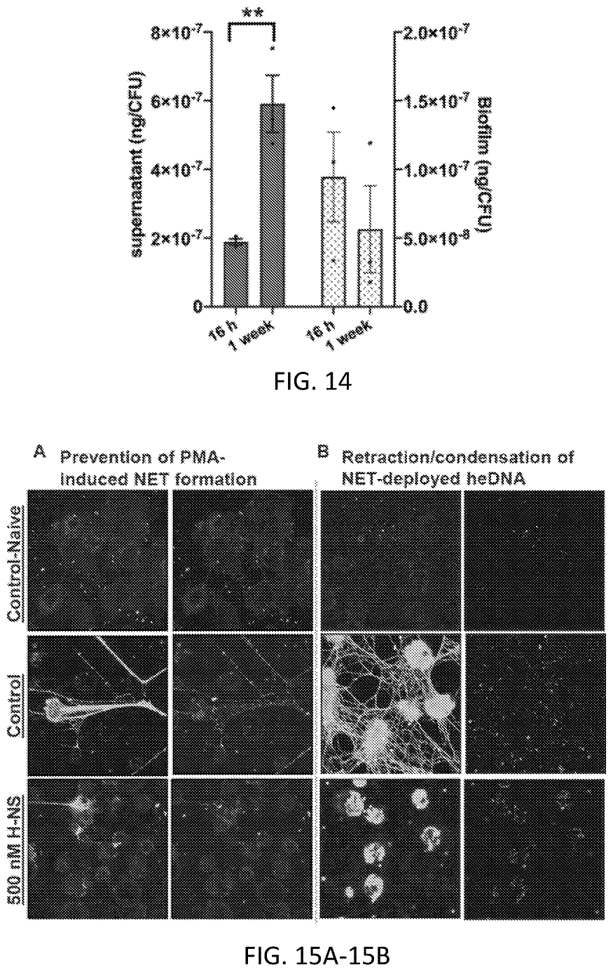

To confirm that H-NS is exiting the biofilm and entering the bulk media, the concentration of H-NS in both the biofilm and media was measured using western blots. Comparing 16 hours and 1 week old biofilms revealed that a shift from the majority of H-NS being present in the biofilms to the media indicating that H-NS levels were diminishing within the biofilm because H-NS was being released as the biofilm matured (FIG. 14).

To further confirm that the levels of H-NS is lowering in the biofilms to enter the bulk media and not due to a change in gene expression, RT-PCR was carried out.

H-NS Prevents Neutrophil DNA Release and Condenses Previously Release NET eDNA

In vivo, NET eDNA is released by neutrophils when stimulated by pathogens, but NETosis can also be stimulated in vitro using PMA. The strands of DNA that exit the cell are due to unwinding of nuclear DNA as histones are citrullinated which can be visualized using confocal microscopy. CbpA, a DnaJ homolog, that preferentially binds to curved DNA was utilized as a negative control given that both H-NS and CbpA bind curved DNA, but CbpA does not condense DNA. H-NS prevented the release of neutrophil DNA while CbpA did not (FIGS. 15A-15B). When neutrophils were stimulated using PMA and NETs formed, H-NS also was able to compact neutrophil eDNA in the case of pre-formed NETs. Further staining of the antimicrobials bound to NET eDNA in addition to the eDNA showed that the antimicrobials were still bound to the eDNA and they weren't displaced by H-NS.

### H-NS Prevents NET Killing of Bacteria

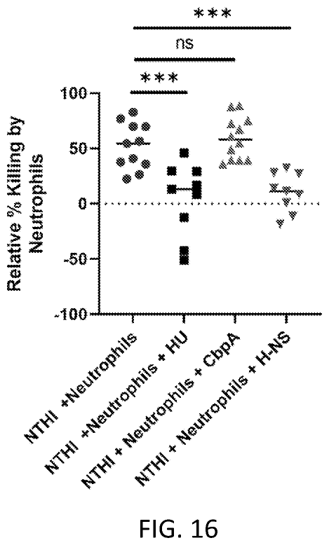

While NETs have limited ability to infiltrate and kill bacteria within biofilms, they are capable of killing free bacteria and can cordon off biofilm growth. With NETs relying on their released DNA tentacles to kill bacteria, and H-NS leading to the condensation of these tentacles, it was likely that H-NS would prevent NET Killing of bacteria in addition to changing NET morphology. HU had been shown to change NET eDNA to Z-DNA and prevent NET killing and was therefore used as a positive control and as a comparison to determine the degree to which H-NS could prevent NET killing of bacteria4. H-NS significantly reduced the amount of bacteria killing due to NETs as did HU (FIG. 16). CbpA was unable to prevent killing of free bacteria by NETs which correlated with the inability of CbpA to change NET morphology and preventing NETosis. The ability of H-NS to prevent NET clearing of free bacteria confirms that by condensing the eDNA of NETs, H-NS possible acts as a defense on behalf of the biofilm against the host immune system.

### Materials and Methods

Bacterial strains and plasmids. NTHI Strain 86-028NP is a clinical isolate recovered from the nasopharynx of a child undergoing tympanostomy tube insertion that was streaked out on chocolate agar and grown overnight in a 37° C. incubator at 5% CO₂. Uropathogenic E. coli (UPEC) (SG1019) was a clinical isolate from a febrile UTI, renal abscess clinical isolate that was streaked out and incubated overnight at 37° C. with 5% CO₂. Streptococcus pneumoniae 1121 (SG1241) was streaked on blood agar and incubated overnight at 37° C. and 5% CO₂.

Expression and Purification of NAPs. The expression and purification of NTHI H-NS, NTHI CbpA, and NTHI HU have been previously described7. Briefly, H-NS, CbpA, and HU were all cloned into pTXB1 vectors and were transformed into the ER2566 E. coli expression strain. Growth took in place in LB with 100 μg/mL ampicillin and then protein overexpression was induced using 100 mM IPTG. Purification of each protein was carried out on a chitin resin column and then further purified using FPLC on a HiTrap Heparin HP column (GE Healthcare).

Quantification of H-NS using Immunofluorescence. Polyclonal anti-H-NS purified from rabbit serum had been produced previously against recombinant NTHI H-NS was used for labelling H-NS in NTHI, S. pneumoniae, and UPEC biofilms of varying ages. All biofilms were started in 8 well glass bottom chamber slides and the media was aspirated and then fresh media was added in 8 hours and 16 hour increments with the exception of week old biofilms where after 5 days the media was replaced every 12 hours. NTHI colonies were resuspended in brain-heart infusion supplemented with 2 μg/mL β-NAD (NAD+) and 2 μg/mL heme (sBHI) broth (BD Diagnostic Systems), UPEC was resuspended in LB, and S. pneumoniae in Todd-Hewitt Broth (THB) (BD Diagnostic Systems) supplemented with 0.2% yeast extract, all resuspensions were added to the chamber slides at a concentration of 2×10⁵ cells/mL. Biofilms were grown for 24 hrs, 40 hrs, 72 hrs, and one week. When the biofilms reached the desired age, they were washed twice with phosphate buffered saline (PBS) and anti-H-NS, rabbit (diluted 1:200 in 5% bovine serum albumin (BSA)) was added for 2 hrs. After incubating at room temperature for 2 hrs, the wells were washed with PBS one time and then goat anti-rabbit Alexa Fluor 488 and FM 4-64 were added (both diluted 1:200 in 5% BSA in PBS) the biofilms were then incubated at room temperature in the dark for 1 hr, the biofilms were then washed with PBS an additional time, and visualized on a Zeiss 800 Light Scanning Microscope (LSM). The mean fluorescence intensity was determined for both H-NS and the cells was determined using ImageJ software.

Visualization of NETs. NETs were isolated from freshly collected blood from healthy donors using EasySep™ Human Neutrophil Isolation Kit from StemCell Technologies (Cambridge, MA). The isolated neutrophils were quantified, and 200,000 cells were added to each well of an 8 well chamber slide. The neutrophils were then allowed to adhere to the bottom of the well for 30 minutes while incubated at 37° C., 5% CO₂. After incubation, 100 nM phorbol 12-myristate 13-acetate (PMA) was added to neutrophils to induce NETosis. For determination if H-NS was able to prevent NETosis, 200 nM NTHI H-NS was added at the same time as PMA and in determining if H-NS could condense previously released NET eDNA, H-NS was added 16 hrs after NETosis was induced. After PMA addition, the neutrophils were incubated for 3.5 hrs at 37° C., 5% CO2. The neutrophils were then fixed with 0.4% formalin and 0.1% Triton X-100 was added for cell permeabilization. The cells were then blocked with 5% normal goat serum for 30 mins at 37° C., 5% CO2 after which primary antibody was added overnight and incubated at 4° C. The primary antibodies utilized α-neutrophil elastase rabbit, α-citrullinated H3 rabbit, and α-DNA mouse, were diluted 1:500 in PBS. The following day the secondary antibody was added for 1 hr at 37° C., 5% CO2. The slides were then visualized on Zeiss 800 Confocal Laser Scanning Microscope.

Quantification of NET Killing. NET Killing was determined by first seeding a 16 hr NTHI biofilm with 2×10⁵ cells/mL. Human neutrophils were then isolated from freshly collected human blood from healthy donors using StemCell EasySep Neutrophil isolation kit. After the 16 hr NTHI biofilm was washed twice with PBS, 2×10⁵ neutrophils were added along with the following proteins: 1 μM H-NS, 1 μM HU, or 1 μM CbpA, no proteins were added to one biofilm and another had no proteins or neutrophils added. The biofilm, proteins (if applicable), and neutrophils (if applicable) were then incubated for 3 hrs at 37° C. After the 3 hr incubation, 0.1% Triton X-100 was added for 5 mins, each sample was then serially diluted and plated on chocolate agar and placed in a 37° C. incubator with 5% CO₂. Colony forming units (CFUs) were then counted after 16 hrs and percentage of bacteria killed was calculated compared to the control without neutrophils added.

RT-PCR to determine levels of H-NS expression. NTHI biofilms were grown for 16 hrs and for one week in a BioLite 25 cm3 tissue flask (ThermoFisher) in 6 mL sBHI media with an initial concentration of 2×10⁵ cells/mL. Before RNA isolation, all surfaces and pipettes were cleaned with RNAaseZap™ (ThermoFisher) The media was removed by turning over the flask and pouring off the media. The biofilm was then resuspended in PBS and centrifuged at 4° C. for 5 minutes at 4000 rpm to pellet bacteria. The bacterial pellet was then resuspended in 0.75 mL Trizol™ (ThermoFisher) for 0.25 mL sample and the homogenized bacteria were allowed to incubate in the Trizol™ for 5 mins at room temperature. After 5 mins, 200 μL of chloroform were added per 1 mL of Trizol™ and the tube containing all reagents and bacteria was shook vigorously for 15 seconds. After shaking, the tube was incubated for 12 minutes at room temperature after which the tube was centrifuged for 5 mins at 4° C. at 4000 rpm. The top clear phase that formed after centrifugation was then removed and put in a new 1.5 mL capped centrifuge tube. RNA purification then was carried out with the Qiagen RNeasy mini kit (Qiagen, Germantown, MD). On-column DNAase I digest (Sigma-Aldrich, St. Louis, MO) was used during the isolation to eliminate residual DNA.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### NON-LIMITING EMBODIMENTS OF THE DISCLOSURE

Embodiment 1. A synthetic polypeptide comprising mB Box-97 that consists of the amino acids 90 to 176 from the coding sequence of the native human HMGB1 protein shown as SEQ ID NO: 2 with a cysteine to serine point mutation at amino acid 106 or an equivalent thereof.

Embodiment 2. A recombinant polypeptide comprising mB Box-97 that consists of the amino acids 90 to 176 from the coding sequence of the native human HMGB1 protein shown as SEQ ID NO: 2 with a cysteine to serine point mutation at amino acid 106 or an equivalent thereof.

Embodiment 3. The synthetic or recombinant polypeptide of embodiment 1 or embodiment 2, wherein the synthetic or recombinant polypeptide consists of SEQ ID NO: 5.

Embodiment 4. A synthetic polypeptide comprising mB Box-97 that consists of the amino acids 80 to 176 from the coding sequence of the native human HMGB1 protein shown as SEQ ID NO: 2 with a cysteine to serine point mutation at amino acid 106 or an equivalent thereof.

Embodiment 5. A recombinant polypeptide comprising mB Box-97 that consists of the amino acids 80 to 176 from the coding sequence of the native human HMGB1 protein shown as SEQ ID NO: 2 with a cysteine to serine point mutation at amino acid 106 or an equivalent thereof.

Embodiment 6. The synthetic or recombinant polypeptide of embodiment 4 or embodiment 5, wherein the synthetic or recombinant polypeptide consists of SEQ ID NO: 6.

Embodiment 7. The synthetic or recombinant polypeptide of any one of embodiments 1-5, wherein an equivalent comprises an amino acid sequence having at least about 80% homology or amino acid identity thereto, or an amino acid encoded by polynucleotide that hybridizes under conditions of high stringency to a polynucleotide encoding the amino acid sequence or its complement, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water; and wherein the equivalent of the cysteine to serine point mutation at amino acid 106 of SEQ ID NO: 2 is a serine to alanine mutation at amino acid 106 of SEQ ID NO: 2.

Embodiment 8. The synthetic or recombinant polypeptide of any of embodiments 1-7, and a detectable label.

Embodiment 9. The synthetic or recombinant polypeptide of any of embodiments 1-8, and a linker polypeptide, optionally wherein the linker polypeptide comprises GPSLKL (SEQ ID NO: 3) or PPKGETKKKF (SEQ ID NO: 4).

Embodiment 10. A plurality of the synthetic or recombinant polypeptide of any of embodiments 1-9, optionally wherein the members of the plurality are the same or different from each other.

Embodiment 11. A composition comprising the synthetic or recombinant polypeptide of any one of embodiments 1-9 or the plurality of embodiment 10, and a carrier, optionally a pharmaceutically acceptable carrier.

Embodiment 12. An isolated polynucleotide encoding the synthetic or recombinant polypeptide of any one of embodiments 1-9, and optionally a carrier or a pharmaceutically acceptable carrier.

Embodiment 13. The isolated polynucleotide of embodiment 12 and a detectable label, and optionally a carrier or a pharmaceutically acceptable carrier.

Embodiment 14. A vector comprising the isolated polynucleotide of embodiment 12 or embodiment 13, and optionally a carrier or a pharmaceutically acceptable carrier.

Embodiment 15. The isolated polynucleotide of embodiment 12 or embodiment 13 or the vector of embodiment 14, further comprising a heterologous promoter sequence, and optionally a carrier or a pharmaceutically acceptable carrier.

Embodiment 16. An isolated host cell containing one or more of: the synthetic or recombinant polypeptide of any one of embodiments 1-9, the plurality of embodiment 10, the isolated polynucleotide of any one of embodiments 12 or 13 or the vector of embodiment 14, and optionally a carrier or a pharmaceutically acceptable carrier.

Embodiment 17. The isolated host cell of embodiment 16, wherein the host cell is a prokaryotic or a eukaryotic cell.

Embodiment 18. The isolated host cell of embodiment 17, wherein the host cell is a eukaryotic cell, optionally wherein the eukaryotic cell is a mammalian cell.

Embodiment 19. A method for treating a subject in need thereof comprising administering to the subject an effective amount of one or more of: (i) the synthetic or recombinant polypeptide of any one of embodiments 1-9, (ii) the plurality of embodiment 10, (iii) the composition of embodiment 11, (iv) the isolated polynucleotide of embodiment 12 or embodiment 13, or (iv) the vector of embodiment 14.

Embodiment 20. A method for treating or preventing aberrant or excessive Neutrophil Extracellular Trap (NET) formation or to prevent a Neutrophil Extracellular Trap (NET)-mediated disease or prevent the progression of NET-mediated disease in a subject in need thereof comprising administering to the subject an effective amount of one or more of: (i) the synthetic or recombinant polypeptide of any one of embodiments 1-9, (ii) the plurality of embodiment 10, (iii) the composition of embodiment 11, (iv) the isolated polynucleotide of embodiment 12 or embodiment 13, or (iv) the vector of embodiment 14.

Embodiment 21. The method of embodiment 19 or 20, wherein the subject is suffering for one or more of a pulmonary disease selected from SARS CoV-2, cystic fibrosis, asthma, chronic obstructive pulmonary disease, tuberculosis, bacterial pneumonia, respiratory syncytial virus bronchiolitis, influenza virus infection, transfusion-related acute lung injury, and mechanical ventilation; sepsis; atherosclerosis; an autoimmune disease selected from systemic lupus erythematosus, rheumatoid arthritis, Type I diabetes mellitus, or small vessel vasculitis; an autoinflammatory disease selected from gout or inflammatory bowel disease, and/or a metabolic disease selected from Type 2 diabetes or obesity, optionally wherein the effective amount is between 50 nM and 2 μM.

Embodiment 22. The method of embodiment 20, wherein the NET-mediated disease comprises a pulmonary disease selected from SARS CoV-2, cystic fibrosis, asthma, chronic obstructive pulmonary disease, tuberculosis, bacterial pneumonia, respiratory syncytial virus bronchiolitis, influenza virus infection, transfusion-related acute lung injury, and mechanical ventilation; sepsis; atherosclerosis; an autoimmune disease selected from systemic lupus erythematosus, rheumatoid arthritis, Type I diabetes mellitus, or small vessel vasculitis; an autoinflammatory disease selected from gout or inflammatory bowel disease, or a metabolic disease selected from Type 2 diabetes or obesity.

Embodiment 23. A method for preventing or treating a bacterial biofilm in a subject in need thereof comprising administering to the subject an effective amount of one or more of: (i) the synthetic or recombinant polypeptide of any one of embodiments 1-9, (ii) the plurality of embodiment 10, (iii) the composition of embodiment 11, (iv) the isolated polynucleotide of embodiment 12 or embodiment 13, or (iv) the vector of embodiment 14, optionally wherein the effective amount is between 50 nM and 2 μM.

Embodiment 24. The method of any one of embodiments 19-23, further comprising administering to the subject an antibody or a fragment thereof, wherein the antibody or fragment thereof binds to a tip region of a DNABII peptide.

Embodiment 25. The method of embodiment 24, wherein the antibody or a fragment thereof comprises:
a heavy chain (HC) immunoglobulin variable domain sequence comprising a sequence of amino acid (aa) 25 to aa 144 of SEQ ID NO: 21 or an equivalent thereof; and
a light chain (LC) immunoglobulin variable domain sequence comprising a sequence of aa 21 to aa 132 of SEQ ID NO: 22 or an equivalent thereof;
or
   wherein the antibody or a fragment thereof comprises:
a heavy chain (HC) immunoglobulin variable domain sequence comprising a sequence of aa 25 to aa 144 of SEQ ID NO: 24 or an equivalent thereof, and
a light chain (LC) immunoglobulin variable domain sequence comprising a sequence of aa 21 to aa 132 of SEQ ID NO: 25 or an equivalent thereof.

Embodiment 26. The method of embodiment 24, wherein the antibody or a fragment thereof comprises:
a heavy chain complementarity-determining region 1 (CDRH1) comprising a sequence of GFTFRTY (SEQ ID NO: 47) (aa 50 to aa 56 of SEQ ID NO: 9 or 10 or 11 or 24);
a heavy chain complementarity-determining region 2 (CDRH2) comprising a sequence of GSDRRH (SEQ ID NO: 48) (aa 76 to aa 81 of SEQ ID NO: 9 or 10 or 11 or 24);
a heavy chain complementarity-determining region 3 (CDRH3) comprising a sequence of VGPYDGYYGEFDY (SEQ ID NO: 49) (aa 121 to aa 133 of SEQ ID NO: 9 or 10 or 11 or 24);
a light chain complementarity-determining region 1 (CDRL1) comprising a sequence of QSLLDSDGKTF (SEQ ID NO: 51) (aa 47 to aa 57 of SEQ ID NO: 15 or 16 or 17 or 25);
a light chain complementarity-determining region 2 (CDRL2) comprising a sequence of LVS (aa 75 to aa 77 of SEQ ID NO: 15 or 16 or 17 or 25); and
a light chain complementarity-determining region 3 (CDRL3) comprising a sequence of WQGTHFP (SEQ ID NO: 52) (aa 114 to aa 120 of SEQ ID NO: 15 or 16 or 17 or 25).

Embodiment 27. A method for condensing eDNA tendrils of a Neutrophil Extracellular Traps (NET) comprising contacting the NET with an effective amount of a DNA-binding agent.

Embodiment 28. A method for preventing or disabling the eDNA structure of a Neutrophil Extracellular Traps (NET) comprising contacting the NET with an effective amount of a DNA-binding agent.

Embodiment 29. A method for preventing a Neutrophil Extracellular Trap (NET) formation or inducing retraction of extant NETs comprising contacting the NET with an effective amount of a DNA-binding agent.

Embodiment 30. The method of any one of embodiments 27-29, wherein the DNA-binding agent is an agent that aggregates or condenses DNA, and optionally wherein the effective amount is between 50 nM and 2 μM.

Embodiment 31. The method of any one of embodiments 27-30, wherein the DNA-binding agent comprises a Histone-like Nucleoid Structuring protein (H-NS), a polyamine, or a polycation.

Embodiment 32. The method of embodiment 31, wherein the H-NS is derived from a gram-negative bacterium or a gram-positive bacterium.

Embodiment 33. The method of embodiment 32, wherein the H-NS is derived from a bacterium of a genus Escherichia, Haemophilus, Streptococcus, Mycobacteria, Klebsiella, or Pseudomonas; optionally wherein the H-NS is derived from E Coli, Nontypeable Haemophilus influenzae (NTHI), S. pneumoniae, K. pneumoniae, Mycobacterium tuberculosis, or Pseudomonas aeruginosa.

Embodiment 34. The method of any one of embodiments 27-33, wherein the H-NS comprises an amino acid sequence having at least 60% identity to an amino acid sequence selected from SEQ ID NOs: 29-34.

Embodiment 35. The method of any one of embodiments 27-34, wherein the contacting is in vitro or in vivo.

Embodiment 36. A method for arresting damaging clotting in a subject in need comprising administering to the subject in need an effective amount of a DNA-binding agent.

Embodiment 37. A method for arresting excessive inflammation in a subject in need comprising administering to the subject in need an effective amount of a DNA-binding agent, optionally wherein the effective amount is between 50 nM and 2 μM.

Embodiment 38. A method for preventing, treating or preventing the progression of a Neutrophil Extracellular Trap (NET)-mediated disease in a subject in need comprising administering to the subject in need an effective amount of a DNA-binding agent, optionally wherein the effective amount is between 50 nM and 2 μM.

Embodiment 39. The method of any one of embodiments 36-38, wherein the DNA-binding agent is an agent that aggregates or condenses DNA.

Embodiment 40. The method of any one of embodiments 36-39, wherein the DNA-binding agent comprises a Histone-like Nucleoid Structuring protein (H-NS).

Embodiment 41. The method of embodiment 40, wherein the H-NS is derived from a gram-negative bacterium or a gram-positive bacterium.

Embodiment 42. The method of embodiment 41, wherein the H-NS is derived from a bacterium of a genus Escherichia, Haemophilus, Streptococcus, Mycobacteria, Klebsiella, or Pseudomonas; optionally wherein the H-NS is derived from E Coli, Nontypeable Haemophilus influenzae (NTHI), S. pneumoniae, K. pneumoniae, Mycobacterium tuberculosis, or Pseudomonas aeruginosa.

Embodiment 43. The method of any one of embodiments 40-42, wherein the H-NS comprises an amino acid sequence having at least 60% identity to an amino acid sequence selected from SEQ ID NOs: 29-34.

Embodiment 44. The method of any one of embodiments 36-43, wherein the subject is a mammal or a human patient.

Embodiment 45. The method of any one of embodiments 36-44, wherein the subject is suffering for one or more of a pulmonary disease selected from SARS CoV-2, cystic fibrosis, asthma, chronic obstructive pulmonary disease, tuberculosis, bacterial pneumonia, respiratory syncytial virus bronchiolitis, influenza virus infection, transfusion-related acute lung injury, and mechanical ventilation; sepsis; atherosclerosis; an autoimmune disease selected from systemic lupus erythematosus, rheumatoid arthritis, Type I diabetes mellitus, or small vessel vasculitis; an autoinflammatory disease selected from gout or inflammatory bowel disease, and/or a metabolic disease selected from Type 2 diabetes or obesity.

1. A synthetic polypeptide comprising mB Box-97 that consists of the amino acids 90 to 176 or amino acids 80 to 176 from the coding sequence of the native human HMGB1 protein shown as SEQ ID NO: 2 with a cysteine to serine point mutation at amino acid 106 or an equivalent thereof, wherein the synthetic polypeptide does not have any post-translational modifications.

2. (canceled)

3. The synthetic polypeptide of claim 1, wherein the synthetic polypeptide consists of SEQ ID NO: 5.

4-5. (canceled)

6. The synthetic polypeptide of claim 1, wherein the synthetic polypeptide consists of SEQ ID NO: 6.

7-10. (canceled)

11. A composition comprising the synthetic polypeptide of claim 1, and a carrier, optionally a pharmaceutically acceptable carrier.

12. An isolated polynucleotide encoding the synthetic polypeptide of claim 1.

13-15. (canceled)

16. An isolated host cell comprising the synthetic polypeptide of claim 1, wherein the host cell is a prokaryotic or a eukaryotic cell.

17-19. (canceled)

20. A method for treating or preventing aberrant or excessive Neutrophil Extracellular Trap (NET) formation or to prevent a Neutrophil Extracellular Trap (NET)-mediated disease or prevent the progression of NET-mediated disease in a subject in need thereof comprising administering to the subject an effective amount of the synthetic polypeptide of claim 1.

21. (canceled)

22. The method of claim 20, wherein the NET-mediated disease comprises a pulmonary disease selected from SARS CoV-2, cystic fibrosis, asthma, chronic obstructive pulmonary disease, tuberculosis, bacterial pneumonia, respiratory syncytial virus bronchiolitis, influenza virus infection, transfusion-related acute lung injury, and mechanical ventilation; sepsis; atherosclerosis; an autoimmune disease selected from systemic lupus erythematosus, rheumatoid arthritis, Type I diabetes mellitus, or small vessel vasculitis; an autoinflammatory disease selected from gout or inflammatory bowel disease, or a metabolic disease selected from Type 2 diabetes or obesity, wherein the effective amount is between 50 nM and 2 μM.

23. (canceled)

24. The method of claim 20, further comprising administering to the subject an antibody or a fragment thereof, wherein the antibody or fragment thereof binds to a tip region of a DNABII peptide, wherein the antibody or a fragment thereof is selected from:
(i) a heavy chain (HC) immunoglobulin variable domain sequence comprising a sequence of amino acid (aa) 25 to aa 144 of SEO ID NO: 21 or an equivalent thereof; and a light chain (LC) immunoglobulin variable domain sequence comprising a sequence of aa 21 to aa 132 of SEO ID NO: 22 or an equivalent thereof;
or
(ii) a heavy chain (HC) immunoglobulin variable domain sequence comprising a sequence of aa 25 to aa 144 of SEO ID NO: 24 or an equivalent thereof; and a light chain (LC) immunoglobulin variable domain sequence comprising a sequence of aa 21 to aa 132 of SEO ID NO: 25 or an equivalent thereof.

25. (canceled)

26. The method of claim 20, further comprising administering to the subject an antibody or a fragment thereof, wherein the antibody or fragment thereof binds to a tip region of a DNABII peptide, and wherein the antibody or a fragment thereof comprises:
(i) a heavy chain complementarity-determining region 1 (CDRH1) comprising a sequence of GFTFRTY (aa 50 to aa 56 of SEQ ID NO: 9 or 10 or 11 or 24);
(ii) a heavy chain complementarity-determining region 2 (CDRH2) comprising a sequence of GSDRRH (aa 76 to aa 81 of SEQ ID NO: 9 or 10 or 11 or 24);
(iii) a heavy chain complementarity-determining region 3 (CDRH3) comprising a sequence of VGPYDGYYGEFDY (aa 121 to aa 133 of SEQ ID NO: 9 or 10 or 11 or 24);
(iv) a light chain complementarity-determining region 1 (CDRL1) comprising a sequence of QSLLDSDGKTF (aa 47 to aa 57 of SEQ ID NO: 15 or 16 or 17 or 25);
(v) a light chain complementarity-determining region 2 (CDRL2) comprising a sequence of LVS (aa 75 to aa 77 of SEQ ID NO: 15 or 16 or 17 or 25); and
(vi) a light chain complementarity-determining region 3 (CDRL3) comprising a sequence of WQGTHFP (aa 114 to aa 120 of SEQ ID NO: 15 or 16 or 17 or 25).

27. A method for (i) condensing eDNA tendrils of a Neutrophil Extracellular Traps (NET), (ii) preventing or disabling the eDNA structure of a Neutrophil Extracellular Traps (NET), or (iii) preventing a Neutrophil Extracellular Trap (NET) formation or inducing retraction of extant NETs comprising contacting the NET with an effective amount of a DNA-binding agent.

28-29. (canceled)

30. The method of claim 27, wherein the DNA-binding agent is an agent that aggregates or condenses DNA, and wherein the effective amount is between 50 nM and 2 μM.

31. The method of claim 27, wherein the DNA-binding agent comprises a Histone-like Nucleoid Structuring protein (H-NS), a polyamine, or a polycation.

32. The method of claim 31, wherein the H-NS is derived from a gram-negative bacterium or a gram-positive bacterium.

33. The method of claim 32, wherein the H-NS is derived from a bacterium of a genus Escherichia, Haemophilus, Streptococcus, Mycobacteria, Klebsiella, or Pseudomonas; optionally wherein the H-NS is derived from E. coli, Nontypeable Haemophilus influenzae (NTHI), S. pneumoniae, K. pneumoniae, Mycobacterium tuberculosis, or Pseudomonas aeruginosa.

34. The method of claim 31, wherein the H-NS comprises an amino acid sequence having at least 60% identity to an amino acid sequence selected from SEQ ID NOs: 29-34.

35. The method of claim 27, wherein the contacting is in vitro or in vivo.

36. A method for (i) arresting damaging clotting, (ii) arresting excessive inflammation, or (iii) preventing, treating or preventing the progression of a Neutrophil Extracellular Trap (NET)-mediated disease in a subject in need comprising administering to the subject in need an effective amount of a DNA-binding agent.

37-38. (canceled)

39. The method of claim 36, wherein the DNA-binding agent is an agent that aggregates or condenses DNA.

40. The method of claim 36, wherein the DNA-binding agent comprises a Histone-like Nucleoid Structuring protein (H-NS) derived from a bacterium of a genus Escherichia, Haemophilus, Streptococcus, Mycobacteria, Klebsiella, or Pseudomonas; optionally wherein the H-NS is derived from E. coli, Nontypeable Haemophilus influenzae (NTHI), S. pneumoniae, K. pneumoniae, Mycobacterium tuberculosis, or Pseudomonas aeruginosa.

41-42. (canceled)

43. The method of claim 40, wherein the H-NS comprises an amino acid sequence having at least 60% identity to an amino acid sequence selected from SEQ ID NOs: 29-34.

44-45. (canceled)

46. A combination or kit comprising the synthetic polypeptide of claim 1 and an antibody or a fragment thereof, wherein the antibody or fragment thereof binds to a tip region of a DNABII peptide, and wherein the antibody or a fragment thereof comprises:
(i) a heavy chain complementarity-determining region 1 (CDRH1) comprising a sequence of GFTFRTY (aa 50 to aa 56 of SEQ ID NO: 9 or 10 or 11 or 24);
(ii) a heavy chain complementarity-determining region 2 (CDRH2) comprising a sequence of GSDRRH (aa 76 to aa 81 of SEQ ID NO: 9 or 10 or 11 or 24);
(iii) a heavy chain complementarity-determining region 3 (CDRH3) comprising a sequence of VGPYDGYYGEFDY (aa 121 to aa 133 of SEQ ID NO: 9 or 10 or 11 or 24);
(iv) a light chain complementarity-determining region 1 (CDRL1) comprising a sequence of QSLLDSDGKTF (aa 47 to aa 57 of SEQ ID NO: 15 or 16 or 17 or 25);
(v) a light chain complementarity-determining region 2 (CDRL2) comprising a sequence of LVS (aa 75 to aa 77 of SEQ ID NO: 15 or 16 or 17 or 25); and
(vi) a light chain complementarity-determining region 3 (CDRL3) comprising a sequence of WQGTHFP (aa 114 to aa 120 of SEQ ID NO: 15 or 16 or 17 or 25).

47. A composition comprising: a carrier, the synthetic polypeptide of claim 1, and an antibody or a fragment thereof, wherein the antibody or fragment thereof binds to a tip region of a DNABII peptide, and wherein the antibody or a fragment thereof comprises:
(i) a heavy chain complementarity-determining region 1 (CDRH1) comprising a sequence of GFTFRTY (aa 50 to aa 56 of SEQ ID NO: 9 or 10 or 11 or 24);
(ii) a heavy chain complementarity-determining region 2 (CDRH2) comprising a sequence of GSDRRH (aa 76 to aa 81 of SEQ ID NO: 9 or 10 or 11 or 24);
(iii) a heavy chain complementarity-determining region 3 (CDRH3) comprising a sequence of VGPYDGYYGEFDY (aa 121 to aa 133 of SEQ ID NO: 9 or 10 or 11 or 24);
(iv) a light chain complementarity-determining region 1 (CDRL1) comprising a sequence of QSLLDSDGKTF (aa 47 to aa 57 of SEQ ID NO: 15 or 16 or 17 or 25);
(v) a light chain complementarity-determining region 2 (CDRL2) comprising a sequence of LVS (aa 75 to aa 77 of SEQ ID NO: 15 or 16 or 17 or 25); and
(vi) a light chain complementarity-determining region 3 (CDRL3) comprising a sequence of WQGTHFP (aa 114 to aa 120 of SEQ ID NO: 15 or 16 or 17 or 25).
